SEQUENCE LISTING (104 sequences)

SEQ ID NO: 1 - protein, 15 aa, synthetic construct
XXXXXEMGFFXXXXX

SEQ ID NO: 2 - protein, 15 aa, synthetic construct
XXXEMGFFXXXXXXX

SEQ ID NO: 3 - protein, 15 aa, synthetic construct
XXXXXXXEMGFFXXX

SEQ ID NO: 4 - protein, 15 aa, synthetic construct
PLPEMGFFTNIPAMV

SEQ ID NO: 5 - protein, 15 aa, synthetic construct
PLYAPEMGFFHVHHL

SEQ ID NO: 6 - protein, 15 aa, synthetic construct
VNYLPEMGFFHTHSL

SEQ ID NO: 7 - protein, 15 aa, synthetic construct
FASEMGFFLVMTMGF

SEQ ID NO: 8 - protein, 15 aa, synthetic construct
MHPSAICEMGFFQDC

SEQ ID NO: 9 - protein, 15 aa, synthetic construct
PGSEMGFFSCRSLPL

SEQ ID NO: 10 - protein, 15 aa, synthetic construct
RLLPPEMGFFKPYWW

SEQ ID NO: 11 - protein, 15 aa, synthetic construct
PGSEMGFFACLWFYS

SEQ ID NO: 12 - protein, 15 aa, synthetic construct
MVPEMGFFEPGLPSP

SEQ ID NO: 13 - protein, 15 aa, synthetic construct
SEPEMGFFVLCAILQ

SEQ ID NO: 14 - protein, 14 aa, synthetic construct
XXXXXXXYPMFXXX

SEQ ID NO: 15 - protein, 14 aa, synthetic construct
SPGTHPKYPMFHLR

SEQ ID NO: 16 - protein, 14 aa, synthetic construct
SQVNPPVYPMFHLH

SEQ ID NO: 17 - protein, 14 aa, synthetic construct
SYSRVPLYPMFVNR

SEQ ID NO: 18 - protein, 14 aa, synthetic construct
SYRSDLVYPMFIRW

SEQ ID NO: 19 - protein, 14 aa, synthetic construct
SHTRHPVYPMFHPR

SEQ ID NO: 20 - protein, 14 aa, synthetic construct
SQSPTIFYPMFSLR

SEQ ID NO: 21 - protein, 14 aa, synthetic construct
SQSERPVYPMFHLR

SEQ ID NO: 22 - protein, 14 aa, synthetic construct
SRSERPVYPMFHLR

SEQ ID NO: 23 - protein, 140 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRSHTRHPVYPMFHPRDYRGQGTLVTVSSGAAEQKLIFEEDL

SEQ ID NO: 24 - protein, 18 aa, synthetic construct
XXXXXWXXSRTPWXXXXX

SEQ ID NO: 25 - protein, 18 aa, synthetic construct
XXXXXXXWXXSRTPWXXX

SEQ ID NO: 26 - protein, 18 aa, synthetic construct
XXXWXXSRTPWXXXXXXX

SEQ ID NO: 27 - protein, 18 aa, synthetic construct
LTWTRWVRSRTPWNVMGF

SEQ ID NO: 28 - protein, 18 aa, synthetic construct
LVQRLTRWRFSRTPWHFW

SEQ ID NO: 29 - protein, 18 aa, synthetic construct
AHYWRFSRTPWFVRLTHQ

SEQ ID NO: 30 - protein, 18 aa, synthetic construct
IQRWRLSRTPWSRSFHRI

SEQ ID NO: 31 - protein, 18 aa, synthetic construct
SLRWINSRTPWWIVRRML

SEQ ID NO: 32 - protein, 18 aa, synthetic construct
TERWHDSRTPWANLYRHF

SEQ ID NO: 33 - protein, 18 aa, synthetic construct
SVKWLNSRTPWTRMMYTL

SEQ ID NO: 34 - protein, 18 aa, synthetic construct
RSAGLWRYSRTPWYLLRT

SEQ ID NO: 35 - protein, 18 aa, synthetic construct
QLRWSTSRTPWAKLAVKP

SEQ ID NO: 36 - protein, 18 aa, synthetic construct
VSSVRWFRSRTPWKMLHH

SEQ ID NO: 37 - protein, 18 aa, synthetic construct
NFRWVWSRTPWHMRADVR

SEQ ID NO: 38 - protein, 18 aa, synthetic construct
CYKWASSRTPWARLWATT

SEQ ID NO: 39 - protein, 18 aa, synthetic construct
ERKWRFSRTPWRWSHSRY

SEQ ID NO: 40 - protein, 18 aa, synthetic construct
SHHWFGSRTPWSWSNTLR

SEQ ID NO: 41 - protein, 18 aa, synthetic construct
STKWSESRTPWALLRLSP

SEQ ID NO: 42 - protein, 18 aa, synthetic construct
YPWLRWRLSRTPWSLRWS

SEQ ID NO: 43 - protein, 18 aa, synthetic construct
GRAYPWRWSRTPWYRTRL

SEQ ID NO: 44 - protein, 18 aa, synthetic construct
NLRAPYRWLKSRTPWANM

SEQ ID NO: 45 - protein, 18 aa, synthetic construct
AIGRWWGWSRTPWRFPLP

SEQ ID NO: 46 - protein, 18 aa, synthetic construct
SSRWTNSRTPWTLISLSD

SEQ ID NO: 47 - protein, 18 aa, synthetic construct
VISWRWSRTPWSLRAKNR

SEQ ID NO: 48 - protein, 18 aa, synthetic construct
VISWRWSRTPWSLRAKNR

SEQ ID NO: 49 - protein, 18 aa, synthetic construct
AARWTNSRTPWSTYRWLK

SEQ ID NO: 50 - protein, 18 aa, synthetic construct
KYVWKNSRTPWRRLTHNT

SEQ ID NO: 51 - protein, 18 aa, synthetic construct
TATERWRNSRTPWTLIKV

SEQ ID NO: 52 - protein, 18 aa, synthetic construct
QLRWLKSRTPWARVMLVL

SEQ ID NO: 53 - protein, 18 aa, synthetic construct
VARRRWLGSRTPWVGKYS

SEQ ID NO: 54 - protein, 18 aa, synthetic construct
GLRWRLSRTPWAAHLARG

SEQ ID NO: 55 - protein, 18 aa, synthetic construct
SLRWRASRTPWNKLFVIL

SEQ ID NO: 56 - protein, 18 aa, synthetic construct
FLRWTLSRTPWTVRAKNP

SEQ ID NO: 57 - protein, 18 aa, synthetic construct
QWRWTWSRTPWRRYRRLS

SEQ ID NO: 58 - protein, 18 aa, synthetic construct
MWRWVFSRTPWRRIQRDT

SEQ ID NO: 59 - protein, 18 aa, synthetic construct
ITRWVNSRTPWHALLLLA

SEQ ID NO: 60 - protein, 16 aa, synthetic construct
NFRWLNSRTPWTRANP

SEQ ID NO: 61 - protein, 18 aa, synthetic construct
GCLWVLSRTPWCMLKLGP

SEQ ID NO: 62 - protein, 14 aa, synthetic construct
YVRPRLPHMSQFES

SEQ ID NO: 63 - protein, 15 aa, synthetic construct
ETCPYVESRTPWCRQ

SEQ ID NO: 64 - protein, 9 aa, synthetic construct
CETGFFTGC

SEQ ID NO: 65 - protein, 9 aa, synthetic construct
CEMGFFQDC

SEQ ID NO: 66 - protein, 9 aa, synthetic construct
CELGYYNDC

SEQ ID NO: 67 - protein, 9 aa, synthetic construct
CETGFFLKC

SEQ ID NO: 68 - protein, 9 aa, synthetic construct
CELGFYRLC

SEQ ID NO: 69 - protein, 9 aa, synthetic construct
CETGFFLRC

SEQ ID NO: 70 - protein, 9 aa, synthetic construct
CETGYFSQC

SEQ ID NO: 71 - protein, 9 aa, synthetic construct
CIHSPTSLC

SEQ ID NO: 72 - protein, 9 aa, synthetic construct
CETGFYKTC

SEQ ID NO: 73 - protein, 9 aa, synthetic construct
CEMGYFGNC

SEQ ID NO: 74 - protein, 9 aa, synthetic construct
CAMGFFQDC

SEQ ID NO: 75 - protein, 9 aa, synthetic construct
CEAGFFQDC

SEQ ID NO: 76 - protein, 9 aa, synthetic construct
CEMAFFQDC

SEQ ID NO: 77 - protein, 9 aa, synthetic construct
CEMGAFQDC

SEQ ID NO: 78 - protein, 9 aa, synthetic construct
CEMGFAQDC

SEQ ID NO: 79 - protein, 9 aa, synthetic construct
CEMGFFADC

SEQ ID NO: 80 - protein, 9 aa, synthetic construct
CEMGFFQAC

SEQ ID NO: 81 - protein, 10 aa, synthetic construct
CEMGFFQDCG

SEQ ID NO: 82 - protein, 10 aa, synthetic construct
ACEMGFFQDC

SEQ ID NO: 83 - protein, 9 aa, synthetic construct
CEMGFFQDC

SEQ ID NO: 84 - protein, 15 aa, synthetic construct
PLPEAGFFTNIPAMV

SEQ ID NO: 85 - protein, 12 aa, synthetic construct
SLTYTREFLRSL

SEQ ID NO: 86 - protein, 12 aa, synthetic construct
AQSYTRMELLRL

SEQ ID NO: 87 - protein, 12 aa, synthetic construct
HYSRTDLLAYRW

SEQ ID NO: 88 - protein, 12 aa, synthetic construct
SGIQQRMIVTWP

SEQ ID NO: 89 - protein, 12 aa, synthetic construct
KLWSIPTNFLLP

SEQ ID NO: 90 - protein, 12 aa, synthetic construct
VEGQNLEALVTD

SEQ ID NO: 91 - protein, 12 aa, synthetic construct
TISYDRSTLFLF

SEQ ID NO: 92 - protein, 12 aa, synthetic construct
DSAKERLAVSGT

SEQ ID NO: 93 - protein, 12 aa, synthetic construct
LPRMMSDGEAGD

SEQ ID NO: 94 - protein, 12 aa, synthetic construct
YLMQTNQWQIAS

SEQ ID NO: 95 - protein, 13 aa, synthetic construct
GGSEMGFFGGSGG

SEQ ID NO: 96 - protein, 13 aa, synthetic construct
GGSGGEMGFFGGS

SEQ ID NO: 97 - protein, 13 aa, synthetic construct
GGSGEMGFFGGSG

SEQ ID NO: 98 - protein, 131 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRPLPEMGFFTNIPAMVDYRGQGTLVTVSSGAA

SEQ ID NO: 99 - protein, 139 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRVAKADLNSSSPSFVVNTYSSFGFDYRGQGTLVTVSSGAA

SEQ ID NO: 100 - protein, 138 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRVAKALNSSSPSFVVNTYSSFGFDYRGQGTLVTVSSGAA

SEQ ID NO: 101 - protein, 138 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRVAKALNSRSPSFVVNTYSSIGFDYRGQGTLVTVSSGAA

SEQ ID NO: 102 - protein, 131 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRPLPEAGFFTNIPAMVDYRGQGTLVTVSSGAA

SEQ ID NO: 103 - protein, 131 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRPLPAMGFFTNIPAMVDYRGQGTLVTVSSGAA

SEQ ID NO: 104 - protein, 138 aa, synthetic construct
MSEVQLVESGGGLVQPGGSLRLSSAISGFSISSTSIDWVRQAPGKGLEWVARISPSSGSTSYADSVKGRFTISADTSKNTVYLQMNSLRAEDTAVYYTGRGSVLSVAFKPSVDSFSSFYNTNDYRGQGTLVTVSSGAA

(19) United States
(12) Patent Application Publication
(10) Pub. No.: US 2025/0346630 A1
(43) Pub. Date: Nov. 13, 2025

(54) Engineering Peptides Using Peptide Epitope Linker Evolution

(71) Applicant: AGENCY FOR SCIENCE, TECHNOLOGY AND RESEARCH, Singapore (SG)

(72) Inventors: Christopher John Brown, Singapore (SG); Yuri Frosi, Singapore (SG); Simon Ng, Singapore (SG); Yen-Chu Lin, Singapore (SG)

(21) Appl. No.: 18/850,447

(22) PCT Filed: Mar. 24, 2023
(86) PCT No.: PCT/SG2023/050200

§ 371 Date: Sep. 24, 2024

(30) Foreign Application Priority Data
Mar. 24, 2022 (SG) 10202203066U

(51) Int. Cl. C07K 7/08; A61K 38/10; A61P 35/00

(52) U.S. Cl. CPC C07K 7/08; A61K 38/10; A61P 35/00; C07K 2317/56

(57) ABSTRACT

The present invention relates to methods of engineering and identifying a peptide aptamer that binds to a target protein of interest, and peptide aptamers engineered and identified using these methods and methods to identify a candidate peptide or nucleic acid that binds to a target protein in a live cell. The peptide aptamers defined herein may be useful for treating a condition associated with dysregulated cap-dependent translation, dysregulated DNA replication, dysregulated DNA repair and/or dysregulated mRNA translation such as cancer, diseases associated with a viral infection and obesity.

### CROSS-REFERENCE TO RELATED APPLICATIONS

This application claims the benefit of priority of Singapore Application No. 10202203066U, filed 24 Mar. 2022, the contents of it being hereby incorporated by reference in its entirety for all purposes.

### FIELD OF THE INVENTION

The present invention relates to peptide aptamers and their uses thereof. The present invention also relates to methods of engineering and identifying peptide aptamers that display high specificity to target proteins. The present invention further discloses methods of developing protein-protein interaction assays for screening antagonists in live cells.

### SEQUENCE LISTING

The instant application contains a Sequence Listing which has been submitted electronically in XML format and is hereby incorporated by reference in its entirety. Said XML copy, created on 6 Feb. 2023, is named 77743PCT_Sequence Listing.xml and is 90.2 kilobytes in size.

### BACKGROUND OF THE INVENTION

Peptides are ideal modalities for identifying novel binding sites due to their ability to adopt multiple configurations, mimic molecular features at protein binding interfaces, and interact with their target molecules with relatively high affinities and specificities. In addition, the range of these binding sites could be extended by constraining the secondary protein structure through cyclization. These desirable biological properties of the peptides make them attractive as potential therapeutic molecules.

However, the translation of the peptides into therapeutic molecules poses difficulties which are limited cell permeability and proteolytic instability. To circumvent these difficulties, peptide aptamers are engineered whereby the binding epitopes are effectively presented within the context of a scaffold protein and these epitopes are doubly constrained at both the N and C terminals. Strategies for designing peptide aptamers include the insertion of a single amino acid sequence into a hypervariable loop or mutation of specific residues embedded in the rigid secondary structural elements within the scaffold protein.

Although the existing methodological approach and the rational protein engineering techniques to engineer and identify novel peptide aptamers which are capable of modulating the activity of the target protein are amenable to combinatorial display methods including yeast and phage display, there are limitations which include 1) specific or hypervariable loop sequences being inserted into scaffolds that are not optimized to stabilize their conformations when bound to the target molecule and 2) the use of “rigid motifs” leading to sampling limited areas of 3-dimensional space e.g., mutations that lie down one side of an α-helix, thus affecting the binding affinity to the target molecule.

Therefore, there is a need to develop methods of engineering and identifying peptide aptamers that display improved cell permeability and stability whilst retaining high binding affinity to the target protein.

### SUMMARY

In one aspect, there is provided a method of identifying and isolating a peptide aptamer (PA) that is capable of binding to a target protein comprising:
a) identifying a peptide motif that interacts with the target protein from a library of peptides;
b) inserting a hypervariable region in a scaffold protein;
c) inserting the peptide motif into a plurality of positions in the hypervariable region of the scaffold protein to generate one or more library of peptide aptamers, wherein the hypervariable region forms one or more linkers between the peptide motif and the scaffold protein;
d) contacting the library from step c with the target protein to identify the peptide aptamer that is capable of binding to the target protein;
e) isolating the peptide aptamer from step d.

In another aspect, there is provided a peptide aptamer comprising an amino acid sequence selected from the group consisting of:

i)
(SEQ ID NO: 1)
X₁X₂X₃X₄X₅EMGFFX₆X₇X₈X₉X₁₀
ii)
(SEQ ID NO: 2)
X₁₁X₁₂X₁₃EMGFFX₁₄X₁₅X₁₆X₁₇X₁₈X₁₉X₂₀,
and
ii)
(SEQ ID NO: 3)
X₂₁X₂₂X₂₃X₂₄X₂₅X₂₆X₂₇EMGFFX₂₈X₂₉X₃₀;

wherein X₁₋₃₀ is any amino acid;
wherein X₁₋₅, X₁₁₋₁₃ and X₂₁₋₂₇ comprise amino acid sequences for a first linker;
wherein X₆₋₁₀, X₁₄₋₂₀ and X₂₈₋₃₀ comprise amino acid sequences for a second linker;
optionally wherein X₅, X₁₃ and X₂₇ is an amino acid selected from the group consisting of proline (P), serine (S) and cysteine (C);
optionally wherein X₆, X₁₄ and X₂₈ is an amino acid selected from the group consisting of histidine (H), threonine (T), leucine (L), glutamine (Q), serine (S), lysine (K), alanine (A), glutamate (E) and valine (V); and
optionally where in X₇, X₁₅ and X₂₉ is an amino acid selected from the group consisting of asparagine (N), valine (V), threonine (T), aspartate (D), cysteine (C), proline (P) and leucine (L);
wherein the peptide aptamer binds to eIF4E.

In another aspect, there is provided a peptide aptamer comprising an amino acid sequence X₁X₂X₃X₄X₅X₆X₇YPMFX₈X₉X₁₀ (SEQ ID NO:14);
wherein X₁-X₁₀ is any amino acid;
wherein X₁₋₇ comprises amino acid sequences for a first linker;
wherein X₈₋₁₀ comprises amino acid sequences for a second linker;
optionally wherein X₁ is serine (S);
optionally wherein X₂ is an amino acid selected from the group consisting of proline (P), glutamine (Q), tyrosine (Y), histidine (H) and arginine (R);
optionally wherein X₃ is an amino acid selected from the group consisting of glycine (G), valine (V), serine (S), arginine (R) and threonine (T);
optionally wherein X₄ is an amino acid selected from the group consisting of threonine (T), asparagine (N), arginine (R), serine (S), proline (P) and glutamate (E);
optionally wherein X₅ is an amino acid selected from the group consisting of histidine (H), proline (P), valine (V), aspartate (D), threonine (T) and arginine (R);
optionally wherein X₆ is an amino acid selected from the group consisting of proline (P), leucine (L) and isoleucine (I);
optionally wherein X₇ is an amino acid selected from the group consisting of lysine (K), valine (V), leucine (L) and phenylalanine (F);
optionally wherein X₈ is an amino acid selected from the group consisting of histidine (H), valine (V), isoleucine (I) and serine (S);
optionally wherein X₉ is an amino acid selected from the group consisting of leucine (L), asparagine (N), arginine (R) and proline (P); and
optionally wherein X₁₀ is an amino acid selected from the group consisting of arginine (R), histidine (H) and tryptophan (W);
wherein the peptide aptamer binds to proliferating cell nuclear antigen (PCNA).

In another aspect, there is provided a peptide aptamer comprising an amino acid sequence selected from the group consisting of

i)
(SEQ ID NO: 24)
X₁X₂X₃X₄X₅WX₆X₇SRTPWX₈X₉X₁₀X₁₁X₁₂,
ii)
(SEQ ID NO: 25)
X₁₃X₁₄X₁₅X₁₆X₁₇X₁₈X₁₉WX₂₀X₂₁SRTPWX₂₂X₂₃X₂₄,
and
iii)
(SEQ ID NO: 26)
X₂₅X₂₆X₂₇WX₂₈X₂₉SRTPWX₃₀X₃₁X₃₂X₃₃X₃₄X₃₅X₃₆;

wherein X₁-X₃₆ is any amino acid;
wherein X₁₋₅, X₁₃₋₁₉ and X₂₅₋₂₉ comprise amino acid sequences for a first linker;
wherein X₈₋₁₂, X₂₂₋₂₄ and X₃₀₋₃₆ comprise amino acid sequences for a second linker;
optionally wherein X₁ and X₁₅ is an amino acid selected from the group consisting of leucine (L), glutamine (Q), arginine (R), valine (V), tyrosine (Y), glycine (G), alanine (A) and threonine (T);
optionally wherein X₂ and X₁₆ is an amino acid selected from the group consisting of threonine (T), arginine (R), serine (S), proline (P), alanine (A) and isoleucine (I);
optionally wherein X₃, X₁₇ and X₂₅ is an amino acid selected from the group consisting of tryptophan (W), leucine (L), alanine (A), isoleucine (I), serine (S), threonine (T), glutamine (Q), asparagine (N), glutamate (E), cysteine (C), proline (P), glycine (G), valine (V), arginine (K), arginine (R), phenylalanine (F), and methionine (M);
optionally wherein X₄, X₁₈ and X₂₆ is an amino acid selected from the group consisting of threonine (T), histidine (H), glutamine (Q), leucine (L), glutamate (E), valine (V), glycine (G), arginine (R), phenylalanine (F), tyrosine (Y), serine (S), isoleucine (I), alanine (A), tryptophan (W) and cysteine (C);
optionally wherein X₅, X₁₉ and X₂₇ is an amino acid selected from the group consisting of arginine (R), tyrosine (Y), lysine (K), leucine (L), histidine (H), proline (P), serine (S), tryptophan (W) and valine (V);
optionally wherein X₆, X₂₀ and X₂₈ is an amino acid selected from the group consisting of valine (V), arginine (R), isoleucine (I), histidine (H), leucine (L), serine (S), phenylalanine (F), alanine (A), glycine (G), threonine (T) and lysine (K);
optionally wherein X₇, X₂₁ and X₂₉ is an amino acid selected from the group consisting of asparagine (N), phenylalanine (F), tryptophan (W), leucine (L), alanine (A), glycine (G), lysine (K), glutamate (E), serine (S), arginine (R), threonine (T), tyrosine (Y) and aspartate (D);
optionally wherein X₈, X₂₂ and X₃₀ is an amino acid selected from the group consisting of asparagine (N), histidine (H), phenylalanine (F), serine (S), tryptophan (W), alanine (A), threonine (T), tyrosine (Y), lysine (K), arginine (R), valine (V) and cysteine (C);
optionally wherein X₉, X₂₃ and X₃₁ is an amino acid selected from the group consisting of valine (V), phenylalanine (F), arginine (R), isoleucine (I), asparagine (N), leucine (L), lysine (K), methionine (M), tryptophan (W), threonine (T), glycine (G) and alanine (A);
optionally wherein X₁₀, X₂₄ and X₃₂ is an amino acid selected from the group consisting of isoleucine (I), arginine (R), tyrosine (Y), leucine (L), valine (V), lysine (K), histidine (H), alanine (A), methionine (M), tryptophan (W), serine (S), threonine (T) and proline (P);
optionally wherein X₁₁ and X₃₃ is an amino acid selected from the group consisting of glycine (G), leucine (L), phenylalanine (F), arginine (R), tyrosine (Y), methionine (M), alanine (A), histidine (H), tryptophan (W), asparagine (N), lysine (K), serine (S), threonine (T) and glutamine (Q);
optionally wherein X₁₂ and X₃₄ is an amino acid selected from the group consisting of phenylalanine (F), threonine (T), histidine (H), arginine (R), tyrosine (Y), valine (V), aspartate (D), alanine (A), serine (S), leucine (L), proline (P), lysine (K) and tryptophan (W);
optionally wherein X₁₃ is an amino acid selected from the group consisting of asparagine (N) and leucine (L);
optionally wherein X₁₄ is an amino acid selected from the group consisting of valine (V), and leucine (L);
optionally wherein X₃₅ is an amino acid selected from the group consisting of (H), arginine (R), methionine (M), threonine (T), lysine (K), valine (V), leucine (L), serine (S), asparagine (N), isoleucine (I) aspartate (D) and glycine (G); and
optionally wherein X₃₆ is an amino acid selected from the group consisting of glutamine (Q), isoleucine (I), leucine (L), phenylalanine (F), proline (P), arginine (R), threonine (T), tyrosine (Y), aspartate (D), lysine (K), glycine (G), serine (S) and alanine (A);
wherein the peptide aptamer binds to eIF4A.

In yet another aspect, there is provided the peptide aptamer as described herein for use as a medicament.

In yet another aspect, there is provided a use of the peptide aptamer as described herein in the manufacture of a medicament for treating a condition associated with dysregulated cap-dependent translation, dysregulated DNA replication, dysregulated DNA repair and/or dysregulated mRNA translation.

In yet another aspect, there is provided a method of treating a condition associated with dysregulated cap-dependent translation, dysregulated DNA replication, dysregulated DNA repair and/or dysregulated mRNA translation, comprising administering the peptide aptamer as described herein to a subject in need thereof, optionally wherein the peptide aptamer is administered as a combinatorial treatment with immunotherapy.

In yet another aspect, there is provided a method to identify a candidate peptide or nucleic acid that binds to a target protein in a live cell comprising:
a) fusing LargeBiT (LgBiT) to the target protein and fusing Small BiT (SmBiT) to a peptide aptamer that binds to the target protein, or fusing SmBiT to the target protein and fusing LgBiT to a peptide aptamer that binds to the target protein;
b) (i) expressing the fusion proteins from step a in the live cell; measuring the level of signal emitted in step b(i), wherein a signal is emitted when the target protein binds to the peptide aptamer;
   contacting the cell expressing the bound fusion protein from step b(i) with the candidate peptide or nucleic acid, optionally wherein the cell is permeabilized prior to contacting with the candidate peptide or nucleic acid; or
   (ii) expressing the fusion proteins from step a and the candidate peptide or nucleic acid in the live cell;
   measuring the level of signal emitted in step b(ii), wherein a signal is emitted when the target protein binds to the peptide aptamer; and
c) detecting a change in level of signal relative to a reference, wherein a decrease in the level of signal emitted is indicative of binding of the candidate peptide or nucleic acid to the target protein.

### Definitions

As used herein, the term “peptide aptamer” refers to an artificial molecule in which a peptide sequence or motif, with affinity for a given target protein, is displayed on a supporting scaffold protein. A peptide aptamer comprises a scaffold protein, a peptide sequence or motif, and may optionally comprise additional linker sequences. Peptide aptamers vary in length and may range from about 10-250 amino acid residues. The terms “peptide aptamer” and “mini protein” can be used interchangeably in this context.

As used herein, the term “scaffold protein” refers to a protein comprising 1) a small single-chain protein that facilitates the application of most selection technologies and the subsequent construction of fusion proteins (e.g. the incorporation of elements such as localization signals, luciferases and epitope and purification tags); 2) rigid, compact, preferably monomeric, stable protein core that is capable of displaying variable target interaction surfaces in a manner analogous to the immunoglobulin complementarity determining region; 3) high thermodynamic stability and the absence of disulphide bonds or free cysteines which are advantageous for the expression of functional molecules in the reducing environment of the bacterial or mammalian cytoplasm; and 4) permutations introduced into variable regions do not adversely affect solubility, folding and the aggregating properties of the resulting combinatorial product.

As used herein is the term “processing” in the context of a peptide aptamer refers to the process of maturation and chemical modification of a peptide aptamer. Maturation of a peptide aptamer comprises post-translational modifications of the peptide aptamer. Post-translational modifications include phosphorylation, acetylation, hydroxylation and methylation. Chemical modification refers to the modification, addition and removal of macromolecules through a chemical reaction. In this context, macromolecules may be peptides, nucleic acids and carbohydrates.

The term “dissociation constant” (K_{d}) as used herein is a measure of the strength of binding between two molecules, for example, a protein and its ligand. The smaller the dissociation constant, the more tightly bound the two molecules are and the higher the affinity between the two molecules.

The term “complex” refers to an association between two or more interacting constituents which may be transient or permanent. A constituent may interact with one or more other constituents of a complex. A complex may be a macromolecular complex. The macromolecular complex includes but not limited to a peptide, a polypeptide, an oligonucleotide and a nucleic acid. In this context, the complex is a peptide. The interactions between the constituents of a complex may be non-covalent or covalent. These non-covalent interactions include but are not limited to van der Waals interactions, electrostatic (ionic) interactions, hydrogen bonds and/or hydrophobic packing. The constituents of a complex may be linked by covalent bonds such as disulphide bonds and amide bonds.

As used herein, the term “inhibit” means that the level of activity is disrupted, reduced or absent compared to the level of activity of the reference that is not inhibited.

As used herein, the term “dysregulated” refers to the alteration, impairment and disruption of normal physiological function. For instance, in the context of cap-dependent translation, dysregulated DNA replication, dysregulated DNA repair and/or dysregulated mRNA translation, the level of activity may be increased or decreased compared to the level of baseline activity.

The term “cancer” as used herein refers to any of a number of diseases characterized by uncontrollable and abnormal proliferation of cells, the ability of affected cells to spread locally or invade other parts of the body through the bloodstream and the lymphatic system (i.e. metastasis). Examples of cancers include but are not limited to melanoma, triple negative breast cancer, lung cancer, colorectal cancer or prostate cancer.

The term “viral infection” refers to the invasion in tissues of the host by a virus. Virus refers broadly to an infectious agent that replicates within the cells of other organisms. Viruses may be classified based on their nucleic acid (RNA or DNA), regardless whether the nucleic acid is single stranded or double stranded, whether reverse transcriptase is utilized, and if their nucleic acid is single stranded RNA, whether it is sense (+) or antisense (−). Viruses can be classified by family, genus, species, and serotype. In this context, the virus is an RNA virus. Examples of an RNA virus include but are not limited to coronavirus, orthomyxovirus, rhabdovirus, reovirus, hantavirus and alphavirus.

The term “administering” and variations of that term including “administer” and “administration”, includes contacting, applying, delivering or providing a composition of the disclosure to subject by any appropriate means.

The term “subject” refers to a human or non-human mammal. Examples of such mammals include but are not limited to a primate, a mouse, a rat, a guinea pig, a rabbit, and a dog. In a preferred example, the subject is a human. The subject may be at risk of virus infection or desired to be treated using the immunogenic compositions and methods described herein.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value. Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### BRIEF DESCRIPTION OF THE DRAWINGS

The invention will be better understood with reference to the detailed description when considered in conjunction with the non-limiting examples and the accompanying drawings, in which:

FIG. 1. shows a schematic diagram of the process of Peptide Epitope Linker Evolution (PELE).

FIG. 2. shows the process to identify and isolate peptide aptamers that display binding affinity to the target protein of interest. FIG. 2A shows the process termed Peptide Epitope Linker Evolution (PELE). Peptide phage display libraries are used to probe the surface of the target protein to discover new binding motifs and modalities (e.g., linear or constrained libraries). Upon identification, the novel motif or modality can be inserted into a larger hypervariable loop located on a selected scaffold protein Several distinctive libraries can then be constructed with the interaction motif (‘peptide epitope’) located at different positions within the hypervariable loop, and the selection against the target protein can be re-performed to select for sequences (‘linker evolution’) that optimally present the interaction motif within the context of the scaffold. FIG. 2B shows a brief outline of next generation sequencing (NGS) enhanced phage display. A selected phage library is panned against an immobilised target protein using three technical repeats and in parallel against corresponding negative control selections where the target protein is either removed or replaced with a different target protein. Bound phage is then eluted, amplified and sequenced using NGS protocols (NextSEQ, Illumina). The FASTQ file generated from the sequencing data was processed by in-house PYTHON scripts that identified the barcodes and constant flanking regions and extracted the reads of the correct length corresponding to the variable peptide library. The table presents the list of sequences identified from each selection with their associated abundance. The abundance is calculated by taking the copy number of each sequence and normalizing it by dividing the copy number by the total number of reads in each sequence. Sequences not observed in a specific replicate were assigned a copy number of zero. The enrichment ratio of each sequence in the target selection was calculated by determining the mean fraction from the target screen replicates and dividing it by the mean fraction from the selected control screen replicates. Since the denominator must not be a zero when taking the ratio, sequences with zero copy number found in all three replicates are assigned with an arbitrary copy number before taking the normalization. Significance of the ratio was assessed using one-tailed, unequal variance Welch test. A heat map (FIGS. 2C and 2D) is then generated to identify the enriched peptides that have ratio and p-values corresponding to the parameters stated in the figure. Each individual block on the map represents the abundance of the unique sequence in each selection and the sequence are ordered by their ratio value. FIG. 2C shows the heatmap showing sequences enriched from the M13 disulphide constrained 7mer library (C7C) against eIF4E, but not in the 2 control selections (Mdm2 and eIF4A). FIG. 2D shows the heatmap showing sequences enriched from the M13 linear 12mer library against eIF4E, but not in the 3 control selections (Mdm2, K-RAS and DO-1). The sequence motif in FIG. 2C was generated from the enriched sequences using MEME, whilst in FIG. 2D it was generated from the sequences exhibiting the known eIF4E binding motif (YXXXL, X is any amino acid).

FIG. 3 shows the binding affinity of the identified cyclic peptides to the target protein. FIG. 3A shows a surface representation of eIF4E depicting the location of the m⁷GTP (capped mRNA) and eIF4G1/4E-BP1 binding sites. Locations of tryptophan residues whose intrinsic fluorescence is sensitive to binding by either m⁷GTP or peptides that interact with the eIF4G binding site are shown in green. FIG. 3B shows competitive fluorescence anisotropy experiments with FAM labelled m⁷GTP assessing binding of the cyclic peptides to the cap-binding site. FIG. 3C shows competitive fluorescence anisotropy experiments with FAM labelled eIF4G1 derived peptide assessing binding of the cyclic peptides to the eIF4G/4E-BP1 binding site. Apparent K_{ds} (see Table 2) were determined by curve-fitting using Prism (Graphpad, Ltd). See materials and methods. FIG. 3D shows that eIF4E intrinsic tryptophan fluorescence was assessed in response to titrations of m⁷GTP, PHAGESOL (Ac-KKRYSR*QLL*-NH₂) and EE-02 (SEQ ID NO: 65), respectively. For PHAGESOL (Ac-KKRYSR*QLL*-NH₂), * represents c-alpha methyl phenylalanine.

FIG. 4 shows the interaction of the peptide motif and the binding site at the target protein. FIG. 4A shows the 2Fₒ-F_{c} electron density map (1.2σ) showing the EE-02 disulfide constrained peptide bound to eIF4E at the cap-binding site. FIG. 4B shows the complex of eIF4E bound to m⁷GTP (PDB ID: 2V8W) indicating conformational differences with the EE-02:eIF4E complex structure. FIG. 4C shows EE-02 when bound to eIF4E forms a β-hairpin turn-like structure that is stabilized by intra hydrogen bonds between the backbone carbonyl of E3 and backbone amide of F6, and the backbone amide of E-03 and carbonyl of F6 (3.1 Å and 4.1 Å, respectively). The conformation of the cyclic peptide is further rigidified by hydrogen bonds between the C10 amide and the carbonyl of F7, and the backbone of N9 and the carbonyl of F7 (3.0 Å and 3.8 Å, respectively). The polypeptide backbone of EE-02 also forms a set of critical interactions with eIF4E (<3.2 Å) shown in dashed lines. FIG. 4D to 4G show the interactions made by the conserved residues of the cyclic peptide interaction motif in EE-02 with eIF4E. FIG. 4D shows E3 electrostatically interacts with R112 and forms a water mediated hydrogen bond interaction with N155. FIG. 4F shows that the carbonyl group of G5 forms no direct interactions with eIF4E but forms a hydrogen bond with a structured water, which is part of a larger network of structured waters that facilitates the interaction of EE-02 with eIF4E. FIG. 4F shows that M4 forms a dipole interaction with the hydroxyl group of S92 and a variety of hydrophobic contacts with residues F48, W46, L60 and P100. FIG. 4G shows that F6 forms hydrophobic contacts with the residues T203, A204, H200, W166 and W102 of eIF4E. FIG. 4H shows the stacking interactions with W56 and edge on face interactions with F48. Additionally, it forms a hydrophobic contact with P100. FIG. 4I shows the overlay of the EE-02:eIF4E complex with unbound eIF4E (PDB ID: 4BEA) and m⁷GTP bound eIF4E demonstrating the similarity of the EE-02 bound conformation to the apo structure. Ligands interacting at the cap binding site (EE-02 and m⁷GTP are not shown for clarity).

FIG. 5 shows the libraries generated for selection of peptide aptamers and the peptide aptamers selected from the libraries. FIG. 5A shows that the CDR3 region of the VH-DiF scaffold (PDB ID: 7D8B) was selected for replacement by rationally designed loops. The engineered loops were designed to present the EE-02 motif in the correct conformation to interact with eIF4E using polyGly linkers. However, the VH-DiF derived proteins, when tested, exhibited no binding to eIF4E. Peptide Epitope Loop Exchange (PELE) libraries were also constructed and inserted at the same site in the VH-DiF scaffold. Optimal linkers needed to present the EE-02 motif correctly for binding were selected by YSD (Yeast Surface Display). The YSD (yeast surface display) selection against eIF4E went through an initial round of selection performed with IMACs, followed by 2 rounds of in-solution selection using flow cytometry to enrich the population for high affinity eIF4E binders, where biotinylated eIF4E was detected using dye-labelled streptavidin. Insets show the enrichment in eIF4E cap-binders in the PELE library after rounds 2 and 3 of FACs selection. Negative control experiments were performed with the same library inputs that showed negligible non-specific binding within the enriched populations in the absence of eIF4E. FIG. 5B shows that the samples from the final round input for YSD selection were co-incubated with either m⁷GTP, 4E-BP1^{4ALA} or VH-M4 in order to compete with the VH-DiF population enriched for eIF4E binding with biotinylated eIF4E (measured in FIG. 5A). A significant reduction in the enriched population interacting with eIF4E only occurred with m⁷GTP treatment indicating that the selected eIF4E binders were specific for the cap-binding site. FIG. 5C shows the table that lists the 10 unique VH-DiF sequences identified from the 34 yeast clones sequenced in the final round of YSD selection, with their corresponding frequencies. A recognition motif was generated from the identified sequences using MEME (XXX), which in addition to showing the invariant cyclic peptide interaction motif, also identified that proline was preferentially enriched for at the position immediately preceding the motif. FIG. 5D shows the complex structure of eIF4E with VH-DiF^{CAP-01} (SEQ ID NO: 4) highlighting the binding of the PELE selected motif presenting linkers to eIF4E. The 2Fₒ-F_{c} electron density map of the cap interacting loop structure is shown in blue (1.2σ). FIG. 5E shows the overlay of the cap binding motif of VH-DiF^{CAP-01} (E¹⁰³MGFF¹⁰⁷) with the equivalent residues in EE-02 highlighting the loss of the water mediated interactions between EE-02 and E-103 and a small conformation change in E103, where the interaction with R112 and the structured water network are retained. However, it does result in an additional interaction with K162 not observed in the eIF4E:EE-02 complex. FIG. 5F shows that the cap binding motif of VH-DiF^{CAP-01} (E¹⁰³MGFF¹⁰⁷,) forms a similar β-hairpin-like structure to that seen in the eIF4E:EE-02 complex. Additionally, the two intra backbone hydrogen bonds that formed to stabilize the bound structure of the EE-02 cyclic peptide (FIG. 5C) are also observed in the VH-DiF^{CAP-01} complex with eIF4E. FIG. 5G shows that the hydrogen bond formed between C01 of EE-02 with R157 is not observed in the eIF4E:VH-DiF^{CAP-01} complex, where it is replaced with a hydrogen bond between P102 (white) of the ¹⁰⁰PLP¹⁰² linker and N155. FIG. 5H shows that the PELE selected linker (T¹⁰⁸NIPAMV¹¹⁴) form 3 hydrogen bonds with eIF4E: Residues T108 and Q109 form 2 hydrogen bonds with the indole group of W102 of eIF4E (3.7 Å and 3.1 Å, respectively), and a hydrogen bond forms between the amide and carbonyl groups of A112 and eIF4E's A204, respectively. FIG. 5I shows that residues 1110 and A113 of the linker region (T¹⁰⁸NIPAMV¹¹⁴) form multiples hydrophobic contacts with residues T203, A204, T206 and G208 in eIF4E, which stabilize the α-helical secondary structure of the eIF4E region 201 to 205. FIG. 5J shows that the conformation of E¹⁰³MGFF¹⁰⁷ is stabilized by a hydrophobic cluster principally formed by 1110 from the linker region (T¹⁰⁸NIPAMV¹¹⁴) and a salt bridge between VH-DiF^{CAP-01} (SEQ ID NO: 4) residues R51 and D36, which also interact with two buried structured waters. The water network in conjunction with R51 form hydrogen bonds with the polypeptide backbone of the PELE selected loop, helping to stabilize the conformation of the cap-site interaction motif for eIF4E binding.

FIG. 6 shows the analysis of immunoprecipitation (IP) pull down assays of the identified peptide aptamers. FIG. 6A shows that the Anti-FLAG IP pull down of HEK293 cells transfected with either VH-DIF^{CAP-01} (SEQ ID NO: 4), VH-DIF^{CAP-02} (SEQ ID NO: 5), VH-DIF^{CAP}-Cntrl (SEQ ID NO: 84) and VH-S4, a VH-domain that interacts with eIF4E at the eIF4G binding interface. IP experiments were performed 24 hours post transfection. Whole cell lysate (WCL) was also blotted for the corresponding proteins and is shown on the left of the blot. FIG. 6B shows that m⁷GTP pulldown of eIF4E containing complexes from HEK293 transfected with VH-DIF^{CAP-01}, VH-DIF^{CAP-02}, VH-DIF^{CAP-01} MA (M104A) (SEQ ID NO: 102) and VH-S4 (SEQ ID NO: 101). Whole cell lysate (WCL) was also blotted for the corresponding proteins and is shown on the left of the blot. In the blot below an equivalent pull-down was performed but with the HEK293 cells transfected with increasing amounts of expression vector. FIG. 6C shows that the HEK293 cells were transfected with either empty vector, VH-DIF^{CAP}01 or VH-DIF^{CAP-01} MA (M104A), and eIF4E phosphorylation and cyclin D1 expressions levels assessed via western blot. Actin was used as a loading control, whilst anti-FLAG was used to assess expression of the transfected proteins. Protein levels were assessed 48 hours post transfection. FIG. 6D shows that a bicistronic luciferase reporter, which measures the relative amount of cap-dependent translation (Renilla) to cap-independent translation (Firefly), was co-transfected with either empty vector (MOCK) or increasing amount of VH-DIF^{CAP-01} (SEQ ID NO: 4), VH-DIF^{CAP-01} MA, VH-S4 plasmid vector into HEK293 cells (see materials and methods). Renilla and Firefly luciferase activity was measured 48 h post transfection and plotted as a ratio-metric value. FIG. 6E shows that the Anti-His IP pulldown of purified VH-DIF^{CAP-01} (SEQ ID NO: 4) exogenously added to HEK293 cell lysates either treated with CGP57380 or vehicle control. Input lysate is shown on left hand side of the western blot.

FIG. 7 shows the protein-protein interaction assays in live cells. FIG. 7A shows the inset showing how the interaction of proteins A and B fused to SmBiT and LgBiT (Promega, USA) enables reconstitution of the active NanoBit (Promega, USA) luciferase. Graph shows the reconstituted luminescence activity of the various combinations of either eIF4E or VH-DIF^{CAP-01} fused at either the N- or C-terminal of SmBiT and LgBiT, respectively, co-transfected into HEK293 cells. Individual N- and C-terminal LgBiT-linked eIF4E and VH-DIF^{CAP-01} constructs co-transfected with SmBiT-HALO served as negative controls. FIG. 7B shows that to validate the specificity of the SmBiT-VH-DIF^{CAP-01} and LgBiT-eIF4E interaction pair, two VH-DIF^{CAP}-01 point mutant controls were generated (E103A (EA) (SEQ ID NO: 103) and M104A (MA) (SEQ ID NO: 102), respectively) and co-transfected into HEK293 cells with LgBiT eIF4E, which resulted in loss of bioluminescence. Inset: Cell samples replicating the NanoBiT experimental conditions were assessed for their relative levels of LgBiT fused eIF4E to endogenous eIF4E, and expression levels of the various SmBiT-VH-DIF^{CAP-01} constructs. FIG. 7C shows that (Right hand graph) the ability of the SmBiT-VH-DIF^{CAP-01}: LgBiT-eIF4E (termed NanoBIT^{CAP}) interaction pair to discriminate between different classes of eIF4E binders was tested by co-expressing it with either VH-S4 (a VH domain that interacts specifically with the eIF4G interaction site) or VH-DIF^{CAP-01} not fused to SmBiT, where only VH-DIF^{CAP-01} caused a decrease in luminescence. (Left hand graph) The specificity of VH-DIF^{CAP-01} was further investigated by co-expressing either VH-DIF^{CAP-01} or VH-S4 with the NanoBit eIF4E:eIF4G⁶⁰⁴⁻⁶⁴⁶ system, which measures binding at the eI4G interface and demonstrated that VH-DIF^{CAP-01} only interacts with the cap-binding interface.

FIG. 7D shows that HEK293 cells were transfected with the NanoBIT^{CAP} system and permeabilized with a sub-CMC (critical micelle concentration) concentration of digitionin. Cells were then treated with different titrations of small molecules that either specifically targeted the cap (m⁷GTP, m⁷GDP) or eIF4G (4EGI) binding interfaces of eIF4E. FIG. 7E shows that HEK293 cells were transfected with the NanoBit eIF4E:eIF4G⁶⁰⁴⁻⁶⁴⁶ system and again permeabilized with a sub-CMC (critical micelle concentration) concentration of digitionin. Cells were then treated with titration of the following compounds (m⁷GTP, m⁷GDP and 4EGI) to assess the specificity of the NanoBIT^{CAP} system.

FIG. 8 shows the binding energy decomposition analysis from MD simulations of eIF4E:EE-02 and eIF4E:VH-DIF^{CAP-01} complex structure. FIG. 8A shows MD simulations of the eIF4E:EE-02 complex structure which demonstrates that M4, F6 and F7 contribute a significant proportion of the binding energy of the complex, and FIG. 8B shows MD simulations of the eIF4E:VH-DIF^{CAP-01} complex showing that the EE-02 motif also underpins the energetics of the VH domain's interaction with eIF4E. The enlarged portion of the graph details the precise contributions being made by each residue of the interaction motif to the binding energy. Residues F106, F107 and M104 (equivalent to M4, F6 and F7 in EE-02) contribute significantly to the energetics of the complex.

FIG. 9 shows the structural deviation between bound and free states of VH-DIF^{CAP-01} and EE-02. FIG. 9A shows the averaged free state EE-02 structure, derived from MD simulations of the cyclic peptide alone, overlaid with the bound structure of EE-02 from the eIF4E:EE-02 crystal structure. FIG. 9B shows the RMSD plot of the MD simulation frames of unbound EE-02 against the bound crystal form. FIG. 9C shows the averaged free state VH-DIF^{CAP-01} structure, derived from MD simulations of the VH domain alone, overlaid with the eIF4E bound structure of VH-DIF^{CAP-01} from the crystal structure. The RMSD values sample a broad range inferring that the EE-02 peptide adopts a fold similar to the bound form, but is relatively flexible. FIG. 9D shows the RMSD plot of the MD simulation frames of unbound VH-DIF^{CAP-01} against the bound VH domain crystal form.

FIG. 10 shows the His-Tagged VH constructs (R1, R2, R3) with the cap-site binding peptide motif rationally grafted on at alternate positions (see insert) in the CDR3 loop were screened in a pull-down assay against glutathione beads with bound GST-tagged eIF4E. The left-hand panel shows the protein input into the assay, whilst the right-hand panel shows the results of the pull-down after stringent washing. VH-1C5 (SEQ ID NO: 99), a VH-domain that has been shown to interact at the eIF4E:4G interface was used as a positive control, whilst VH-1C5^{Scrambled} (SEQ ID NO: 104), where the corresponding CDR3 loop has been scrambled, was used as a negative control.

FIG. 11 shows the VH-DiF^{CAP} peptide aptamers identified by the yeast-based peptide epitope linker evolution experiments were tested for soluble expression in small scale bacteria cultures. His-tagged proteins were purified using Ni²⁺ chelated IMAC spin columns and analyzed using coomasie stained SDS PAGE gels to detect soluble protein. VH-DiF^{CAP-01} (SEQ ID NO: 4), VH-DiF^{CAP-05} (SEQ ID NO: 8) and VH-DiF^{CAP-09} (SEQ ID NO: 12) were selected for scaling up and further interaction analysis. The previously characterized VH domain (IC5), which interacts with eIF4E at the 4G binding site was used as a positive control.

FIG. 12 shows that VH-DiF^{CAP-01} also interacts weakly at a second site with eIF4E that is constituted from the CDR1 and CDR2 loops of the VH domain, where the residues S34 and S56 both form hydrogen bonds via their sidechains with the carbonyls of K52 and S53, respectively.

FIG. 13 shows protein structure of VH-DiF^{CAP-01} (SEQ ID NO: 4) bound to eIF4E and unbound VH-DiF^{CAP-01}. FIG. 13A shows the crystal structure of VH-DiF^{CAP-01} bound to eIF4E. Buried structured waters are depicted with spheres. The CDR3 loop bearing the ‘EMGFF’ cap-binding site interaction motif is highlighted in white. FIG. 13B shows the averaged structure of unbound VH-DiF^{CAP-01} (SEQ ID NO: 4) derived from MD simulations (see materials and methods). The CDR3 loop undergoes a structural relaxation, whereby the β-hairpin structure associated with the ‘EMGFF’ motif is lost and instead there is a general movement of the CDR3 loop away from the body of the VH domain scaffold. Interestingly, this movement is underpinned by significant structural changes in the hydrophobic core of the CDR3 loop structure with the L110 sidechain rotating out and the M113 sidechain rotating in to replace it. In association with these structural re-arrangements, the two buried structured water observed in the bound form (spheres) also adopt new position within the CDR3 loop, which help to stabilize the new conformation by forming 2 water mediated interactions (dashed lines) between the amide backbones of Q109 and G98 with the D36 sidechain, respectively.

FIG. 14 depicts the solvation properties of free and bound VH-DIF^{CAP-01}: FIG. 14A shows the solvation map depicting the averaged positions of the two buried waters, derived from the MD simulations of VH-DIF^{CAP-01} (SEQ ID NO: 4) in complex eIF4E, that stabilize the CDR3 loop conformation. FIG. 14B shows the graph depicting the number of water molecules observed at and within the vicinity of the 2 average water molecule position shown in FIG. 14A. FIG. 14C shows the solvation map depicting the averaged positions of the two buried waters, derived from the MD simulations of unbound VH-DIF^{CAP-01}, that change their position significantly in relationship to the bound form. FIG. 14D shows the graph depicting the number of water molecules observed at and within the vicinity of the 2 average water molecule position shown in FIG. 14C.

FIG. 15 shows the protein structures of VH-DIF^{CAP-01} (SEQ ID NO: 4) when bound with different target proteins. FIG. 15A shows that the CDR3 loop of VH-DiF^{CAP-01} folds back onto the former light-chain interaction surface of the VH domain. FIG. 15B shows that NanoBodies (VHH domain derived from a camelid antibody) in complex with lysozyme (PDB ID: 1Z4H) and FIG. 15C shows that RNase A (PDB ID: 2P4A) showing the interacting CDR3 loops folding back onto the main body of the VHH domain. Nanobodies in complex with β2 adrenoceptor (adrenoceptor-PDB ID: 3P0G) in FIG. 15D and GFP (PDB ID: 3K1K) in FIG. 15E, where the CDR3 interacting loops form no packing interactions with the VHH domains themselves.

FIG. 16 shows protein structures of VH-DiF^{CAP-01} (SEQ ID NO: 4) bound to eIF4E and VH domain interaction with VEGFA. FIG. 16A show that the CDR3 loop of VH-DiF^{CAP-01} folds back onto the former light-chain interaction surface, where the D38: R51 salt-bridge is located. The interaction of the CDR3 loop with the salt-bridge stabilizes its conformation enabling it to engage the cap-binding site on eIF4E. FIG. 16B show that this type of interaction does differ significantly from the reported VH domain interaction with VEGFA (PDB ID: 3P9W), where both the CDR3 and the former light-chain interaction surface are involved in macromolecular recognition. It also must be noted the CDR3 loop does not fold back on to the VH domain.

FIG. 17 shows the in vitro binding assay for peptide aptamers for eIF4A. FIG. 17A shows the dissociation rate constant (K_{d}) of the interaction between peptide aptamers (4AM3 (SEQ ID NO: 29), 4AM14 (SEQ ID NO: 39) and 4AM20 (SEQ ID NO: 61)) and eIF4A measured by Surface Plasmon Resonance in PBST running buffer. FIG. 17B shows the polypeptide sequence of peptide aptamers (4AM3 (SEQ ID NO: 29), 4AM14 (SEQ ID NO: 39) and 4AM20 (SEQ ID NO: 61)) and the peptide motif is “WXXSRTPW” as outlined. The peptide aptamer 4AM20 forms an expected disulphide bond as it was sensitive to reducing condition. FIG. 17C shows the dissociation rate constant of the interaction between peptide aptamers (4AM3 (SEQ ID NO: 29), 4AM14 (SEQ ID NO: 39) and 4AM20 (SEQ ID NO: 61)) and eIF4A measured by Surface Plasmon Resonance in PBST with 1 mM DTT.

FIG. 18 shows the binding assay between peptide aptamers to eIF4A. FIG. 18A shows the K_{d} of the interaction between peptide aptamer (4AM14 (SEQ ID NO: 39)) and eIF4A measured by Isothermal Titration calorimetry with and without TCEP. FIG. 18B shows the K_{d} of the interaction between peptide aptamer (4AM20 (SEQ ID NO: 61)) and eIF4A measured Isothermal Titration calorimetry in the presence of TCEP.

FIG. 19 shows the overexpression and crystallization of peptide aptamers with PCNA. FIG. 19A shows the polypeptide sequences of the peptide aptamers identified with binding affinity to PCNA. FIG. 19B shows the K_{d} of the binding affinity between the peptide aptamers and PCNA determined by competitive fluorescence polarization experiments.

FIG. 20 shows the expression of the peptide aptamers in mammalian cells and interaction between the peptide aptamers and PCNA. FIG. 20A shows the expression of peptide aptamers in HEK293 cells and the interaction of the peptide aptamers and PCNA in an anti-FLAG immunoprecipitation pull down assay. The peptide aptamers (VH-D1 (SEQ ID NO: 21) and VH-31 (SEQ ID NO: 19)) interact with PCNA as observed in the western blot analysis using anti-PCNA antibody. FIG. 20B shows the expression of peptide aptamers in A375 stable cell lines and the interaction of peptide aptamers (VH-D1 (SEQ ID NO: 21) and VH-31 (SEQ ID NO: 19)) and PCNA in an anti-FLAG immunoprecipitation pull down assay.

FIG. 21 shows the percentage of confluency of the cells in the presence of peptide aptamers for PCNA with and without doxycycline (DOX) The peptide aptamers (VH-D1 (SEQ ID NO: 21) and VH-31 (SEQ ID NO: 19)) inhibited proliferation in the stable transfected inducible cell lines.

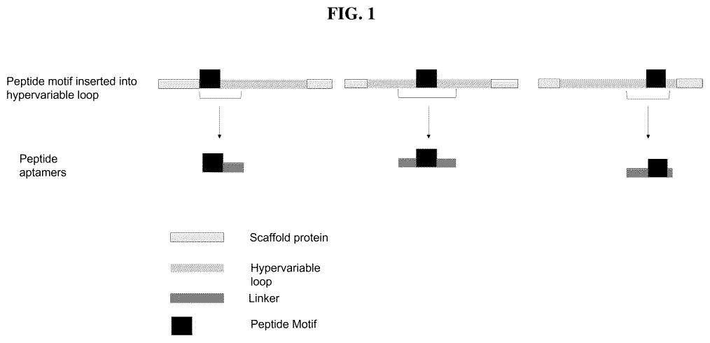

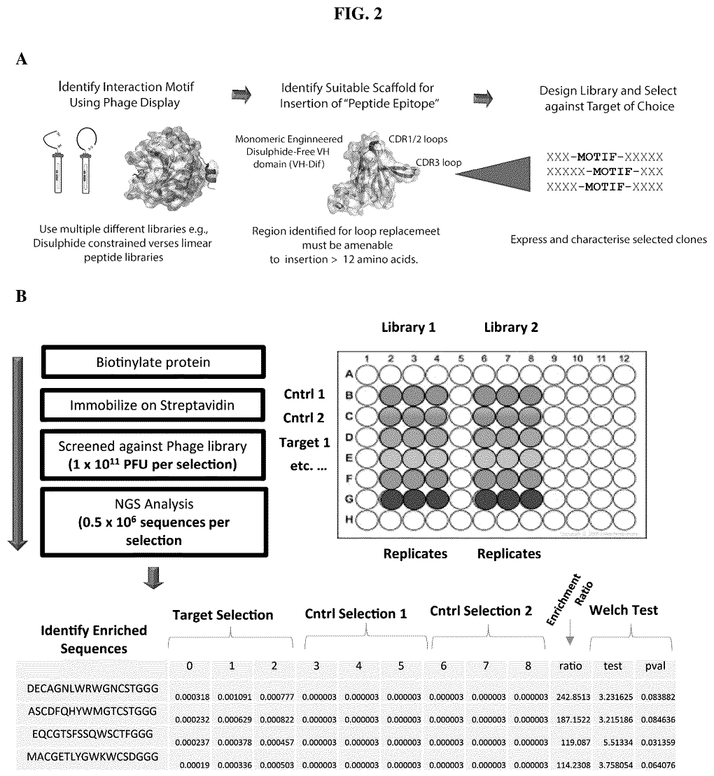

### DETAILED DESCRIPTION OF THE PRESENT INVENTION

In a first aspect, the present invention refers to a method of identifying and isolating a peptide aptamer (PA) that is capable of binding to a target protein comprising:
a) identifying a peptide motif that interacts with the target protein from a library of peptides;
b) inserting a hypervariable region in a scaffold protein;
c) inserting the peptide motif into a plurality of positions in the hypervariable region of the scaffold protein to generate one or more library of peptide aptamers, wherein the hypervariable region forms one or more linkers between the peptide motif and the scaffold protein;
d) contacting the library from step c with the target protein to identify the peptide aptamer that is capable of binding to the target protein; and
e) isolating the peptide aptamer from step d.

The identified peptide aptamer may be further engineered via maturation or chemical modifications to increase the binding affinity to the target protein. Maturation of the peptide aptamer may be post-translational modifications of the peptide aptamers. Post-translational modifications may include phosphorylation, acetylation, hydroxylation and methylation. Chemical modifications may be the addition of chemical groups within the peptide aptamer or at each end of the peptide aptamer. The chemical groups may include biotin, thiol, amide, carboxyl, linear or branched alkyl, lipids, fatty acids. For example, an amide group may be added at N-terminal and/or C-terminal of the peptide aptamer or within the peptide aptamer.

In one example, the library of peptides includes but is not limited to a phage library, a mRNA display library, a bacterial display library, a synthetic peptide library or combinations thereof. In a preferred example, the library of peptides is a phage library. The phage library may be a linear peptide phage library, a constrained peptide phage library or dodecapeptide library. It will generally be understood that a linear peptide phage library comprises a library of linear peptides or peptide motifs and the constrained peptide phage library comprises a library of peptides or peptide motifs that are structurally constrained. The peptide phage library may comprise a library of peptides constrained with a disulphide bond ranging from 4-mer to 12-mer. The peptides may be 4-mer, 5-mer, 6-mer, 7-mer, 8-mer, 9-mer, 10-mer, 11-mer or 12-mer. In some examples, the linear peptide phage library may comprise linear peptides or linear peptide motifs ranging from 7-mer to 12-mer. The linear peptides or peptide may be 7-mer, 8-mer, 9-mer, 10-mer, 11-mer or 12-mer. The linear peptides or peptide motifs adopt the conformation when binding to the target protein and the constrained peptides of peptide motifs adopt the conformation prior to binding to the target protein. The constrained peptide phage library includes but is not limited to a disulphide constrained peptide phage library, a cysteine constrained peptide phage library and an a-helical constrained peptide phage library. It will also be generally understood that a dodecapeptide phage library comprises a library of dodecapeptides (12-mer) or dodecapeptide motifs. One or more libraries in various combinations may be used in the methods of the invention. In one example, the phage library is a constrained peptide phage library. In a preferred example, the constrained peptide phage library is a disulphide constrained peptide library.

The method of isolating a peptide aptamer of the invention also comprises the step of inserting a hypervariable region into a scaffold protein. The hypervariable region is inserted into the loop of the scaffold protein. The hypervariable region may be inserted into any loop of any protein scaffold. The hypervariable region may be inserted into the protein scaffold using conventional molecular biology techniques. The conventional molecular biology techniques comprise restriction enzyme digestion, double stranded DNA cassette ligation and overlapping polymerase chain reaction techniques. The insertion of the hypervariable region into the protein scaffold is randomized which results in the isolation of a peptide aptamer and the peptide aptamer may be synthesized.

The identified peptide motif is inserted in a plurality of positions in the hypervariable region of the scaffold protein to generate one or more libraries of peptide aptamers comprising the peptide motif and one or more linkers derived from the hypervariable region. Each library may comprise peptide aptamers that comprise the peptide motif and linkers with identical number of amino acid residues at each of the C- and N-terminal. Each of the library may also comprises peptide aptamers that comprise peptide motif and linkers with different number of amino acid residues at each of the C- and N-terminal. For example, one library may comprise peptide aptamers comprising the peptide motif, and linkers comprising 5 amino acid residues long at the C-terminal and 5 amino acid residues long at the N-terminal. In another example, one library may comprise peptide aptamers comprising the peptide motif, and linkers comprising 5 amino acid residues at the C-terminal and 5 amino acid residues at the N-terminal, and peptide aptamers comprising peptide motif, and linkers comprising 7 amino acid residues at the C-terminal and 3 amino acid residues at the N-terminal. In another example, one library may comprise peptide aptamers comprising the peptide motif, and linkers comprising 10 amino acid residues at the C-terminal and 0 amino acid residues at the N-terminal and peptide aptamers comprising peptide motif, and linkers comprising 0 amino acid residues at the C-terminal and 10 amino acid residues at the N-terminal.

The peptide motif may be inserted randomly in one or more positions in the hypervariable region of the scaffold protein to generate one or more library of peptide aptamers. As such, the peptide motif may be inserted in one position, two positions, three positions, four positions, five positions, six positions, seven positions, eight positions in the hypervariable region of the scaffold protein. For example, the peptide motif may be inserted at three different positions to generate three different libraries of peptide aptamers. The peptide motif may be inserted at five different positions to generate five different libraries of peptide aptamers. In another example, the peptide motif may be inserted at three different positions to generate one library of peptide aptamers. The peptide motif may be inserted in six different positions to generate two libraries of peptide aptamers. The peptide motif may be inserted in five different positions to generate one library of peptide aptamers.

In some examples, the hypervariable region forms the linker sequences at the C-terminal and/or N-terminal of the peptide motif. The linker sequence may be located at the C-terminal, or the N-terminal, or both C- and N-terminals of the peptide motif. In one example, the linker sequence is from 0 to 10 amino acid residues long. The one or more linker sequences may be 1 amino acid residue long, 2 amino acid residues long, 3 amino acid residues long, 4 amino acid residues long, 5 amino acid residues long, 6 amino acid residues long, 7 amino acid residues long, 8 amino acid residues long, 9 amino acid residues long and 10 amino acid residues long. In some examples, the entire sequence of the hypervariable region may be located at the C-terminal of the peptide motif and the linker sequence at the N-terminal of the peptide motif may not be present. The entire sequence of the hypervariable region may be located at the N-terminal of the peptide motif and the linker sequence at the C-terminal of the peptide motif may not be present. The length of the linker sequences may affect the stability of the scaffold protein. The longer the linker sequences, the weaker the stability of the scaffold protein, thus affecting the sampling of the library.

In some examples, the linker sequence may be 3 amino acid residues long at the C-terminal and 7 amino acid residues long at the N-terminal. The linker sequence may be 5 amino acid residues long at the C-terminal and 5 amino acid residues long at the N-terminal. The linker sequence may be 7 amino acid residues long at the C-terminal and 3 amino acid residues long at the N-terminal. The linker sequence may be 2 amino acid residues long at the C-terminal and 8 amino acid residues long at the N-terminal. The linker sequence may be 6 amino acid residues long at the C-terminal and 4 amino acid residues long at the N-terminal. The linker sequence may be 10 amino acid residues long at the C-terminal with no linker sequence at the N-terminal. The linker sequence may be absent at the C-terminal and the linker sequence may be 10 amino acid residues long at the N-terminal.

It will generally be understood that the amino acid residues of the linker sequences are randomized based on where the peptide motif is inserted within the hypervariable region.

In one example, the hypervariable region is linked to the scaffold protein in a stable confirmation. The stable interaction between the hypervariable region and the scaffold protein allows the peptide motif inserted within the hypervariable region linked to be stably presented to a target protein for binding.

In one example, the scaffold protein includes but is not limited to VH domain, stefin-A and fibronectin. In a preferred example, the scaffold protein is the VH domain.

In one example, the library of peptide aptamers is a yeast surface display library. The yeast surface display library may be but not limited to the S. cerevisiae yeast surface display strain EBY100.

In one example, the peptide aptamer binds to the target protein at a binding affinity measured using a competition binding assay that measures a dissociation constant. The measurement of a dissociation constant defines the binding affinity of the peptide aptamer to the target protein. In one example, the peptide aptamer binds to the target protein with a dissociation constant of less than about 1000 nM, less than about 900 nM, less than about 800 nM, less than about 700 nM, less than about 600 nM, less than about 500 nM, less than about 400 nM, less than about 300 nM, less than about 200 nM, less than about 100 nM, less than about 50 nM. In a preferred example, the peptide binds to the target protein with a dissociation constant of less than about 100 nM.

In another aspect, the present invention refers to a peptide aptamer comprising an amino acid sequence selected from the group consisting of:

i)
(SEQ ID NO: 1)
X₁X₂X₃X₄X₅EMGFFX₆X₇X₈X₉X₁₀,
ii)
(SEQ ID NO: 2)
X₁₁X₁₂X₁₃EMGFFX₁₄X₁₅X₁₆X₁₇X₁₈X₁₉X₂₀,
and
iii)
(SEQ ID NO: 3)
X₂₁X₂₂X₂₃X₂₄X₂₅X₂₆X₂₇EMGFFX₂₈X₂₉X₃₀;

wherein X₁₋₃₀ is any amino acid;
wherein X₁₋₅, X₁₁₋₁₃ and X₂₁₋₂₇ comprise amino acid sequences for a first linker;
wherein X₆₋₁₀, X₁₄₋₂₀ and X₂₈₋₃₀ comprise amino acid sequences for a second linker;
optionally wherein X₅, X₁₃ and X₂₇ is an amino acid selected from the group consisting of proline (P), serine (S) and cysteine (C);
optionally wherein X₆, X₁₄ and X₂₈ is an amino acid selected from the group consisting of histidine (H), threonine (T), leucine (L), glutamine (Q), serine (S), lysine (K), alanine (A), glutamate (E) and valine (V); and
optionally where in X₇, X₁₅ and X₂₉ is an amino acid selected from the group consisting of asparagine (N), valine (V), threonine (T), aspartate (D), cysteine (C), proline (P) and leucine (L)
wherein the peptide aptamer binds to eIF4E.

In one example, the peptide aptamer binds to eIF4E in an open conformation. The nucleotide or the cap of the peptide motif of the peptide aptamer is capable of binding to eIF4E. The term “open conformation” refers to the swinging of W56 and W102 out of the cap-binding site. In contrast, the term “closed conformation” refers to the stacking of W56 and W102 in parallel on either side of the guanine moiety when W56 and W102 have swung back into the cap binding site.

In one example, the peptide aptamer is linked to the scaffold protein in a stable conformation. The peptide aptamer linked to the scaffold protein presents the peptide motif binding to eIF4E in a stable conformation. The stable conformation in the context of binding to eIF4E refers to the interaction of R51 and D36 two structured water molecules and the backbone of the CDR3 loop of the hypervariable region. Presentation of the peptide motif in stable conformation allows the high affinity binding of the peptide motif to eIF4E.

In one example, the peptide aptamer binds to eIF4E at the mRNA 5′ binding site. The peptide aptamer binds to eIF4E at the mRNA 5′ binding site with high affinity. The peptide aptamer binds to eIF4E at the mRNA 5′ binding site with a dissociation constant (Kc) of less than about 1000 nM, less than about 900 nM, less than about 800 nM, less than about 700 nM, less than about 600 nM, less than about 500 nM, less than about 400 nM, less than about 300 nM, less than about 200 nM, less than about 100 nM, less than about 50 nM. In one example, the peptide aptamer binds to eIF4E at the mRNA 5′ binding site with a Kc of less than 50 nM.

In one example, the peptide aptamer binding to eIF4E inhibits cap-dependent translation.

In one example, the peptide aptamer comprises the amino acid sequence selected from the group consisting of SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13. In a preferred example, the peptide aptamer comprises the amino acid sequence set forth in SEQ ID NO: 4.

In another aspect, the present invention refers to a peptide aptamer comprising an amino acid sequence X₁X₂X₃X₄X₅X₆X₇YPMFX₈X₉X₁₀ (SEQ ID NO:14);
wherein X₁-X₁₀ is any amino acid;
wherein X₁₋₇ comprises amino acid sequences for a first linker;
wherein X₈₋₁₀ comprises amino acid sequences for a second linker;
optionally wherein X₁ is serine (S);
optionally wherein X₂ is an amino acid selected from the group consisting of proline (P), glutamine (Q), tyrosine (Y), histidine (H) and arginine (R);
optionally wherein X₃ is an amino acid selected from the group consisting of glycine (G), valine (V), serine (S), arginine (R) and threonine (T);
optionally wherein X₄ is an amino acid selected from the group consisting of threonine (T), asparagine (N), arginine (R), serine (S), proline (P) and glutamate (E);
optionally wherein X₅ is an amino acid selected from the group consisting of histidine (H), proline (P), valine (V), aspartate (D), threonine (T) and arginine (R);
optionally wherein X₆ is an amino acid selected from the group consisting of proline (P), leucine (L) and isoleucine (I);
optionally wherein X₇ is an amino acid selected from the group consisting of lysine (K), valine (V), leucine (L) and phenylalanine (F);
optionally wherein X₈ is an amino acid selected from the group consisting of histidine (H), valine (V), isoleucine (I) and serine (S);
optionally wherein X₉ is an amino acid selected from the group consisting of leucine (L), asparagine (N), arginine (R) and proline (P); and
optionally wherein X₁₀ is an amino acid selected from the group consisting of arginine (R), histidine (H) and tryptophan (W),
wherein the peptide aptamer binds to proliferating cell nuclear antigen (PCNA).

In one example, the peptide aptamer comprises the amino acid sequences selected from the group consisting of SEQ ID NO: 15, SEQ ID NO: 16, SEQ ID NO: 17, SEQ ID NO: 18, SEQ ID NO: 19, SEQ ID NO: 20, SEQ ID NO: 21 and SEQ ID NO: 22. In a preferred example, the peptide aptamer comprises the amino acid sequence set forth in SEQ ID NO: 19 or SEQ ID NO: 21. In another preferred example, the peptide aptamer comprises the amino acid sequence set forth in SEQ ID NO: 23.

In one example, the peptide aptamer binding to PCNA inhibits cell proliferation. The peptide aptamer binding to PCNA inhibits cell proliferation in stable transfected inducible cell lines. Examples of stable transfected inducible cell lines include but not limited to HEK293 cells and A375 stable cell lines.

In another aspect, the present invention refers to a peptide aptamer comprising an amino acid sequence selected from the group consisting of

i)
(SEQ ID NO: 24)
X₁X₂X₃X₄X₅WX₆X₇SRTPWX₈X₉X₁₀X₁₁X₁₂,
ii)
(SEQ ID NO: 25)
X₁₃X₁₄X₁₅X₁₆X₁₇X₁₈X₁₉WX₂₀X₂₁SRTPWX₂₂X₂₃X₂₄,
and
iii)
(SEQ ID NO: 26)
X₂₅X₂₆X₂₇WX₂₈X₂₉SRTPWX₃₀X₃₁X₃₂X₃₃X₃₄X₃₅X₃₆;

wherein X₁-X₃₆ is any amino acid;
wherein X₁₋₅, X₁₃₋₁₉ and X₂₅₋₂₉ comprise amino acid sequences for a first linker;
wherein X₈₋₁₂, X₂₂₋₂₄ and X₃₀₋₃₆ comprise amino acid sequences for a second linker;
optionally wherein X₁ and X₁₅ is an amino acid selected from the group consisting of leucine (L), glutamine (Q), arginine (R), valine (V), tyrosine (Y), glycine (G), alanine (A) and threonine (T);
optionally wherein X₂ and X₁₆ is an amino acid selected from the group consisting of threonine (T), arginine (R), serine (S), proline (P), alanine (A) and isoleucine (I);
optionally wherein X₃, X₁₇ and X₂₅ is an amino acid selected from the group consisting of tryptophan (W), leucine (L), alanine (A), isoleucine (I), serine (S), threonine (T), glutamine (Q), asparagine (N), glutamate (E), cysteine (C), proline (P), glycine (G), valine (V), arginine (K), arginine (R), phenylalanine (F), and methionine (M); optionally wherein X₄, X₁₈ and X₂₆ is an amino acid selected from the group consisting of threonine (T), histidine (H), glutamine (Q), leucine (L), glutamate (E), valine (V), glycine (G), arginine (R), phenylalanine (F), tyrosine (Y), serine (S), isoleucine (I), alanine (A), tryptophan (W) and cysteine (C);
optionally wherein X₅, X₁₉ and X₂₇ is an amino acid selected from the group consisting of arginine (R), tyrosine (Y), lysine (K), leucine (L), histidine (H), proline (P), serine (S), tryptophan (W) and valine (V);
optionally wherein X₆, X₂₀ and X₂₈ is an amino acid selected from the group consisting of valine (V), arginine (R), isoleucine (I), histidine (H), leucine (L), serine (S), phenylalanine (F), alanine (A), glycine (G), threonine (T) and lysine (K);
optionally wherein X₇, X₂₁ and X₂₉ is an amino acid selected from the group consisting of asparagine (N), phenylalanine (F), tryptophan (W), leucine (L), alanine (A), glycine (G), lysine (K), glutamate (E), serine (S), arginine (R), threonine (T), tyrosine (Y) and aspartate (D);
optionally wherein X₈, X₂₂ and X₃₀ is an amino acid selected from the group consisting of asparagine (N), histidine (H), phenylalanine (F), serine (S), tryptophan (W), alanine (A), threonine (T), tyrosine (Y), lysine (K), arginine (R), valine (V) and cysteine (C); optionally wherein X₉, X₂₃ and X₃₁ is an amino acid selected from the group consisting of valine (V), phenylalanine (F), arginine (R), isoleucine (I), asparagine (N), leucine (L), lysine (K), methionine (M), tryptophan (W), threonine (T), glycine (G) and alanine (A);
optionally wherein X₁₀, X₂₄ and X₃₂ is an amino acid selected from the group consisting of isoleucine (I), arginine (R), tyrosine (Y), leucine (L), valine (V), lysine (K), histidine (H), alanine (A), methionine (M), tryptophan (W), serine (S), threonine (T) and proline (P);
optionally wherein X₁₁ and X₃₃ is an amino acid selected from the group consisting of glycine (G), leucine (L), phenylalanine (F), arginine (R), tyrosine (Y), methionine (M), alanine (A), histidine (H), tryptophan (W), asparagine (N), lysine (K), serine (S), threonine (T) and glutamine (Q);
optionally wherein X₁₂ and X₃₄ is an amino acid selected from the group consisting of phenylalanine (F), threonine (T), histidine (H), arginine (R), tyrosine (Y), valine (V), aspartate (D), alanine (A), serine (S), leucine (L), proline (P), lysine (K) and tryptophan (W);
optionally wherein X₁₃ is an amino acid selected from the group consisting of asparagine (N) and leucine (L);
optionally wherein X₁₄ is an amino acid selected from the group consisting of valine (V), and leucine (L);
optionally wherein X₃₅ is an amino acid selected from the group consisting of (H), arginine (R), methionine (M), threonine (T), lysine (K), valine (V), leucine (L), serine (S), asparagine (N), isoleucine (I), aspartate (D) and glycine (G); and
optionally wherein X₃₆ is an amino acid selected from the group consisting of glutamine (Q), isoleucine (I), leucine (L), phenylalanine (F), proline (P), arginine (R), threonine (T), tyrosine (Y), aspartate (D), lysine (K), glycine (G), serine (S) and alanine (A);
wherein the peptide aptamer binds to eIF4A.

In one example, the peptide aptamer comprises the amino acid sequences selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60 and SEQ ID NO: 61.

In one example, the peptide aptamers of the present invention may not be constrained by a disulphide bond. The peptide aptamers may be engineered to remove the disulphide bonds from the constrained peptide motif and the disulphide bonds are replaced with linkers for increased stability of the peptide aptamers. Increased stability of peptide aptamers results in high affinity binding to the target protein. The peptide aptamers may be expressed in a cell to mimic the peptide motifs constrained by disulphide bonds. In another example, the peptide aptamers of the present invention may be constrained by a disulphide bond. The peptide aptamer comprises a cysteine amino acid in the linker sequence at each of the N-terminal and the C-terminal. The peptide aptamers constrained by a disulphide bond may be employed in diagnostic or purification applications to elute bound substrate using DTT.

In a preferred example, the peptide aptamer comprises the amino acid sequence set forth in SEQ ID NO: 29, SEQ ID NO: 39 and SEQ ID NO: 61.

In one example, the peptide aptamers of the present invention comprise a peptide motif and/or one or more linker sequences located in a hypervariable region of a scaffold protein.

In one example, the scaffold protein includes but is not limited to VH domain, stefin-A and fibronectin. In a preferred example, the scaffold protein is the VH domain.

In one example, the peptide aptamers of the present invention may be engineered and identified using the method as described herein. It should be clear to the person skilled in the art that the method as described herein can be used to engineer and identify peptide aptamers that bind to the target protein of interest and should not be restricted to the peptide aptamers of the present invention.

In another aspect, the present invention refers to the peptide aptamer as described herein for use as a medicament.

In another aspect, the present invention refers to a use of the peptide aptamer as described herein in the manufacture of a medicament for treating a condition associated with dysregulated cap-dependent translation, dysregulated DNA replication, dysregulated DNA repair and/or dysregulated mRNA translation.

In another aspect, the present invention refers to a method of treating a condition associated with dysregulated cap-dependent translation, dysregulated DNA replication, dysregulated DNA repair and/or dysregulated mRNA translation, comprising administering the peptide aptamer as described herein to a subject in need thereof. The peptide aptamer may in some examples be administered sequentially or simultaneously with one or more therapeutic agents. Examples of therapeutic agents include but are not limited to immunotherapy, chemotherapy or anti-viral therapy. In one example, the peptide aptamer is administered as a combinatorial treatment with immunotherapy. Immunotherapy may comprise therapies that downregulate the STAT1 pathway.

In one example, the present disclosure refers to the peptide aptamer as described herein for use in treating a condition associated with dysregulated cap-dependent translation, dysregulated DNA replication, dysregulated DNA repair and/or dysregulated mRNA translation.

In one example, dysregulated cap-dependent translation is a result of aberrant protein expression or aberrant protein activity. In one example, dysregulated cap-dependent translation is a result of aberrant eIF4E expression or aberrant eIF4E activity. In some examples, aberrant eIF4E expression is overexpression of eIF4E and aberrant eIF4E activity is an increase of eIF4E above the normal physiological levels. In one example, the condition associated with eIF4E overexpression is cancer.

In another example, the condition associated with dysregulated cap-dependent translation is a disease associated with a viral infection. In some examples, the virus invades the cell and drives cap-dependent translation. As a result, the cap-dependent translation may be increased or decreased as compared to the baseline activity.

In one example, dysregulated DNA replication, dysregulated DNA repair and dysregulated mRNA translation is also a result of aberrant protein expression or aberrant protein activity. For example, dysregulated DNA replication and dysregulated DNA repair is a result of overexpression of PCNA. In another example, dysregulated mRNA translation is a result of the aberrant eIF4A expression. Aberrant eIF4A expression refers to the overexpression of eIF4A and is an increase in eIF4A expression above the normal physiological levels.

In one example, the condition associated with dysregulated cap-dependent translation, dysregulated DNA replication, dysregulated DNA repair and/or dysregulated mRNA translation may include but is not limited to cancer, a disease associated with a viral infection and obesity. In one example, the cancer includes but is not limited to melanoma, triple negative breast cancer, lung cancer, colorectal cancer and prostate cancer. In a preferred example, the cancer is melanoma.

In another example, the disease associated with the viral infection is a disease caused by a virus. In one example, the virus is an RNA virus. Examples of the RNA virus include but are not limited to coronavirus, orthomyxovirus, rhabdovirus, reovirus, hantavirus and alphavirus.

In one aspect, the present invention refers to a method to identify a candidate peptide or nucleic acid that binds to a target protein in a live cell comprising:
a) fusing LargeBiT (LgBiT) to the target protein and fusing Small BiT (SmBiT) to a peptide aptamer that binds to the target protein, or fusing SmBiT to the target protein and fusing LgBiT to a peptide aptamer that binds to the target protein;
b) (i) expressing the fusion proteins from step a in the live cell; measuring the level of signal emitted in step b(i), wherein a signal is emitted when the target protein binds to the peptide aptamer; contacting the cell expressing the bound fusion protein with the candidate peptide or nucleic acid, optionally wherein the cell is permeabilized prior to contacting with the candidate peptide or nucleic acid; or
   (ii) expressing the fusion proteins from step a and the candidate peptide or nucleic acid in the live cell;
   measuring the level of signal emitted in step b(ii), wherein the signal is emitted when the target protein binds to the peptide aptamer; and
c) detecting a change in level of signal relative to a reference, wherein a decrease in the level of signal emitted is indicative of binding of the candidate peptide or nucleic acid to the target protein.

The candidate peptide may be an antagonist that binds to the cap-binding site or an antagonist that binds outside the cap-binding site. These antagonists may induce dissociation between the peptide aptamer and target protein.

In one example, the reference is a live cell which expresses the fusion proteins from step (a) and optionally a peptide or nucleic acid that does not bind to the fusion proteins from step (a) or a live cell which expresses the fusions proteins from step (a) and the candidate peptide or nucleic acid prior to binding to the target protein.

In one example, the live cell is a mammalian cell. In one example, the mammalian cell is a human cell. In a preferred example, the human cell is a human embryonic kidney (HEK) 293 cell.

The target protein may by any protein that interacts with the candidate peptide or nucleic acid in a live cell. In one example, the target protein includes but is not limited to eIF4E, PCNA and eIF4A.

The peptide aptamer fused to the SmallBiT (SmBiT) or LargeBiT (LgBiT) may be any peptide aptamer that binds to a target protein. In one example, the peptide aptamer fused to the SmallBiT (SmBiT) or LargeBiT (LgBiT) is the peptide aptamer as described here.

The candidate peptide or nucleic acid may be any peptide or nucleic acid that bind to a target protein. In one example, the candidate peptide is the peptide aptamer as described herein.

The methods as described herein may be utilized to identify any candidate peptide or nucleic acid that display binding affinity to a target protein of interest directly in live cells.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### EXPERIMENTAL SECTION

Non-limiting examples of the invention and comparative examples will be further described in greater detail by reference to specific Examples, which should not be construed as in any way limiting the scope of the invention.

### Materials and Methods

### Peptide Synthesis

Peptides were ordered from and synthesized by Mimotopes, Clayton, Australia. Peptides were purified using HPLC to >90% purity. All peptides were amidated at their C-terminus and acetylated at their N-terminus. Peptides were purified using HPLC to >90% purity.

eIF4E Expression and Purification for Crystallisation and Biophysical Assays

Full-length human eIF4E was expressed and purified as described below. Rossetta pLysS competent bacteria were transformed with the pET11d expression plasmid containing the full-length eIF4E clone. Cells expressing the full-length eIF4E construct were grown in LB medium at 37° C. to an OD600 of ˜0.6 and eIF4E induction was started with 1 mM IPTG. The culture was immediately placed in a shaker-incubator for 3 h at 37° C. Cells were harvested by centrifugation and the cell pellets were resuspended in 50 mM Tris pH 8.0, 10% sucrose, and were then sonicated. The sonicated sample was centrifuged for 10 min at 17,000 g at 4° C. The resulting pellet was resuspended in Tris/Triton buffer (50 mM Tris pH 8.0, 2 mM EDTA, 100 mM NaCl, 0.5% Triton X-100). The sample was then centrifuged at 25,000 g for 15 min at 4° C. and the pellet was resuspended in Tris/Triton buffer. After re-centrifugation, the remaining pellet was solubilised in 6 M guanidinium hydrochloride, 50 mM Hepes-KOH pH 7.6, 5 mM DTT. The protein concentration of the sample was then adjusted to 1 mg/mL. The denatured protein was refolded via a 1/10 dilution into refolding buffer consisting of 20 mM Hepes-KOH 7.6, 100 mM KCl and 1 mM DTT. The refolded protein was concentrated and desalted using a Amersham PD10 column into refolding buffer. The eIF4E protein sample was run over a monoQ column and eluted with a 1 M KCl gradient. eIF4E eluted as a sharp peak at a ˜ 0.3M KCl.

eIF4A Expression and Purification

eIF4A was cloned into the GST fusion expression vector pGEX-6P1 (GE Lifesciences). BL21 DE3 competent bacteria were then transformed with the GST-tagged fusion constructs. A single colony was picked and grown in LB medium at 37° C. to an OD600 of ˜0.6 and induction was carried out overnight with 0.3 mM IPTG at 16° C. Cells were harvested by centrifugation, and the cell pellets were resuspended in PBS (Phosphate Buffered Saline, 2.7 mM KCl and 137 mM NaCl, pH 7.4) and then sonicated. The sonicated sample was centrifuged for 60 min at 17,000 g at 4° C. The supernatant was applied to a 5 ml FF GST column (Amersham) pre-equilibrated in PBS buffer with 1 mM DTT. The column was then further washed by 6 volumes of PBS. Proteins were then purified from the column by either 1) cleavage with thrombin (Sigma-Aldrich) protease or 2) elution with glutathione. 1) Ten units of thrombin (Sigma-Aldrich) protease, in one column volume of PBS with 1 mM DTT buffer, were injected into the column. The cleavage reaction was allowed to proceed overnight at 4° C. The cleaved protein was then eluted off the column with wash buffer. Protein fractions were analysed with SDS page gel and concentrated using a Centricon (3.5 kDa MWCO) concentrator (Millipore). Protein samples were then dialyzed into a buffer solution containing 20 mM Tris pH 8.0 with 1 mM DTT and loaded onto a mono Q column pre-equilibrated in buffer A (20 mM Tris, pH 8.0, 1 mM DTT). The column was then washed in 6 column volumes of buffer A and bound protein was eluted with a linear gradient of 1 M NaCl over 25 column volumes. Protein fractions were analysed with SDS page gel and concentrated using a Centricon (3.5 kDa MWCO) concentrator (Millipore). The cleaved constructs were then purified to 90% purity. Protein concentration was determined using A280. 2) The column was then washed in 6 column volumes of PBS with 1 mM DTT and bound protein was eluted with a linear gradient of 50 mM Tris, pH 8.0, 0.1 M NaCl with 250 mM glutathione over 10 column volumes. Protein fractions were analysed with SDS page gel and concentrated using a Centricon (3.5 kDa MWCO) concentrator (Millipore). Concentrated protein was dialysed into PBS with 1 mM DTT and protein concentration determined using A280.

### PCNA Expression and Purification.

A plasmid containing the gene for human tagged to sumo and a hexa his tag was transformed into BL21-DE3 E. coli cells for protein expression. A single colony was picked and inoculated in 1L of LB medium. Upon reaching log phase, cells were induced with 1 mM isopropyl-β-D-thiogalactopyranoside and harvested after 3 h at 37° C. Pelleted cells were lysed in a buffer containing 25 mM HEPES, pH 7.5, 300 mM NaCl and 5 mM imidazole (buffer A). Cell lysates were clarified at 13 000 rpm for 45 min at 4° C. The supernatant was applied over a 0.22 μm filter and loaded onto a chelating column charged with 100 mM nickel sulfate (GE Healthcare). The column was washed with 20 CVs of buffer A and then 50 Unit of sumo was injected onto the detached column in cleavage buffer (25 mM HEPES pH 7.5, 300 mM NaCl, 20 mM imidazole, 1 mM DTT). Cleavage was performed overnight at 4° C. and then the column reattached and the cleaved PCNA eluted off. Fractions containing PCNA were pooled and buffer exchanged into 25 mM HEPES, pH 7.5, 100 mM NaCl and 1 mM DTT. These were then further purified using a superdex-75 size exclusion column and then concentrated.

eIF4E and 4EBP1^{4ALA} Mutants Expression and Purification for Sortase Labelling

eIF4E and 4EBP1^{4ALA} mutants were cloned into the GST fusion expression vector pGEX-6P1 (GE Lifesciences). BL21 DE3 competent bacteria were then transformed with the GST-tagged fusion constructs. A single colony was picked and grown in LB medium at 37° C. to an OD600 of ˜0.6 and induction was carried out overnight with 0.3 mM IPTG at 16° C. Cells were harvested by centrifugation, and the cell pellets were resuspended in PBS (Phosphate Buffered Saline, 2.7 mM KCl and 137 mM NaCl, pH 7.4) and then sonicated. The sonicated sample was centrifuged for 60 min at 17,000 g at 4° C. The supernatant was applied to a 5 ml FF GST column (Amersham) pre-equilibrated in PBS buffer with 1 mM DTT. The column was then further washed by 6 volumes of PBS. Proteins were then purified from the column by 1) cleavage with thrombin (Sigma-Aldrich) protease, 2) elution with glutathione or 3) cleavage with PreScission (GE Lifesciences) protease.

1) Ten units of thrombin (Sigma-Aldrich) protease, in one column volume of PBS with 1 mM DTT buffer, were injected into the column. The cleavage reaction was allowed to proceed overnight at 4° C. The cleaved protein was then eluted off the column with wash buffer. Protein fractions were analysed with SDS page gel and concentrated using a Centricon (3.5 kDa MWCO) concentrator (Millipore). Protein samples were then dialyzed into a buffer solution containing 20 mM Tris pH 8.0 with 1 mM DTT and loaded onto a mono Q column pre-equilibrated in buffer A (20 mM Tris, pH 8.0, 1 mM DTT). The column was then washed in 6 column volumes of buffer A and bound protein was eluted with a linear gradient of 1 M NaCl over 25 column volumes. Protein fractions were analysed with SDS page gel and concentrated using a Centricon (3.5 kDa MWCO) concentrator (Millipore). The cleaved constructs were then purified to 90% purity. Protein concentration was determined using A280.

2) The column was then washed in 6 column volumes of PBS with 1 mM DTT and bound protein was eluted with a linear gradient of 50 mM Tris, pH 8.0, 0.1 M NaCl with 250 mM glutathione over 10 column volumes. Protein fractions were analysed with SDS page gel and concentrated using a Centricon (3.5 kDa MWCO) concentrator (Millipore). Concentrated protein was dialysed into PBS with 1 mM DTT and protein concentration determined using A280.

3) Ten units of PreScission protease, in one column volume of PBS with 1 mM DTT buffer, were injected onto the column. The cleavage reaction was allowed to proceed overnight at 4° C. The cleaved protein was then eluted off the column with wash buffer. Protein fractions were analysed with SDS page gel and concentrated using a Centricon (3.5 kDa MWCO) concentrator (Millipore). Protein samples were then dialyzed into a buffer solution containing 20 mM Tris pH 8.0 with 1 mM DTT and loaded onto a mono Q column pre-equilibrated in buffer A (20 mM Tris, pH 8.0, 1 mM DTT). The column was then washed in 6 column volumes of buffer A and bound protein was eluted with a linear gradient of 1 M NaCl over 25 column volumes. Protein fractions were analysed with SDS page gel and concentrated using a Centricon (3.5 kDa MWCO) concentrator (Millipore). The cleaved constructs were then purified to 90% purity. Protein concentration was determined using A280.

### Sortase (SrtA^{8M}) Expression and Purification

The protein sequence corresponding to 61-206 of SrtA (Staphylococcus aureus) containing the following mutations (P94R, D160N, D165A, K190E, K196T, E105K, E108A and G167E) was ordered as a gene fragment from IDT (Integrated DNA technologies). The sequence was PCR amplified and inserted into a pNIC-CH bacterial expression plasmid via ligation independent cloning in frame with a C-terminal 6×His tag. The pNIC-CH-(61-206) SrtA^{8M} (termed SrtA^{8M}) expression vector was transformed into BL21(DE3) Rosetta competent cells and a single colony was used to inoculate a 20 ml starter culture in TB (terrific broth containing 25 ug/ml of chloramphenicol and 20 ug/ml of kanamycin), which was incubated overnight at 37° C. and shaken at 200 rpm. The starter culture was used to inoculate 750 ml of TB and was incubated at 37° C. until a O.D₆₀₀ reading of 2.0 was attained. Next, the temperature of the culture was lowered to 18° C. and protein expression induced with 0.5 mM of IPTG overnight. Cells were harvested by centrifugation, and the cell pellets were resuspended in 20 ml of lysis buffer (100 mM HEPES pH 8.0, 500 mM NaCl, 10 mM Imidazole, 10% glycerol, 0.5 mM TCEP, 1000 u Benzonase (Merck)) and then sonicated. The sonicated sample was centrifuged for 30 min at 17,000 g at 4° C. Supernatants were then filtered through 1.2 μm syringe filters and were loaded onto a Ni-nitrilotriacetic acid (NTA) column, pre-equilibrated with 20 mM HEPES pH 7.5, 100 mM NaCl, and 0.5 mM TCEP. The column was then washed with 5 column volumes of the same buffer containing 10 mM Immidazole. Hexahistidine tagged SrtA^{8M} was then eluted with a 1 M imidazole linear gradient. The protein was further purified by size exclusion chromatography (HiLoad 16/60 Superdex 75 prep grade, Cytiva Lifescience) using a 20 mM HEPES pH 7.5, 300 mM NaCl, 10% (v/v) glycerol, 0.5 mM TCEP buffer. Protein concentration was determined using A280 with an extinction coefficient determined from the primary sequence of the construct determined by ProtPARAM.

N-Terminal Biotin Labelling of eIF4E Mediated by SrtA^{8M}

Sortase-mediated ligation was used to specifically label eIF4E at the N-terminal with biotin. Cleavage of the GST-fused eIF4E with thrombin leaves a single glycine at the N-terminus. The ligation was carried out with thrombin cleaved eIF4E at 50 μM, SrtA^{8M} at 1 μM, and biotin-KGGGLPET-GG-OHse (Ac)-amide peptide at 200 μM in 200 μL of ligation buffer (50 mM Tris pH 8.0, 150 mM NaCl, 1 mM TCEP). SortaseA61-206/8M contains mutations that increase ligation efficiency and make it calcium-independent2. The ligation was incubated at room temperature for 4 hours. SrtA^{8M}, which contains a C-terminal 6×His-tag, was removed with Dynabeads His-Tag (cat #10104D, Thermo Fisher). The biotinylated protein was then dialyzed at 4° C. using slide-A-Lyzer cassette (10 k MWCO) against 2 L of an appropriate buffer. The buffer was changed after 4-5 hours and the dialysis was repeated overnight. The biotinylated protein was aliquoted, snap-frozen with liquid nitrogen, and stored at −80° C.

### Phage Display

An M13 phage library (Ph.D.-12, New England Biolabs) encoding random 12-mer peptides at the NH₂ terminus of pIII coat protein (2.7×10⁹ sequences) was used. Biotinylated full length eIF4E was loaded onto 10 μl of steptavdin M280 magnetic Dynabeads (Invitrogen). The loaded beads were incubated with blocking buffer (20 mM HEPES pH 7.6, 0.1 M KCL, 0.5% Tween20, 2% BSA) for 1 h at room temperature, washed with buffer W (20 nM HEPES pH 7.6, 0.1 M KCL, 0.5% Tween 20), and incubated in buffer W at room temperature with 4×10¹⁰ phages. Magnetic M280 beads were washed 8 times in buffer W. Bound phages were eluted with 0.2 M glycine (pH 2.2) and neutralized with 1 M Tris (pH 9.1). The eluted phages were amplified as instructed by the manufacturer. The selection process was repeated for three cycles. Phage plaques from the final round were picked and amplified as described by the manufacturer and sequenced.

M13 phage library (Ph.D.-C7C, New England Biolabs) encoding random 7-mer peptides flanked by two Cys was used. A 96-well microplate (Corning, #3370) was first coated with streptavidin (100 μg/mL) in 100 mM NaHCO₃(pH 8.4) at 4° C. overnight. After washing with 4×200 μL of binding buffer (50 mM Tris-HCl, 150 mM NaCl, 2 mM MgCl₂, pH 7.4), the wells were filled with the corresponding biotinylated protein (eIF4E, eIF4A or MDM2, 20 g/mL) in the binding buffer. After incubating at room temperature for 15 min, the microplate was washed with 4×200 μL of binding buffer and blocked with blocking buffer (binding buffer plus 1% BSA and 0.1% Tween-20) for 1 hour at room temperature. In parallel, the phage library was diluted to ˜1.0×10¹² pfu/mL in the blocking buffer. After removal of the blocking buffer from the microplate, phage library (100 μL per well) was added, incubated for 1 hour at room temperature, and washed with 4×200 μL of washing solution (binding buffer+0.1% Tween-20), followed by 2×200 μL washing solution containing 1 mM streptavidin, and finally with 4×200 μL of washing solution. The bound phages were eluted for 9 min with 0.2 M glycine (pH 2.2) plus 1% BSA and neutralized with 1 M Tris (pH 9.1). The eluted phages were amplified according to manufacturer's instruction and phage DNA was extracted by QIAprep spin Miniprep kit. The single-stranded DNA was then converted to Illumina-compatible double-stranded DNA amplicon by PCR. Briefly, the single-stranded DNA (˜50-100 ng) was combined with 1× Phusion® buffer, 200 μM dNTPs (each), 0.5 μM each of forward (F) and reverse (R) primers, and one unit Phusion® High-Fidelity DNA Polymerase in a total volume of 50 μL. The primer sequences are as follows: forward (F) 5′-CAA GCA GAA GAC GGC ATA CGA GAT CGG TCT CGG CAT TCC TGC TGA ACC GCT CTT CCG ATC TXX XXC CTT TCT ATT CTC ACT CT-3′ and reverse (R) 5′-AAT GAT ACG GCG ACC ACC GAG ATC TAC ACT CTT TCC CTA CAC GAC GCT CTT CCG ATC TXX XXA CAG TTT CGG CCG A-3′. Of note, XXXX in the primer sequence denotes four-nucleotide-long barcodes used to trace multiple samples in one Illumina sequencing experiment. The temperature cycling protocol for PCR was: 95° C. for 30 s, followed by 25 cycles of 95° C. for 10 s, 60.5° C. for 15 s and 72° C. for 30 s, and a final extension at 72° C. for 5 min before holding at 4° C. The resulting amplicons were pooled (20 ng per sample) together and purified on E-Gel® SizeSelect™ 2% agarose gel (Invitrogen, #G6610-02). Sequencing was performed using the Illumina NextSeq platform (Axil Scientific). Identification of significantly enriched sequences from deep-sequencing data was performed. Heat maps were used to identify sequences isolated from a target selection (eIF4E) that increased significantly in abundance against sequences isolated from the control selection. The copy number of each sequence is normalized through dividing the copy number by the total number of reads in each replicate. Sequences not observed in a specific replicate were assigned a copy number of zero. The ratio of each sequence was calculated through dividing the mean fraction of the particular sequence in the target selection by those in the control screen (e.g., steptavidin). Since the denominator must not be a zero when taking the ratio, sequences with zero copy number found in all three replicates are assigned with 0.3 copy number before taking the normalization. Significance of the ratio was assessed using one-tailed, unequal variance Student t-test. The ratio is considered to be statistically significant if the calculated p-value≤0.05. Only sequences with ratio≥5 and p-value≤0.05 were analysed. Heats maps were plotted showing only the significant sequences and were ranked using their ration values, from highest to lowest. Python scripts were written to perform the described operations.

### Fluorescence Anisotropy Competition Assays and K_{d} Determination

Purified eIF4E and PCNA was titrated against 50 nM carboxyfluorescein (FAM) labelled eIF4E tracer peptide (Ac-KKRYSRDFLLALQK-(FAM)-NH₂) or m⁷GTP^{5-FAM} (Jena Biosciences Cat. No: NU-824-5FM) or PCNA tracer peptide (Ac-SAVLQKKITDYFHPKK-K (5-carboxylysine)-NH2). The K_{d} (dissociation constant) for the titration of eIF4E and PCNA against the tracer peptide was determined by fitting the experimental data to a 1:1 binding model equation:

$$r = {r}_{o} + ( {r}_{b} - {r}_{o} ) \times \frac{( {K}_{d} + {[ L ]}_{t} + {[ P ]}_{t} ) - \sqrt{{{K}_{d} + {[ L ]}_{t} + {[ P ]}_{t} )}^{2} - {{4 [ L ]}_{t} [ P ]}_{t}}}{{2 [ L ]}_{t}}$$

where [P] is the protein concentration, [L] is the labelled peptide concentration, r is the anisotropy measured, r₀ is the anisotropy of the free peptide, r_{b} is the anisotropy of the eIF4E- or PCNA-tracer peptide complex, [L], is the total FAM labelled peptide or m⁷GTP^{FAM} concentration, and [P] is the total eIF4E or PCNA concentration. The K_{d}s determined for the interaction of either the tracer peptide or m⁷GTP^{FAM} with eIF4E were 50.3 nM and 149.0 nM respectively. These were used in subsequent K_{d} determinations in competition experiments to measure binding against the eIF4E cap-binding site and eIF4E:4G interface. The K_{d} determined for the interaction of the tracer peptide or with PCNA was 150 nM. This K_{d} was used in subsequent K_{d} determinations in competition experiments to measure binding against the PCNA protein interaction binding site which is the site where PCNA interacts with more than 200 proteins e.g., p21, p300, CDK2 and others.

To determine K_{d}s for compounds that disrupted the eIF4E:4G interfaces and the PCNA: tracer complex, molecules were titrated against eIF4E or PCNA and the labelled peptide. The molecules titrated against eIF4E and the labelled peptide were at set concentrations of 200 nM and 50 nM, respectively and molecules titrated against PCNA and the labelled peptide were at set concentrations of 625 nM and 50 nM, respectively. With respect to compounds that interacted at the eIF4E cap binding site, titrations were performed with eIF4E and m⁷GTP^{5-FAM} at concentrations of 250 nM and 50 nM. Apparent K_{d} values for both competition assays were determined by fitting the experimental data to the equations shown below:

$$r = {r}_{o} + ( {r}_{b} + {r}_{o} ) \times \frac{2 \sqrt{( {d}^{2} - 3 e )} cos ( θ / 3 ) - 9}{3 {K}_{d 1} + 2 \sqrt{( {d}^{2} - 3 e )} cos ( θ / 3 ) - d}$$

$$d = {K}_{d 1} + {K}_{d 2} + {[ L ]}_{st} + {[ L ]}_{t} - {[ P ]}_{t}$$

$$e = ( {[ L ]}_{t} - {[ P ]}_{t} ) {K}_{d 1} + ( {[ L ]}_{st} - {[ P ]}_{t} ) {K}_{d 2} + {K}_{d 1} {K}_{d 2}$$

$$f = - {K}_{d 1} {{K}_{d 2} [ P ]}_{t}$$

$$θ = ar cos [ \frac{- 2 {d}^{3} + 9 de - 27 f}{2 \sqrt{{( {d}^{2} - 3 e )}^{3}}} ]$$

[L]ₛₜ and [L]ₜ denote labelled ligand and total unlabelled ligand input concentrations, respectively. K_{d2} is the dissociation constant of the interaction between the unlabelled ligand and the protein. In all competitive types of experiments, it is assumed that [P]ₜ>[L]ₛₜ, otherwise considerable amounts of free labelled ligand would always be present and would interfere with measurements. K_{d1} is the apparent K_{d} for the labelled peptide used in the respective experiment. The tracer peptide was dissolved in DMSO at 1 mM and diluted into experimental buffer. Readings were carried out with a Envision Multi-label Reader (PerkinElmer). Experiments were carried out in PBS (2.7 mM KCl, 137 mM NaCl, 10 mM Na₂HPO₄ and 2 mM KH₂PO₄ (pH 7.4)) and 0.1% Tween 20 buffer. All titrations were carried out in triplicate. Curve-fitting was carried out using Prism 4.0 (GraphPad).

### Tryptophan Quenching

Tryptophan fluorescence quenching studies were performed using a Envision Multiplate in a black 96 well plate. Protein samples were excited at a wavelength of 290 nm and tryptophan emission was measured at a wavelength of 355 nm. Sample wells contained eIF4E at a concentration of 10 μM at a set volume of 100 μl with increasing concentrations of the relevant compounds under study. Quenching experiments were performed in PBS buffer (2.7 mM KCl, 137 mM NaCl, 10 mM Na₂HPO₄ and 2 mM KH₂PO₄ (pH 7.4)) with a final DMSO concentration of 1% (v/v).

Construction and Assessment of VH Domains with Rationally Designed Linkers

VH-DiF clones containing different linker regions flanking the eIF4E cap-site interaction motif (EMGFF) were ordered as Ultramer double stranded oligonucleotides (IDT) containing EagI/HIndIII restriction sites. The double stranded VH-Dif encoding cassettes were then cloned into the pET22b bacteria expression vector via ligation at the EagI/HIndIII cloning sites in frame with the c-terminal polyHis affinity purification tag. VH domain constructs (R1-R3) were purified as out lined in the “Bacterial Expression and Purification of VH-Domain constructs” section. Purified VH clones were incubated in a 20:1 excess ratio to purified GST-eIF4E (20 μM) and incubated for 4 hours at room temperature in PBS with 1 mM DTT (see “eIF4E expression and purification for Sortase Labelling”). GST-eIF4E:VH domain complexes were pulled down with 20 μl of GST-beads (Thermo Fisher). Protein samples were analyzed using SDS-PAGE gel and visualized with Coomassie stain.

### Yeast Display PELE Library Construction

The pCT-CON vector was digested using SalI, NheI, and BamHI restriction enzymes (NEB) to ensure complete linearization and absence of full-length insert, thereby preventing transformation of yeast cells with parental plasmid. The PELE library of Dif-VH domains was constructed by three-step overlap extension PCR (OE-PCR). A set of 9 primers; P1_for, P2_rev to P9_rev were dissolved at 100 μM concentration and mixed in an equimolar ratio to prepare three mixed pools containing each primer at a concentration of 10 μM. The three mixed pools were denoted ‘Lib1’, ‘Lib2’, and ‘Lib3’ with each containing a primer specifically encoding a designed PELE library, P9a_for, P9b_for, or P9c for, respectively. 1 μL was taken from each mixed library and 5-fold dilution series prepared to identify the optimal primer concentration for OE-PCR. 0.4 μM of each primer was found to produce optimal yields for OE-PCR for each of the three mixed pools. The full length diF-VH domain product from each library OE-PCR reaction (Lib1, Lib2 and Lib3) was mixed in a 1:1:1 molar ratio (denoted ‘pooled PELE library’). 300 ng of the pooled PELE library and 1 μg of digested pCT-CON vector were combined with 50-100 μL of electrocompetent EBY100 yeast cells and electroporated at 0.54 kV and 25 μF using a GenePulser Xcell (Bio-Rad). Homologous recombination of the linearized vector and pooled PELE insert yielded intact plasmid. Cells were grown in YPD (1% yeast extract, 2% peptone, 2% glucose) for 1 h at 30° C., 250 rpm. The number of total transformants was 5.7×10⁷ cells as determined by serial dilutions plated on SD-CAA plates (0.1 M sodium phosphate, pH 6.0, 182 g/L sorbitol, 6.7 g/L yeast nitrogen base, 5 g/L casamino acids, 20 g/L glucose). The library was propagated by selective growth in SD-CAA, pH 5.3 (0.07 M sodium citrate, pH 5.3, 6.7 g/L yeast nitrogen base, 5 g/L casamino acids, 20 g/L glucose, 0.1 g/L kanamycin, 100 kU/L penicillin, and 0.1 g/L streptomycin) at 30° C., 250 rpm.

### Selection of Yeast Displayed PELE Library

1×10¹⁰ cells were taken from the propagated library culture and pelleted at 2,500 g for 5 min in 50-ml conical tubes and resuspended in SGCAA media to an absorbance of about 0.5-1 at 600 nm to induce expression of the pooled PELE library. Library induction was maintained for 24 hours at 20° C.

1×10 8 induced yeast cells were pelleted at 2500 g for 5 mins and the supernatant aspirated. Cells were then washed in 25 ml PBSM buffer, re-pelleted, supernatant discarded and re-suspended in 10 ml of PBSM. Biotinylated eIF4E was then added to a final concentration of 1 μM. The yeast cell suspension was then incubated at room temperature with gentle rotation on a tube rotator for 60 min, followed by 10 mins on ice.

Dif-VH domain: eIF4E complexes were then isolated using MidiMACS (Mitenyi Biotec) magnetic separation. An LS column (Mitenyi Biotec) was equilibrated with PBSM buffer at 4° C. The yeast cell suspension was then pelleted at 2500 g for 5 min at 4° C., the supernatant aspirated, the yeast cells washed with 50 ml PBSM buffer and then resuspended in 5 ml PBSM buffer. 200 μl of streptavidin microbeads were added to the suspension and incubated on ice with gentle mixing for 10 minutes. Yeast cells were spun down and again washed, before being re-suspended in 50 ml PBSM buffer. Cells were then applied to the LS column in the presence of magnet. Unlabelled cells were washed from the column with 10 ml ice cold PBSM. Cells labelled with streptavidin microbeads were eluted from the column by removal from the magnetic field into a collection tube. Serial dilutions of the sorted cell suspension were plated onto SDCAA plates and incubated at 30° C. to estimate the number of cells captured by MACS. Eluted yeast cells were then propagated with addition of SDCAA (containing 100 units/ml and 100 mg/ml of Penicillin-streptomycin) media to a final volume of 500 ml and incubated overnight at 30° C.

Magnetic sorting was then followed by 2 rounds of FACs enrichment. 1×10⁸ cells were taken from the propagated library culture and pelleted at 2,500 g for 5 min in 50-ml conical tubes and resuspended in SGCAA media to an absorbance of about 0.5-1 at 600 nm to induce expression of the sorted PELE library. Cells were incubated overnight at 20° C. Induced cells were spun down at 14,000 g for 30 s, supernatant aspirated and cells washed with PBSF buffer. Yeast cells were then labelled with 500 nM of sortase labelled biotinylated eIF4E in 1 ml of PBSF buffer and incubated at room temperature for 30 mins. Cells were then pelleted by centrifugation (14,000 g for 30 s at 4° C.), the supernatant aspirated and then washed with 1 ml ice-cold PBSA. Yeast were resuspended in 500 μl PBSF containing Anti-HA Ab Alexa Fluor 488 (Invitrogen, 1:100 fold dilution) and Streptavidin-phycoerythrin (ThermoFisher Scientific, 1:100 fold dilution) and incubated for 30 mins. Cells were then pelleted at 14,000 g for 30 s at 4° C., washed with 1 ml PBSF buffer and resuspended in 2.0 mL PBSF. Cells positive for anti-HA and eIF4E were selected and sorted using an Aria (Becton Dickinson) cytometer. Collected cells were propagated in SDCAA at 30° C. and a second round of FACs selection performed after yeast induction with 1×10⁸ cells as described.

After the final round of FACs selection, serial dilutions of the sorted cell suspension were plated onto SDCAA plates and incubated at 30° C. until the appearance of yeast colonies. X colonies of yeast were individually picked and then propagated in X mls of SCDAA. Plasmid DNA was then isolated using the Zymoprep kit II (following the manufacturer's instructions), cleaned using the Qiagen PCR Purification kit, and transformed into DH5α (Invitrogen) cells. Purified plasmids were then sequenced using BigDye chemistry.

Assessment of Enriched Yeast PELE Library for Specific Binders to the eIF4E Cap-Binding Site

Before the second round of FACs sorting an additional subset of 5×10⁷ cells was induced in 5 ml of SGCAA media. Yeast were then pelleted, washed in 1 mL PBSF resuspended in PBSF to a density of 1×10⁷ cell per ml. 1 ml of yeast suspension was added to three individual tubes. Purified sortase biotinylated eIF4E was then added to each sample at a concentration of 2 μM and samples were incubated at 20° C. for 1 hour. Purified sortase biotinylated eIF4E was then added to each sample at a concentration of 0.2 μM either in combination with 50 μM m⁷GTP (Sigma-Aldrich), 50 μM of purified 4E-BP1^{4ALA} or 50 μM of VH-1C5^{M4}, followed by sample incubation at 20° C. for 1 hour. 4E-BP1^{4ALA} and VH-1C5^{M4} were then purified. The purification of 4E-BP1^{4ALA} mutants is described in the Section “eIF4E and 4EBP1^{4ALA} mutants expression and purification for Sortase Labelling”

For purification of VH-1C5^{M4}, VH-1C5^{M4} sequence were ordered as gene fragments from Integrated DNA Technologies (IDT). Both coding sequences were PCR amplified and cloned directly into the bacterial expression vector pET-22b(+) with an in-frame C-terminal six-histidine tag. VH-M4 was directly PCR amplified from the pETCON2 plasmid used in the yeast alanine scanning experiments, whilst VH-S4 was amplified from the plasmid isolated through the affinity maturation selection. Both sequences were then cloned into pET-22b(+) as described earlier. Using VH-M4 (SEQ ID NO: 100) as a template sequence, the in-fusion mutagenesis kit (Takara) was used to generate the following mutants in the pET-22b(+) backbone (VH-1C5^{D104A/S108R} and VH-1C5^{D104A/F120I}). Each VH domain plasmid was separately transformed into E. coli BL21(DE3) cells and used to inoculate 10 mls of LB broth (containing 100 μg/ml) started culture, which were incubated overnight before being used to seed 1000 ml of fresh LB broth. Bacterial cultures were grown at 37° C. and when they reached a OD600 of 0.6-0.8, the cells were induced with a final concentration 0.5 mM of IPTG and incubated overnight at 25° C. Cells were harvested by centrifugation at 17,000×g for 10 min and pellets were resuspended in lysis buffer (25 mM HEPES pH 7.5, 300 mM NaCl, 20 mM imidazole, 1 mM DTT) and then sonicated for 5 min. Bacterial supernatants were then filtered through 1.2 μm syringe filters. Proteins were purified through a standard two-steps protocol: first, supernatant were loaded onto a 1 ml His Trap column (Cytiva Lifesciences), which was pre-equilibrated then extensively washed with buffer A (25 mM HEPES pH 7.5, 300 mM NaCl, 1 mM DTT) and then eluted with buffer A that also contained 500 mM imidazole; second, the eluted proteins were subjected to gel filtration chromatography on a Superdex 75 column (Cytiva Lifesciences) using PBS buffer containing 1 mM DTT. Protein fractions were analysed by SDS page gel and concentrated. Protein concentration was determined using absorbance at A280 nm.

Cells were then pelleted by centrifugation (14,000 g for 30 s at 4° C.), the supernatant aspirated and then washed with 1 ml ice-cold PBSA. Yeast were resuspended in 500 μl PBSF containing Anti-HA Ab Alexa Fluor 488 (Invitrogen) and Streptavidin-phycoerythrin (ThermoFisher Scientific) and incubated for 30 mins. Cells were then pelleted at 14,000 g for 30 s at 4° C., aspirate supernatant and wash with 1 ml PBSF buffer. Each sample was then analysed by flow cytometry using Aria (Becton Dickinson) cytometer.

### VH Domain Expression and Purification Assessment

VH domains were amplified and cloned into the pET-22b(+) vector (Novagen) using the in-Fusion cloning method (Takara Bio) as described earlier. These VH domains were expressed into E. coli BL21(DE3) cells. Cells were grown at 37° C. and induced protein expression overnight at 25° C. by 0.5 mM Isopropyl-B-D-thiogalactoside (IPTG). For assessment of clones, cells from 20 ml cultures was harvested, and lysed by sonication in lysis buffer (25 mM HEPES pH 7.5, 300 mM NaCl, 20 mM imidazole, 1 mM DTT) supplemented with protease inhibitor cocktail. After centrifugation, the supernatant containing soluble proteins was loaded into Ni-NTA spin column (Qiagen). The column was washed twice using lysis buffer and then eluted with 25 mM HEPES at pH 7.5, 300 mM NaCl, 1 mM DTT and 500 mM imidazole. To assess protein solubility of different VH domains, the eluted proteins were analyzed by SDS-PAGE gel and stained with coomassie blue.

### Bacterial Expression and Purification of VH-Domain Constructs

VH-Dif sequences were ordered as gene fragments from Integrated DNA Technologies (IDT). Coding sequences were PCR amplified and cloned directly into the bacterial expression vector pET-22b(+) with an in-frame C-terminal six-hisitidine tag using the BamHI/XhoI cloning site. Each VH-domain plasmid was separately transformed into E. coli BL21(DE3) cells and used to inoculate 10 ml of LB broth (containing 100 ug/ml) started culture, which were incubated overnight before being used to seed 1000 ml of fresh LB broth. Bacterial cultures were grown at 37° C. and when they reached a OD600 of 0.6-0.8, the cells were induced with a final concentration 0.5 mM IPTG and incubated overnight at 25° C. Cells were harvested by centrifugation at 5,000 rpm for 10 minutes and pellets were resuspended in lysis buffer (25 mM HEPES pH 7.5, 300 mM NaCl, 20 mM imidazole, 1 mM DTT) and then sonicated for 5 minutes. Bacterial supernatants were then filtered through 1.2 μm syringe filters. Proteins were purified through a standard two-step protocol: first, supernatant was loaded onto a pre-equilibrated 1 ml HisTrap column (Cytiva Lifesciences), which was then extensively washed with buffer A (25 mM HEPES pH 7.5, 300 mM NaCl, 1 mM DTT) and then eluted with an imidazole gradient; second, the eluted proteins were subjected to gel filtration chromatography on a Superdex 75 column (Cytiva Lifesciences) using PBS buffer containing 1 mM DTT. Protein fractions were analysed by SDS page gel and concentrated. Protein concentration was determined using absorbance at A280 nm.

### Protein Crystallization

The eIF4E:EE-02 and eIF4E:VH-DiF^{CAP-01} complexes were crystallized by vapour diffusion using the hanging drop method. For crystallization, the eIF4E:EE-02 complex was prepared by direct addition of a 100 mM DMSO stock solution of EE-02 to purified eIF4E recombinant protein (dialysed in 10 mM HEPES 7.6, 100 mM KCL buffer) to generate a final solution of 200 μM eIF4E and 300 μM EE-02 with a residual DMSO concentration of 0.3% (v/v). The sample was then spun down using a tabletop centrifuge at 13,000 g after overnight incubation at 4° C. and the supernatant used for crystallization. The eIF4E:VH-DiF^{CAP-01} complex solution for crystallization was prepared by dialysing both proteins into 10 mM HEPES 7.6, 100 mM KCL and 1 mM DTT buffer and mixing them to give final respective concentrations of 100 μM and 200 μM. Hanging drops were set-up in a pre-greased VDX48 plate (Hampton, USA) with 1 μl of the respective crystallization sample mixed with 1 μl of the mother-well solution. eIF4E:EE-02 crystals grew over a period of one week in 0.2 M Potassium chloride, 20% (w/v) PEG 3350. eIF4E:VH-Dif^{CAP-01} crystals grew over a similar period of time but in 0.1 M TRIS.HCl PH 8.5, 25% (v/v) PEG 550 MME. For X-ray data collection at 100 K, crystals for both sets of crystallization conditions were transferred to an equivalent mother liquor solution containing 25% (v/v) glycerol and then flash frozen in liquid nitrogen.

### Data Collection and Refinement

X-ray diffraction data was collected at the Australian synchrotron (MX1 beamline) using a CCD detector, and integrated and scaled using XDS. The initial phases of the EE02 complexed crystal of eIF4E were solved by molecular replacement with the program PHASER8 using the human eIF4E structure (PDB accession code: 4BEA) as a search model. With respect to the eIF4E:VH-DiF^{CAP-01} co-crystal the VH domain structure (PDB accession code: 5TDP, chain B) was also included in the PHASER molecular replacement search as an independent search model. The starting models were subjected to rigid body refinement and followed by iterative cycles of manual model building in Coot and restrained refinement in Refmac 6.0.9 Models were validated using PROCHECK10 and the MOLPROBITY webserver.11 Final models were analysed using PYMOL (Schrödinger). See table 1 for data collection and refinement statistics. The eIF4E complex structures with EE-02 and eIF4E VH-DIF^{CAP-01} have been deposited in the PDB under the submission codes 7EZW and 7F07, respectively.

TABLE 1
Crystallographic data collection and refinement statistics.
Highest resolution bin data stated in parentheses.

| PDB ID: | 7EZW | | 7F07 | |
|---|---|---|---|---|
| Resolution (Å) | 48.148-2.35, | (2.47-2.35) | 40.996-2.25, | (2.37-2.25) |
| Space Group | P6₃ | | P2₁2₁2₁ | |
| Unit Cell | a = 81.14, b = 81.4, c = 66.11, | | a = 38.52, b = 81.82, c = 122.99, | |
| Dimensions (Å) | α = γ = 90°, β = 120° | | α = β = γ = 90° | |
| Temp (K) | 100 | | 100 | |
| Redundancy | 11.3, | (11.2) | 13.4, | (14.0) |
| Unique Collected | 10511, | (1027) | 19,226, | (2734) |
| Reflections | | | | |
| Completeness (%) | 99.9, | (99.2) | 100, | (100) |
| R Merge (%) | 0.114, | (0.711) | 0.120, | (0.755) |
| I/sigma | 13.8, | (2.6) | 4.7, | (1.0) |
| R factor (%) | 20.53 | | 22.19 | |
| R free (%) | 24.76 | | 27.18 | |
| RMS Bonds (Å) | 0.0023 | | 0.0028 | |
| RMS Angles (°) | 1.143 | | 1.216 | |
| Wilson B-factor (Å²) | 30.5 | | 37.6 | |
| Average Refined B | | | | |
| Factors | | | | |
| Chain A | 35.29, | (eIF4E) | 48.34, | (eIF4E) |
| Chain B | 41.85, | (EE-02) | 56.6, | (VH-DiF^{CAP}-01) |
| Waters | 28.67 | | 46.46, | |
| Number of Water | 59 | | 48 | |
| Molecules | | | | |
| Ramachandran Data | | | | |
| (Rampage). Number of | | | | |
| Residues in (%): | | | | |

| Favoured Region | 96.7 | 97.8 |
|---|---|---|
| Allowed Region | 3.3 | 2.2 |
| Outlier Region | 0 | |

### Isothermal Titration Calorimetry (ITC)

ITC measurements were performed with the Affinity ITC (TA Instruments, USA) at 25° C.

For purified eIF4E, the purified proteins were buffer exchanged into 1×PBS, pH 7.2 with 0.001% Tween-20 using 7K MWCO Zeba spin desalting column (ThermoFisher scientific). 10-30 μM of eIF4E protein was loaded into the sample cell, and 100-300 μM of VH domains were titrated into eIF4E protein, over 15-20 injections of 2.5 μL. All experiments were conducted in duplicate. calorimetric data were analysed with NanoAnalyze software using a one-site binding model.

For purified eIF4A, 10 or 20 μM of eIF4A was loaded in the cell with 100 or 200 μM of the relevant peptide aptamer in the titrating syringe, depending on the binding affinities of compounds. eIF4A was dialysed into Phosphate Buffered Saline (2.7 mM KCL and 137 mM NaCL, pH 7.4) with 0.05% TWEEN20 using SLIDE-A-LYZER (Pierce) cassettes with a MWCO of 3000. Stock VH domain solutions were dialysed side by side using a different cassette in the same buffer as eIF4A. These were then diluted to their working concentration using the dialysis buffer. The titration experiments were performed at 25° C. with a series of 2.5 μl injections (usually 20-30 injections). The spacing between each injection was 300s. The stirring speed during the titration was 75 rpm. Data was analyzed using NanoANALYZE software by fitting to a single-site binding model.

Correction for the enthalpy of ligand dilution was carried out by subtracting a linear fit from the last three data points of the titration, after the interaction had reached saturation.

eIF4A Surface Plasmon Resonance (SPR) Assays

For eIF4A immobilisation in the SPR binding assays, the N-terminally sortase biotinylated eIF4A was immobilized on a streptavidin coated CM5 sensor chip. eIF4A at a concentration of 0.5 M was injected across the chip until approximately 100 RU was immobilised. Streptavidin in the reference channel was blocked with free biotin. Six buffer blanks were first injected to equilibrate the instrument fully. Surface Plasmon resonance experiments were performed on a Biacore T100 machine. Stock protein solutions were serially diluted into running buffer immediately prior to analysis. Running buffer consisted of 10 mM Hepes pH 7.6, 0.15 M NaCl, 1 mM DTT and 0.1% Tween20.

For SPR multi-cycle injection experiments, these injections were performed using a flow rate of 50 μl/min. Peptide aptamers were injected for 60 s and dissociation was monitored for 180 s. Individual proteins were injected across streptavidin coated CM5 chips in threefold dilution series using as appropriate concentration range to determine their respective binding constants. Each independent protein injection sampled one concentration only and was immediately followed by a similar injection of SPR buffer to enable the chip surface to be fully regenerated by dissociation. Responses from the target protein surface were transformed by: (i) subtracting the responses obtained from the reference surface that contained no immobilised protein, and (ii) subtracting the responses of the buffer injections from those of the peptide aptamer injections. The last step is known as double referencing, which corrects the systematic artefacts. K_{d}s were determined using the BiaEvaluation software (Biacore) and calculated from both the response of the eIF4A coated streptavidin CM5 chips at equilibrium and kinetically from the dissociation and association phase data for each of the peptides. Both the equilibrium and kinetic data were fitted to 1:1 binding model. Within each titration, at least two concentration points were duplicated to ensure stability and robustness of the chip surface. Data analysis was performed with Biacore T100 evaluation software (v2.0.4).

### Molecular Dynamics Simulations

The X-ray resolved VH-DIF^{CAP-01}: eIF4E and EE-02: eIF4E complex state structures, along with the free VH-DIF^{CAP-01} domain and EE-02 cyclic peptide derived from the respective complexes were subjected to molecular dynamics simulations in AMBER 1812 using all-atom ff14SB¹³ force field parameters. The N-termini of eIF4E and VH-DIF^{CAP-01} were capped with the ACE functional group, while the C-termini of VH-DIF^{CAP-01} and EE-02 were capped with NME and NHE functional groups respectively. The disulphide bond between residues C2 and C10 in the EE-02 peptide was maintained using the “bond” command in the tleap module of AMBER 18. All the water molecules resolved in the crystal structures were retained for the simulations. The four systems (VH-DIF^{CAP-01}: eIF4E, EE-02:eIF4E, free VH-DIF^{CAP-01} and free EE-02) were placed inside a truncated octahedral box and solvated with TIP3P¹⁴ water by setting a minimum distance of at-least 8 Å between any solute atom and the edge of the box. The electroneutrality of the respective systems was achieved by adding appropriate number of counterions. These systems were then energy minimized using steepest descent and conjugate gradient algorithms, heated to a temperature of 300 K in the NVT ensemble and equilibrated for 500 ps in the NPT ensemble with 1 atm pressure. Production dynamics for VH-DIF^{CAP-01}: eIF4E and EE-02: eIF4E complexes were carried out in triplicates for 200 ns each (cumulative simulation time of 1.2 μs) starting with different initial velocities, while that for free VH-DIF^{CAP-01} domain and EE-02 cyclic peptide was run for 1 us each. All the simulations in the production stage were carried out under NPT conditions. Electrostatic calculations, regulation of temperature and pressure along with the constraining of bonds to hydrogen atoms during the simulations were employed as previously described. The simulation temperature of 300 K was set using langevin dynamics, with a collision frequency of 1.0 ps⁻¹ and the pressure was maintained at 1 atm using weak-coupling with a pressure relaxation time of 1 ps. Periodic boundary conditions in x, y and z directions were appropriately applied. Particle Mesh Ewald method (PME) was used for treating the long range electrostatic interaction. All bonds involving hydrogen atoms were constrained using the SHAKE algorithm. A time step of 2 fs was used and the coordinates were saved every 1 ps. The first 20 ns of the production run were discarded in the analysis of the trajectories to reduce the biasness caused by the similarity in the starting structures of the different systems.

### Analysis of Binding Energy and Water Occupancy During the Molecular Dynamics Simulations

Residue-wise decomposition analysis was carried out using the MM/GBSA (Molecular Mechanics/Generalized Born Surface Area) 16 scheme through the MMPBSA.py script available in AMBER 18. The 3 simulated trajectories of each complex, ie VH-DIF^{CAP-01}: eIF4E and EE-2: eIF4E, were concatenated (200 ns per trajectory) into a composite trajectory of 600 ns and from this, 1000 snapshots at equal intervals, were extracted. Water molecules and counterions were removed from these structures and solvation effects were estimated using the implicit Generalized Born Solvation Model (IGB=2) with salt concentration set to 150 mM. The water occupancy map for VH-DIF^{CAP-01}: eIF4E and free VH-DIF^{CAP-01} simulations was generated using the “grid” command in the CPPTRAJ module of AMBER 18 with cubic grid cells of size 0.5 Å. The water density within each grid cell was computed and plotted using the volume viewer menu as an isosurface representation in the UCSF Chimera visualization software.

### Cell Biology

### Plasmid and Reagents

All plasmids were purchased from Addgene unless indicated otherwise. Mutant VH domains were generated with In-fusion mutagenesis kit (Clontech) and then cloned into either a pCDNA3.1 vector (Thermo Fisher Scientific) harbouring a C-terminal 3× FLAG tag via NheI/BamHI sites or into NanoBit plasmids using the NanoBit PPI starter system (Promega, see manufacturer instruction) to allow mammalian cell expression studies. For lentivirus production, the VH-3×FLAG cassette was sub-cloned into the lentiviral expression vector pCW57 via BamHI/AvrII cloning sites. pcDNA3-rLuc-polIRES-fLuc (bicistronic reporter) ret, eIF4E and eIF4G604-646 NanoBIT and 4EBP1^{4ALA} mutant plasmid generation has been described previously. eIF4E, eIF4G and 4EBP1 mutant cDNAs were synthetized and obtained from IDT (Integrated DNA Technologies). eIF4E and eIF4G604-646 were cloned into NanoBit plasmids using the NanoBit PPI starter system (Promega) using XhoI/EcoRI and NheI/EcoRI cloning sites respectively. 4EBP1 mutants were cloned into pCDNA3.1 vector DNA (Thermo Fisher Scientific) harbouring a C-terminal 3× FLAG tag via NheI/BamHI sites to allow mammalian cell overexpression. For bacterial expression, 4EBP1 mutants were cloned into pGEX6P1 using BamHI/EagI cloning sites. GFP and v-Myc coding sequence residing in a pCMV6 mammalian expression vector were obtained from Origene. The bicistronic luciferase reporter construct pcDNA3-rLuc-polIRES-fLuc was purchased from Addgene.

### Cell Culturing Conditions

All cell lines were cultured in DMEM cell media supplemented with 10% foetal calf serum (FBS) and penicillin/streptomycin. Mammalian cells were maintained in a 37° C. humidified incubator with 5% CO₂ atmosphere.

Immunoprecipitation and m⁷GTP Pull Down Experiments

Twenty-four hours prior to transfection or drugging, cells were seeded at a cell density of 1000.000 (HEK293) cells per well of a six-well plate (ThermoFisher Scientific). Transfections were performed using Lipofectamine 3000 (ThermoFisher Scientific) with either 2 μg or the indicated amount of plasmid vectors per well according to the manufacturer's instructions. After a 48-hour incubation period, the cell media was then removed and the cells washed with PBS saline. Cells were directly lysed in the wells with 300 μl of lysis buffer containing 20 mM Hepes pH 7.4, 100 mM NaCl, 5 mM MgCl₂, 0.5% NP-40, 1 mM DTT, with protease (Roche) and phosphatase (Sigma-Aldrich) inhibitor cocktail sets added as outlined by the manufacturer's protocols. Cellular debris was removed by centrifugation, and the protein concentration was then determined using the BCA system (Pierce). m⁷GTP pulldown and FLAG immunoprecipitation experiments were performed with 200 μg of cell lysate, which was either incubated with 20 μl of m⁷GTP (Jena Bioscience) or anti-FLAG M2 antibody (Roche) immobilised agarose beads for 2-4 hours at 4° C. on a rotator. Beads were then washed four times with lysis buffer containing no protease or phosphatase inhibitors. This was then followed by the addition of Laemlee buffer (2×) and the beads boiled for 5 min at 95° C. Samples were centrifuged and the supernatant removed for western blot analysis.

### NanoBit Complementation Assay

For NanoBIT (PROMEGA) system development and validation, opaque 96-well plates were seeded with 30,000 HEK293 cells per well in DMEM and 10% FCS and transfected with 30 ng total DNA of the two NanoBit plasmid vectors and 100 μg of the indicated plasmid per well using FUGENE6 (Roche). 48 hours after transfection, the medium was replaced with 100 μl of Opti-MEM cell media containing 0% FCS with no added red phenol (Thermo Fisher Scientific). To screen indicated compounds using the NanoBIT system in live and permeabilized cells, 6-well plate was seeded with 1300,000 cells per well in DMEM and 10% FCS and transfected with 2 ug total DNA of the two NanoBit plasmid per well. After 24 hrs, transfected cells were trypsinised and re-suspended in Opti-MEM media with 10% FCS. Cells were then spun down at 1000 rpm for 5 minutes at room temperature. Supernatant was then discarded and cells re-suspended to a density of to 220,000 cells per ml in Opti-MEM I reduced serum containing 10% FCS with no added red phenol. 100 μl of cells were added to the wells of a white opaque 96-well plate and incubated for 24 hours at 37° C., 5% CO2. For assessment of permeabilised or live cells, cell medium was replaced with 90 ul of serum free Opti-MEM media that either contained or did not contain 50 μg/ml digitonin, respectively. Live or permeabilized cells were then treated with either 10 μl of a 10% v/v DMSO vehicle control in FPLC grade water or a suitable 2-fold dilution series of the compound under study in a 10-fold higher stock concentration (containing 10% DMSO and FPLC grade water solution). 96 wells were then incubated for 3 hrs at 37° C., 5% CO2. Luminescence activity was assayed as described elsewhere¹⁸ using an Envision Multi-Plate reader.

### Cap-Dependent Translation Assay

Opaque 96-well plates were seeded with 30,000 HEK293 cells per well in DMEM and 10% FCS. Transfections were performed using FUGENE6 (Roche) with 30 ng of the bicistronic reporter (pcDNA3-rLuc-polIRES-fLuc) plasmid and 75 or 150 ng of the indicated plasmid. 48 hours after transfection, Renilla and firefly luminescence activity was determined using the Dual Glo Luciferase Assay System (PROMEGA). Luminescence readings were performed using an Envision Multi-plate reader (PerkinElmer).

### Protein Expression Analysis

Transfected HEK293 cells (prepared as described in the NanoBit and Cap-dependent translation Experiments sections) were seeded with 30,000 cells per well in 96-well plates. After an incubation period indicated in the relevant figure, cells were washed with PBS and directly lysed in the wells of the plate with 50 μl of cell lysis buffer (20 mM Hepes pH 7.4, 100 mM NaCl, 5 mM MgCl2, 0.5% NP-40, 1 mM dithiothreitol) containing the protease (Roche) and phosphatase (Sigma-Aldrich) inhibitor cocktail sets (added as outlined by the manufacturer's protocols). Cellular debris was separated by centrifugation. Samples were analysed by western blot without further quantification.

### Generation of Stably Transfected A375 Cell Lines Expressing PCNA Interacting Peptide Aptamers

Confluent HEK293FT cells were used to generate lentivirus for infection of target cells. Packaging cells were transfected using calcium phosphate transfection as described below. 6 μg of pCW57 plasmid (Addgene, USA) harbouring either 4EBP1^{4ALA} or VH-S4 or no insert were co-transfected into HEK293T cells with plasmids encoding pLVSVG (viral envelope), pLP1 (gag-pol) and pLP2 (rev), in a ratio of 2:1:2:2 to generate viral particles. The conditioned medium harbouring viral particles from the transfected HEK293T cells was filtered 48 hours following co-transfection and viral particles were concentrated by ultracentrifugation. A375 cells were seeded in 12-well plates and infected with viral particles over a 12 hour period prior to cell media replacement with fresh medium 72 hours post infection. A375 cells were supplemented with 800 μg/ml of geneticin and selections for stably transfected cells were carried out for 2 weeks, replacing the antibiotic-containing media every 3 days. Polyclonal geneticin-resistant pools of cells were then obtained. These were then incubated with 1 μg/ml of doxycycline for 24 hours, where upon GFP positive single clones were isolated by FACs into 96-well plates. Monoclonal stable cell lines were verified using western blot and then expanded for subsequent analysis.

### Western Blot Analysis

Samples were resolved on midi or mini Tris-Glycine 4-20% gradient gels (Bio-Rad) according to the manufacturer's protocol. Western transfer was performed with an Immuno-blot PVDF or nitrocellulose membrane (Bio-Rad) using a Trans-Blot Turbo system (Bio-Rad). Western blots were then performed. Antibodies against peIF4E^{S209},4EBP1, cyclin D1 and FLAG were purchased from Abcam or Sigma, respectively. All other antibodies used were purchased from Cell Signalling Technology. B-actin levels were measured to ensure equal loading.

### Cell Proliferation Assay

A375 cell lines were plated in 96-well clear bottom plates at a density of 4000 cells per well in 200 μl DMEM and 10% FCS medium. After 24 hours, cell media was replaced with 200 μl of medium containing doxycycline at 1 μg/ml. Cell confluence and cell growth was then measured continuously over 7 days using an IncuCyte FLR instrument (EssenBioscience).

### Results

### Example 1

Discovery of a Novel eIF4E Cap-Binding Cyclic Peptide Binding Motif

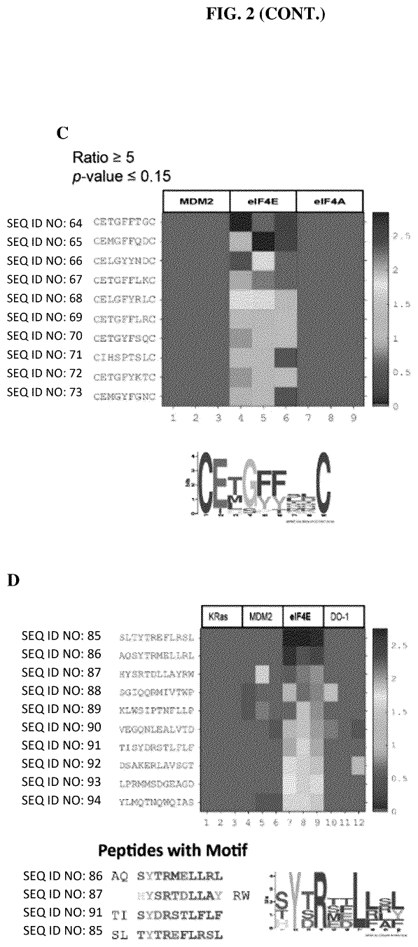

Two M13 phage peptide libraries (New England Biolabs) either consisting of 7 randomised amino acids constrained by a disulphide bond formed between 2 cysteine residues (ACXXXXXXXC, X=any amino) or a hypervariable linear 12mer sequence were panned against biotinylated eIF4E. Parallel selections were performed concurrently against several negative control biotinylated proteins also. The panning culminated in single round to avoid selection of fast-growing phage clones. Recovered phage populations were then subjected to NGS (Next Generation Sequencing, Illumina NextSEQ technology) sequencing, whereupon differential enrichment analysis was performed to identify peptide sequences that specifically bound eIF4E over the control proteins (FIG. 2B). The 12mer library selection identified peptides with the interaction motif (YXRXXL[L/R/F])), which is highly similar to the well-known eIF4E binding motif (YXXXXLϕ, ϕ is any hydrophobic amino acid). In contrast, the motif enriched in the disulphide constrained peptide selection isolated a previously unknown putative eIF4E interaction motif (CE[M/L/T]G[F/Y]XXC) (FIGS. 2C and 2D).

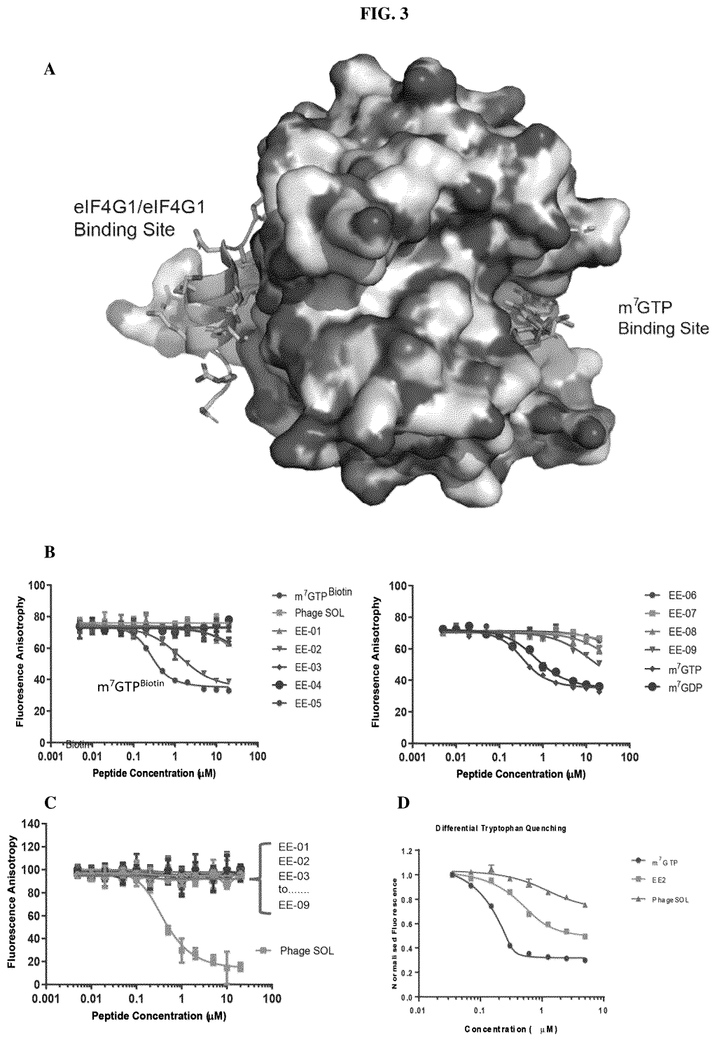

In the absence of any sequence similarity with described eIF4E interacting proteins (eIF4G1 and the 4E-BP family) and the eIF4E interaction motif (enriched in the 12mer selection), competitive fluorescence anisotropy experiments were performed to delineate the binding sites of the EE-01 to EE-09 peptides on eIF4E utilising either a FAM labelled m⁷GTP (m⁷GTP^{FAM}) or a FAM labelled eIF4G/4E-BP1 site interacting peptide (eIF4G^{FAM}) (FIGS. 3A, 3B and 3C). None of the disulphide constrained peptides were observed to displace eIF4G^{FAM} from eIF4E, however several cyclic peptides did compete for binding at the cap-site with m⁷GTP^{FAM}. EE-02 was determined to interact with eIF4E with a K_{d} of 406.2±3.6 nM, 12-fold more strongly than the next best performing cyclic peptide (EE-09, K_{d}=4,860±633.4 nM). Alanine scanning mutagenesis experiments confirmed that the residues conserved in disulphide the constrained peptide motif (C²E³[M/L/T]⁴G⁵[F/Y]⁶[F/Y]⁷X⁸X⁹C, FIG. 2A) were all necessary for binding (Table 2). Mutation of Q8A in the EE-02 sequence (SEQ ID NO: 79) had no effect on binding to eIF4E, whilst the replacement of D9A (SEQ ID NO: 80) resulted in a small attenuation of the K_{d} (Table 2). Additionally, the dependence of EE-02 binding with eIF4E upon the disulphide constraint was demonstrated to be critical under reducing conditions.

Tryptophan quenching experiments were performed to compare the binding mechanisms of EE-02 with m⁷GTP and PHAGESOL (a phage modified eIF4E interacting peptide that binds at the eIF4G binding site) against eIF4E. It is well established that the binding of m⁷GTP to eIF4E results in significant tryptophan fluorescent quenching of W56 and W102 (FIG. 3A), both involved in recognising the m⁷G moiety of m⁷GTP (FIG. 3D), whilst peptides that interact with eIF4E at the eIF4G site also result in quenching of W73 (FIGS. 3A and 3D). However, the reduction in total eIF4E fluorescence caused by peptide binding is significantly less compared to m⁷GTP binding at the cap-site (FIG. 3D). In contrast, the quenching of intrinsic tryptophan fluorescence by EE-02 produces a significantly different profile to both m⁷GTP and PHAGESOL (FIG. 3D), indicating that EE-02 binds via a mechanism substantially different to both.

TABLE 2
Binding assessment of disulphide constrained peptides isolated using M13 phage
display against eIF4AE (EE-02 to EE-09) and alanine scanning mutants exploring
the interaction profile of EE-02 under non-reducing conditions. The binding sites of
the peptides EE-01 to EE-09 were mapped onto eIF4AE using two competitive based
fluorescence anisotropy assays, one of which used a FAM labelled m⁷GTP (m⁷GTP^{FAM}) to
monitor for binding at the cap-binding site, whilst the other assay used a FAM labelled
canonical site interacting peptide (eIF4AG^{FAM}) to measure binding at the eIF4AG interaction
site. Dissociation constants were determined using a 1:1 binding model and are described
in the materials and methods. m⁷GTP, m⁷GDP and m⁷GTP^{BIOTIN} were used as positive
controls for the m⁷GTP^{FAM} competition assay, whilst PHAGESOL was used as a positive
control for the eIF4AG^{FAM} competition assay. EE-02 alanine mutant derivatives were only
assessed for binding in the m⁷GTP^{FAM} competition assay ND = Not determined. K_{d}s >
20,000 were denoted as non-binders. Experiments were performed in triplicate.

| | | Competitive Anisotropy (k_{d}, nM) | |
|---|---|---|---|
| | Sequence | m⁷GTP^{FAM} | eIF4AG^{FAM} |
| m⁷GDP | — | 200.7 ± 25.2 | No Binding |
| m⁷GTP | — | 66.6 ± 7.2 | No Binding |
| m⁷GTP^{BIOTIN} | — | 34.7 ± 4.0 | No Binding |
| EE-01 (SEQ ID | NH₂-ACETGFFTGCG- | 15,030.0 ± 3,078.0 | No Binding |
| NO: 64) | NH₂ | | |
| EE-02 (SEQ ID | NH₂-ACEMGFFQDCG- | 406.2 ± 3.6 | No Binding |
| NO: 65) | NH₂ | | |
| EE-03 (SEQ ID | NH₂-ACELGYYNDCG- | No Binding | No Binding |
| NO: 66) | NH₂ | | |
| EE-04 (SEQ ID | NH₂-ACETGFFLKCG- | No Binding | No Binding |
| NO: 67) | NH₂ | | |
| EE-05 (SEQ ID | NH₂-ACELGFYRLCG- | No Binding | No Binding |
| NO: 68) | NH₂ | | |
| EE-06 (SEQ ID | NH₂-ACETGFFLRCG- | No Binding | No Binding |
| NO: 69) | NH₂ | | |
| EE-07 (SEQ ID | NH₂-ACETGYFSQCG- | No Binding | No Binding |
| NO: 70) | NH₂ | | |
| EE-08 (SEQ ID | NH₂-ACETGFYKTCG- | 16,710 ± 3,027.0 | No Binding |
| NO: 72) | NH₂ | | |
| EE-09 (SEQ ID | NH₂-ACEMGYFGNCG- | 4,860 ± 633.4 | No Binding |
| NO: 73) | NH₂ | | |
| PHAGESOL | Ac-KKRYSR*QLL*-NH₂ | No Binding | 34.05 ± 6.42 |
| EE-02^{E3A} (SEQ | NH₂-ACAMGFFQDCG- | No Binding | ND |
| ID NO: 74) | NH₂ | | |
| EE-02^{M4A} (SEQ | NH₂-ACEAGFFQDCG- | No Binding | ND |
| ID NO: 75) | NH₂ | | |
| EE-02^{G5A} (SEQ | NH₂-ACEMAFFQDCG- | No Binding | ND |
| ID NO: 76) | NH₂ | | |
| EE-02^{F6A} (SEQ | NH₂-ACEMGAFQDCG- | No Binding | ND |
| ID NO: 77) | NH₂ | | |
| EE-02^{F7A} (SEQ | NH₂-ACEMGFAQDCG- | No Binding | ND |
| ID NO: 78) | NH₂ | | |
| EE-02^{98A} (SEQ | NH₂-ACEMGFFADCG- | 384.9 ± 62.1 | ND |
| ID NO: 79) | NH₂ | | |
| EE-02^{D9A} (SEQ | NH₂-ACEMGFFQACG- | 1,682 ± 537.4 | ND |
| ID NO: 80) | NH₂ | | |
| EE-02^{N-Del} (SEQ | NH₂-CEMGFFQDCG- | 66.5 ± 19.5 | ND |
| ID NO: 81) | NH₂ | | |
| EE-02^{C-Del} (SEQ | NH₂-ACEMGFFQDC- | 684.8 ± 54.8 | ND |
| ID NO: 82) | NH₂ | | |
| EE-02^{NC-Del} (SEQ | NH₂-CEMGFFQDC- | 525.9 ± 128.8 | ND |
| ID NO: 83) | NH₂ | | |

the Constrained Macrocyclic Peptide EE-02 Interacts with eIF4E Cap-Binding Site Via a Unique Binding Pose

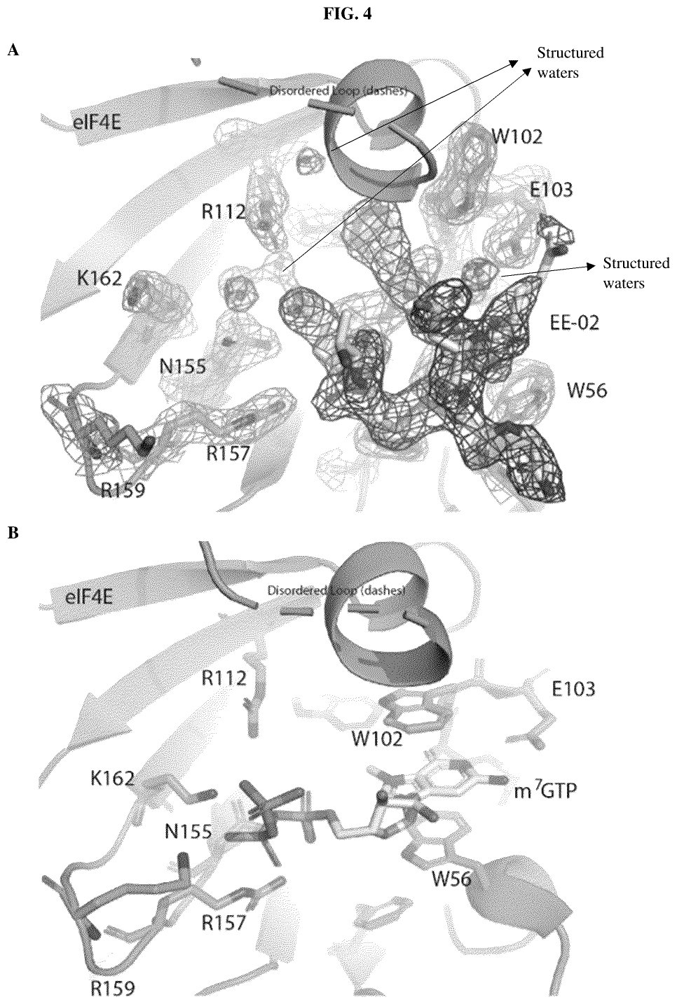

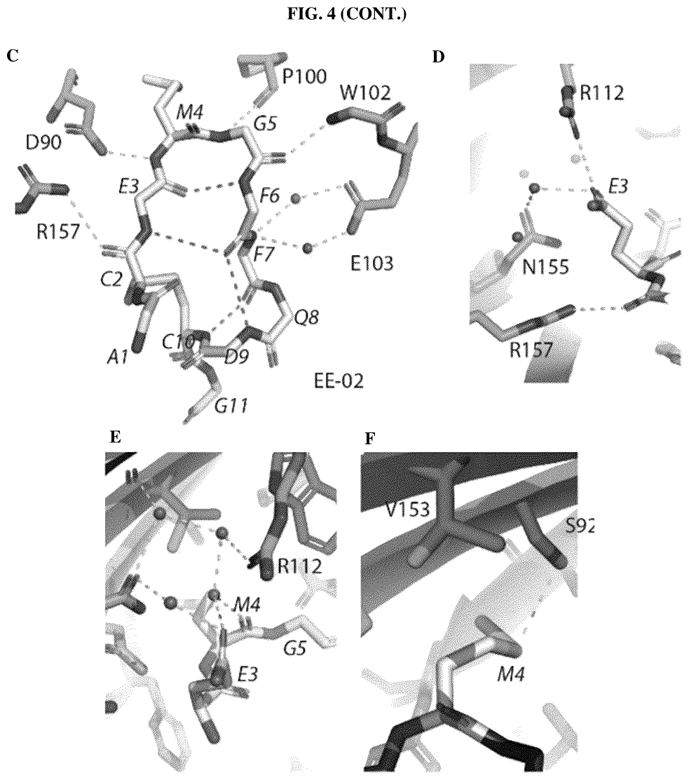

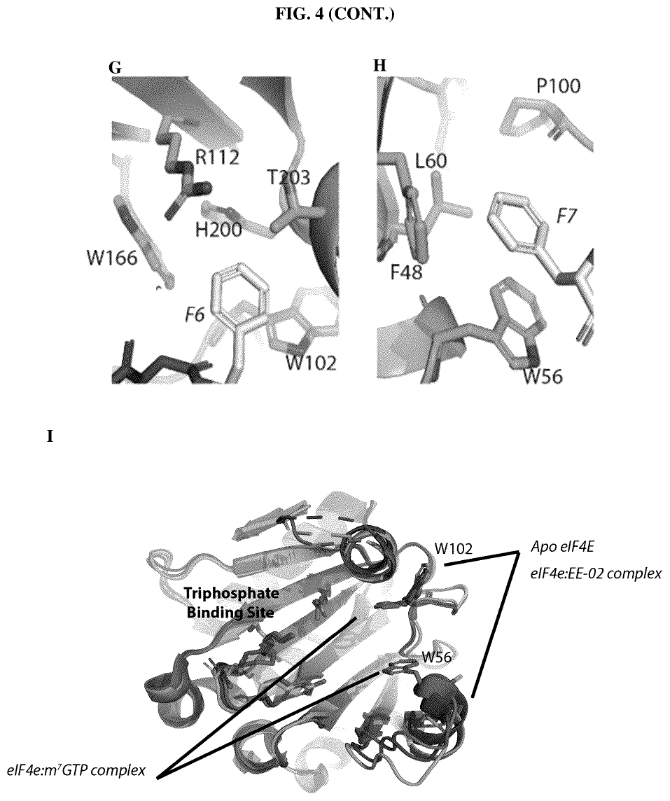

The structure of the EE-02 complex was solved using X-ray crystallography confirming EE-02 bound to eIF4E at the cap-binding site (FIG. 4A), but more interestingly revealed that the site had undergone substantial conformational changes compared to the structure of cap-bound eIF4E (FIG. 4B). These changes were primarily localised to the W56 containing loop (48-60) with smaller sidechain structural changes occurring elsewhere around the pocket. The net effect of these changes was that the side chains of W56 and W102 that play critical roles in recognising the m⁷G moiety of the cap-analogue no longer reside within the cap-binding site and make contrasting interactions with EE-02 compared to m⁷GTP. The differences between the EE-02: eIF4E and m⁷GTP: eIF4E complex structures also explain the substantial differences observed in their tryptophan quenching profiles (FIG. 3D).

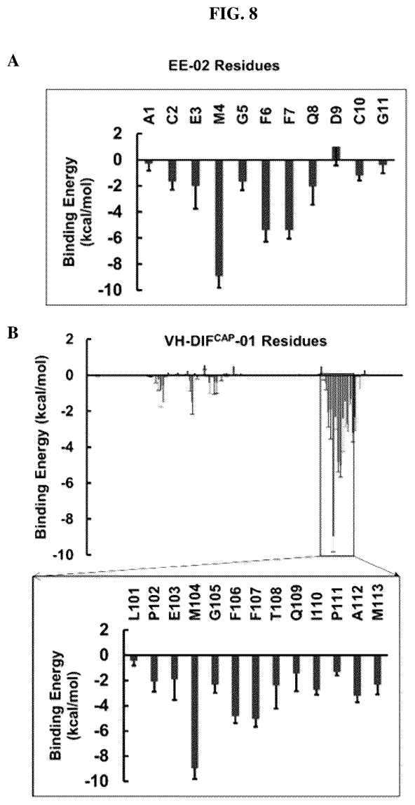

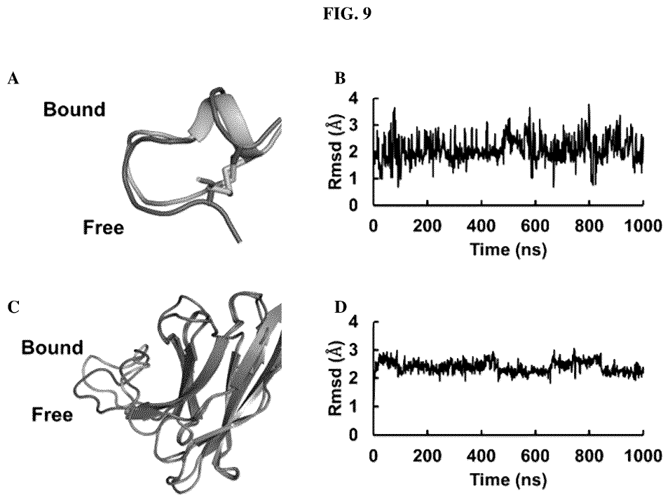

The EE-02 peptide forms a β-hairpin turn-like structure in the binding pocket that allows the side chains of the constrained peptide motif to efficiently interact with eIF4E (FIG. 4C). The glycine at position 5 due to its steric permissiveness enables optimal formation of the B-turn type structure, and in turn a stabilising intramolecular h-bond between the backbone carbonyl of E3 and backbone amide of F6. The E3 of the selected motif (E³MGFF⁷) forms direct electrostatic interactions with R112 of eIF4E (FIG. 4D), whilst M4 forms hydrophobic contacts with the back of the binding pocket and a specific hydrogen bond with S92 via its sulphur atom (FIG. 4E). Residue F6 forms a range of hydrophobic interactions (3.6 Å-4.2 Å) that include T203 A204, H200, W166 and W102 of eIF4E (FIG. 4F). In contrast, F7 forms stacking interactions with W56 and edge on face interactions with F48 (FIG. 4G). Additional main-chain interactions are also formed by EE-02 that contribute to the energetics of binding with eIF4E: the backbone carbonyl of C2 interacts with the R157 sidechain (FIG. 4C), the carbonyl and amide back bones of G5 and F7, respectively, form water mediated interactions with the carboxylic group of E103 (FIG. 4C), and the backbone carbonyl of M4 interacts with a structure water network that involves h-bonds with N155 and R112 (FIG. 4G). Binding energy decomposition analysis from MD simulations of the eIF4E:EE-02 complex structure demonstrates that M4, F6 and F7 contribute a significant proportion of the binding energy (FIG. 8A). The part of the cap-binding that recognizes the triphosphate tail (R157, K159 and K162) of m⁷GTP is not involved in binding EE-02, and undergoes negligible structural changes. Interestingly, the conformation of the eIF4E cap-binding site when bound to EE-02 is very similar to its unbound configuration, where the W102 side chain and the W56 loop also rotate and swing out of the cap-binding site (FIG. 4I). MD simulations of the unbound EE-02 were performed, indicating that its structure does not vary dramatically from the bound form and overall retains a similar fold to that observed in the crystal form (FIGS. 9A and 9B).

Design and Development of a Novel Miniprotein that Interacts with the Cap-Binding Site of eIF4E

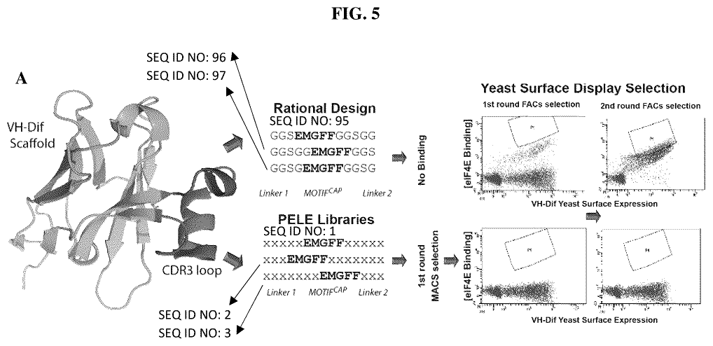

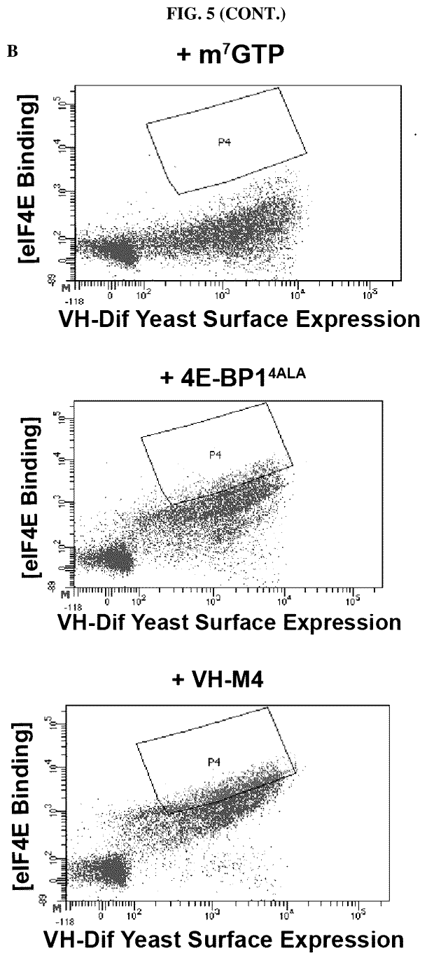

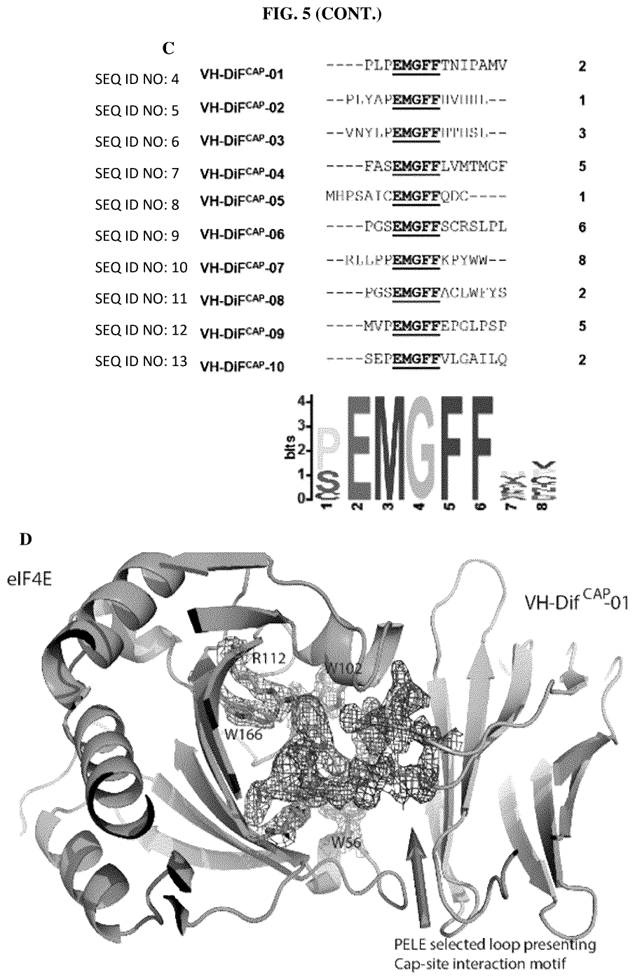

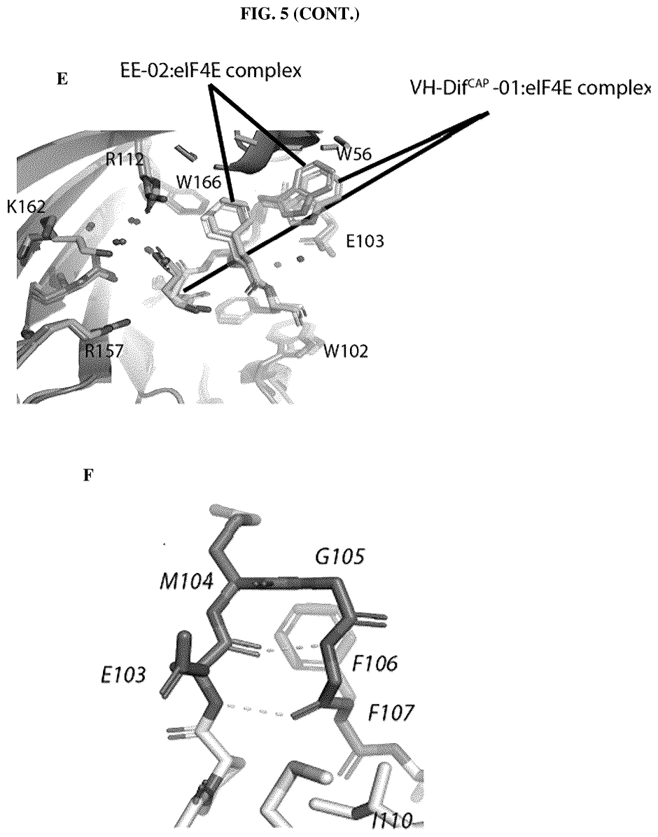

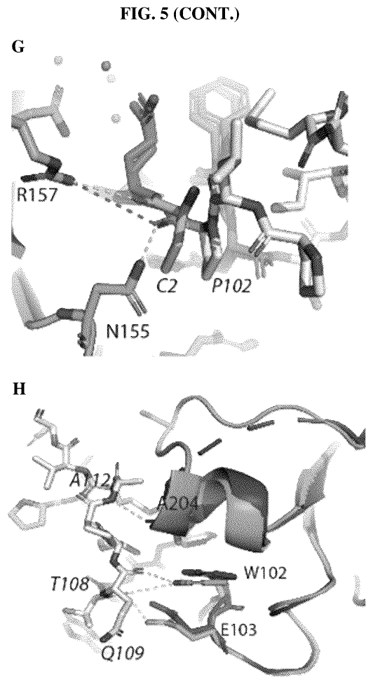

The EE-02 binding epitope was grafted into the CDR3 loop region of an engineered monomeric VH-domain, termed DiF-VH. The DiF-VH scaffold has several attractive features: 1) relatively large peptide insertions can be made into the CDR3 loop region and 2) the protein scaffold is amenable to expression in E. Coli and mammalian cells. Additionally, the points where the CDR3 loop initiates and terminates itself in the VH domain are spatially close together, suggesting that the protein scaffold can act as structural constraint that mimics the function of the disulphide bond in the cyclic peptide (FIG. 5A).

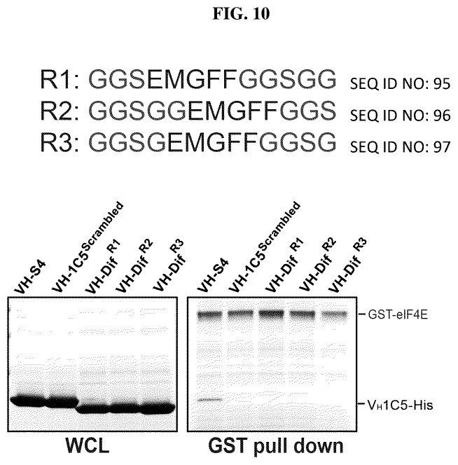

Initial designs where the cyclic eIF4E interaction motif (EMGFF) was introduced at different positions within a 15mer loop in the CDR3 region of the VH-DiF scaffold were tested and none exhibited any binding to eIF4E (FIGS. 5A and 10). It was then hypothesized that the use of poly-glycine linkers for epitope presentation was too permissive and did not restrain the motif sufficiently in the correct structural conformation to be able to interact with eIF4E. To overcome this issue an alternative approach termed Peptide Epitope Linker Evolution (PELE) was adopted, wherein the linker regions were randomized, and a yeast surface display (YSD) library generated to select for linkers that optimally displayed the eIF4E interaction motif (FIG. 5A). To confirm that the PELE selection was successful, input samples of the enriched yeast library from the final selection round were used in competition experiments with either m⁷GTP, 4E-BP1 or VH-M4 (a VH domain that binds at the eIF4G interaction site of eIF4E; (SEQ ID NO: 100)) against biotinylated eIF4E, which confirmed that the binding of the selectants to eIF4E were specific to the cap-binding site (FIG. 5B). 34 yeast clones from the final round of YSD were sequenced, which yielded 10 unique VH-DiF peptide aptamers (termed VH-DiF^{CAP}) (FIG. 5C). Sequence analysis revealed that eIF4E interactors were isolated from each of the PELE libraries used in the selection and that proline was preferentially selected for at the amino acid position preceding the interaction motif (FIG. 5C). The VH-DiF^{CAP} peptide aptamers were then tested for bacterial expression, where upon those with good expression levels (VH-DiF^{CAP-01} (SEQ ID NO: 4), VH-DiF^{CAP-02}, VH-DiF^{CAP-06} (SEQ ID NO: 9), VH-DIF^{CAP-09} (SEQ ID NO: 12)) were purified and screened for binding against eIF4E using the m⁷GTP^{FAM} competition assay (FIG. 11, Table 3). The VH-DiF^{CAP} peptide aptamers that demonstrated binding in the competition assay including the constrained peptide EE-02, were then re-measured using ITC in direct binding titrations, which identified VH-DiF^{CAP-01} as the most potent eIF4E binder with a K_{d} of 35.3±17.0 nM (Table 3). A K_{d} approximately equivalent to that determined for the constrained peptide EE-02.

TABLE 3
Dissociation constants were determined using both the m⁷GTP^{FAM} competitive
anisotropy assay and isothermal calorimetry (ITC) for selected constrained
peptides and peptide aptamers. ITC was also used to determine the following:
enthalpy of binding (ΔH), entropy of (ΔS) bindingand the stoichiometry of
the interaction (N, number of binding sites). Experiments were carried out
at 293 K. Experiments were performed in triplicate. ND = Not determined.

| | | Competitive | | | | |
|---|---|---|---|---|---|---|
| | | Anisotropy | ITC | | | |
| | | (m⁷GTP^{FAM}, | | ΔH | ΔS | |
| | Sequence | k_{d}, nM) | K_{d} (nM) | (KJ/Mol) | (J/mol-K) | N |
| EE-02 | NH₂-ACEMGFFQDCG-NH₂ | 406.2 ± | 63.9 ± | 25.5 ± | −16.4± | 1.3 ± |
| (SEQ | | 3.6 | 18.8 | 2.1 | 3.1 | 0.1 |
| ID NO: | | | | | | |
| 75) | | | | | | |
| EE-02^{M4A} | NH₂-ACEAGFFQDCG-NH₂ | No Binding | ND | ND | ND | ND |
| (SEQ | | | | | | |
| ID NO: | | | | | | |
| 65) | | | | | | |
| VH- | MHPSAICEMGFFQDC---- | No Binding | 2152.5 ± | −7.5 ± | 83.4 ± | 0.99 ± |
| DiF^{CAP}_05 | | | 184.5 | 1.4 | 5.4 | 0.1 |
| (SEQ | | | | | | |
| ID NO: | | | | | | |
| 8) | | | | | | |
| VH- | ----MVPEMGFFEPGLPSP | 3,630 ± | 421.9 ± | −47.1 ± | −35.6 ± | 1.0± |
| DiF^{CAP}_09 | | 300 | 82.6 | 2.6 | 10.0 | 0.1 |
| (SEQ | | | | | | |
| ID NO: | | | | | | |
| 12) | | | | | | |
| VH- | ---PLPEMGFFTNIPAMV | 501.0 ± | 36.3 ± | −31.0 ± | 39.5 ± | 1.0 ± |
| DiF^{CAP}_01 | | 58.0 | 17.0 | 1.7 | 10.0 | 0.1 |
| (SEQ | | | | | | |
| ID NO: | | | | | | |
| 4) | | | | | | |
| VH- | --PLYAPEMGFFHVHHL-- | No Binding | ND | ND | ND | ND |
| DiF^{CAP}_02 | | | | | | |
| (SEQ | | | | | | |
| ID NO: | | | | | | |
| 5) | | | | | | |
| VH- | ----PLPEAGFFTNIPAMV | No Binding | ND | ND | ND | ND |
| DiF^{CAP}_ | | | | | | |
| 01^{Cntrl} | | | | | | |

VH-DIF^{CAP-01} Recapitulates the Interactions of EE-02 with the Cap-Binding Site and Forms Additional Interactions

Crystallization of the VH-DIF^{CAP-01}:eIF4E complex (FIG. 5D) confirmed the residues of the cyclic peptide interaction motif located in the CDR3 loop recapitulated the critical interactions observed between EE-02 and eIF4E (FIGS. 5E and 5F). Additionally, binding energy binding decomposition analysis from MD simulations of the VH-DIF^{CAP-01}:eIF4E complex further confirmed the similarity in the energetics of binding between EE-02 and the evolved CDR3 loop with M104, F106 and F107 again making the largest contributions to the binding energy (FIG. 8B). The only significant deviations in the interaction of EE-02 and VH-DIF^{CAP}-01 with eIF4E were: 1) a small conformational difference in the E103 sidechain and the position of its Ca backbone atom (FIG. 5G), 2) the loss of water-mediated interactions with E103 of eIF4E (FIG. 5G) and 3) a deviation in the packing of the W102 residue against F107 of VH-DIF^{CAP-01} (FIG. 5G). The re-orientation of the E103 residue is principally caused by the P¹⁰⁰LP¹⁰² linker region of VH-DIF^{CAP-01} approaching the cap-binding site at a significantly different angle compared to the orientation of the EE-02 peptide backbone induced by the disulphide bond constraint (FIG. 5F). Interestingly, the changes observed in relation to W102 and the loss of the water-mediated interactions with E103 are primarily driven by the interactions of the evolved linker (T¹⁰⁸NIPAMV¹¹⁴) with eIF4E.

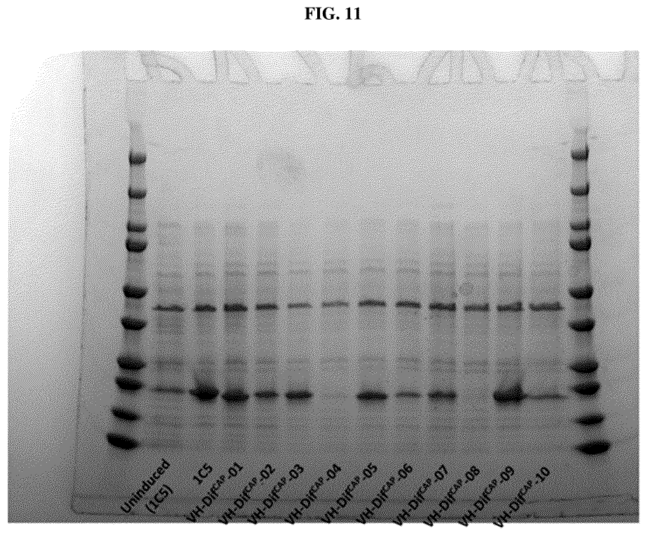

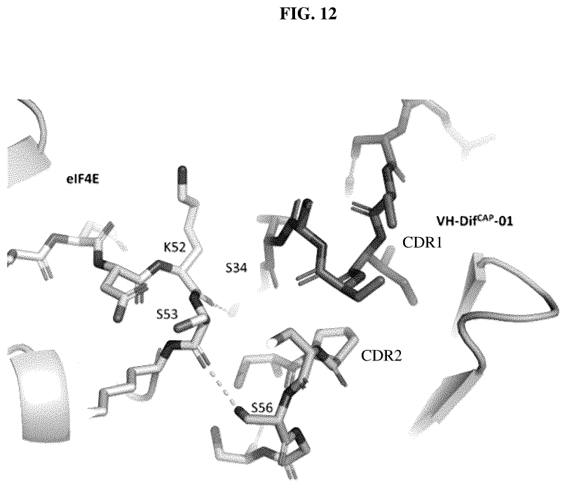

The evolved linker regions of VH-DIF^{CAP-01}, in addition to presenting the EE-02 interaction epitope optimally to interact with eIF4E, also forms multiple additional interactions with eIF4E (FIGS. 5G, 5H and 5I). This contrasts sharply with the EE-02 cyclic peptide where only the Cys2 carbonyl group forms a hydrogen bond directly with R157 outside the residues critical for interacting with eIF4E. This hydrogen bond does not occur in the eIF4E:VH-DiF^{CAP-01}: eIF4E complex structure where but is mimicked by a hydrogen bond between the carbonyl of the P102 in the P¹⁰⁰LP¹⁰² linker region with the side chain of N155 of eIF4e (FIG. 5G). The remainder of the N-terminal PLP linker forms no other interactions with eIF4E. However, the linker section (T108NIPAMV¹¹⁴) that occurs at the C-terminal of the interaction motif in VH-DIF^{CAP-01}, forms most of the interactions made between eIF4E and the evolved linker regions. Residues T108 and Q109 form 2 hydrogen bonds with the indole group of W102 of eIF4E (3.7 Å and 3.1 Å, respectively). An additional hydrogen bond between the linker region and eIF4E is formed between the amide and carbonyl groups of A112 and eIF4E's A204, respectively (FIG. 5H). The remaining residues in the linker (110-114) apart from V114 make a multitude of hydrophobic contacts with residues T203, A204, T206 and G208 in eIF4E, resulting in stabilization of the α-helical secondary structure of this region of the protein (FIG. 5I). V114 in contrast is involved in interactions with the invariant part of the VH-DiF scaffold. VH-DIF^{CAP-01} also interacts weakly at a second site with eIF4E that is constituted from the CDR1 and CDR2 loops of the VH domain, where the residues S34 and S56 both form hydrogen bonds via their sidechains with the carbonyls of K52 and S53, respectively (FIG. 12).

### the Conformation of the CDR3 Interaction Loop is Stabilized by an Intricate Set of Interactions

The strategy of evolving the peptide sequence regions at either side of the cap binding interaction motif to enable optimal presentation of the epitope must also inherently accommodate the presence of residues that occur on the VH domain itself. This is difficult to achieve using the rational design approaches. Several significant features in the linker region of the CDR3 loop that stabilized presentation of the CDR3 loop through packing interactions with the scaffold were noted: 1) the Ile110 sidechain in the linker region forms a hydrophobic core in the CDR3 loop structure that makes multiple hydrophobic contacts with other residues in the linker and the interaction motif, 2) the CDR3 loop when bound to eIF4E forms a folded structure against the R51 and D36 residue of VH-DIF^{CAP-01} which form a salt bridge between each other, 3) and that the linker residue T108 also packs directly against W48, which is found in the scaffold (FIG. 5J). The V114 residue of the evolved linker region is also involved in additional contacts with the VH domain that stabilize the fold of the CDR3 loop (FIG. 5J).

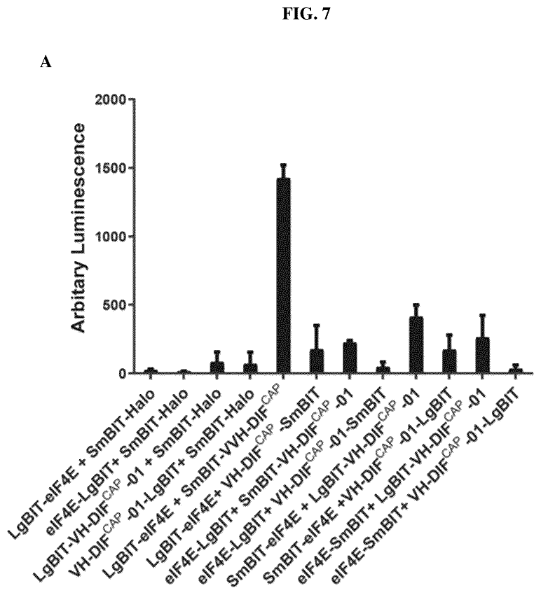

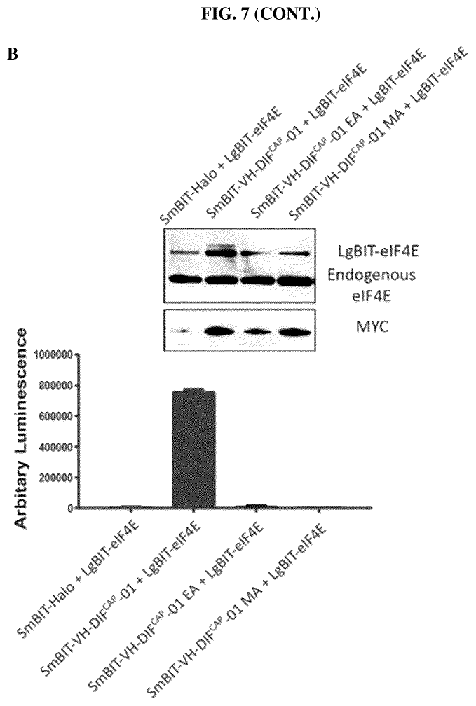

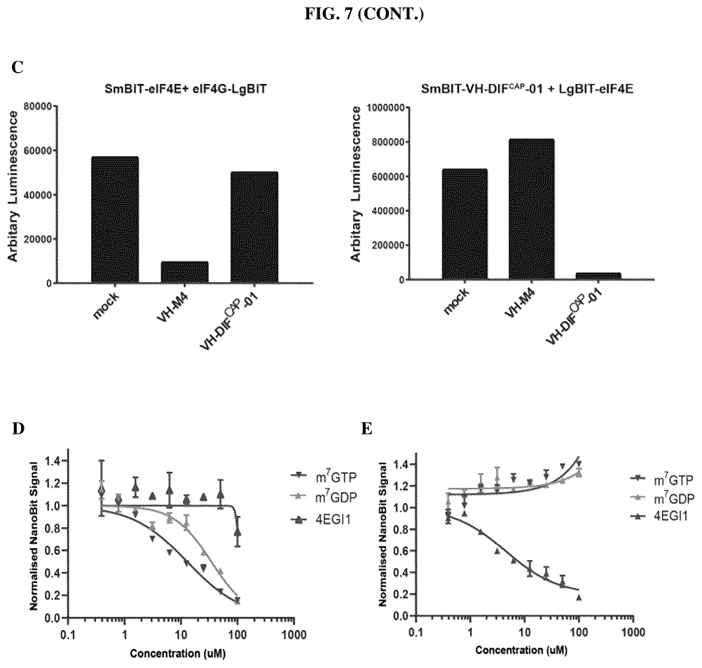

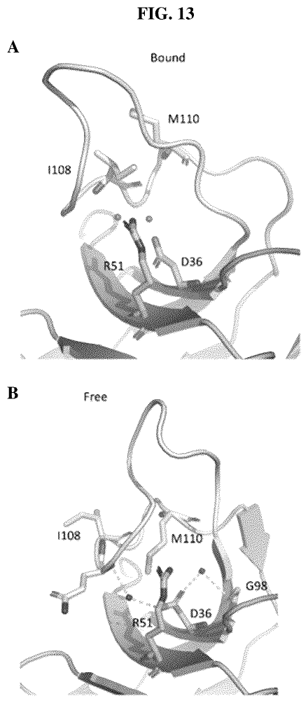

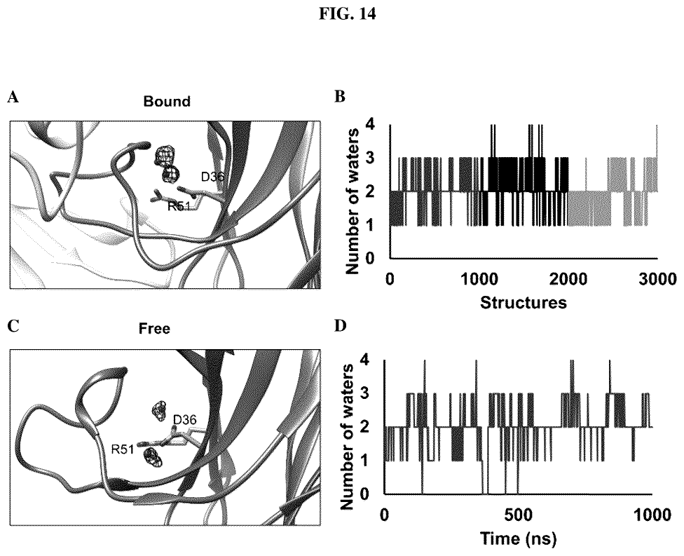

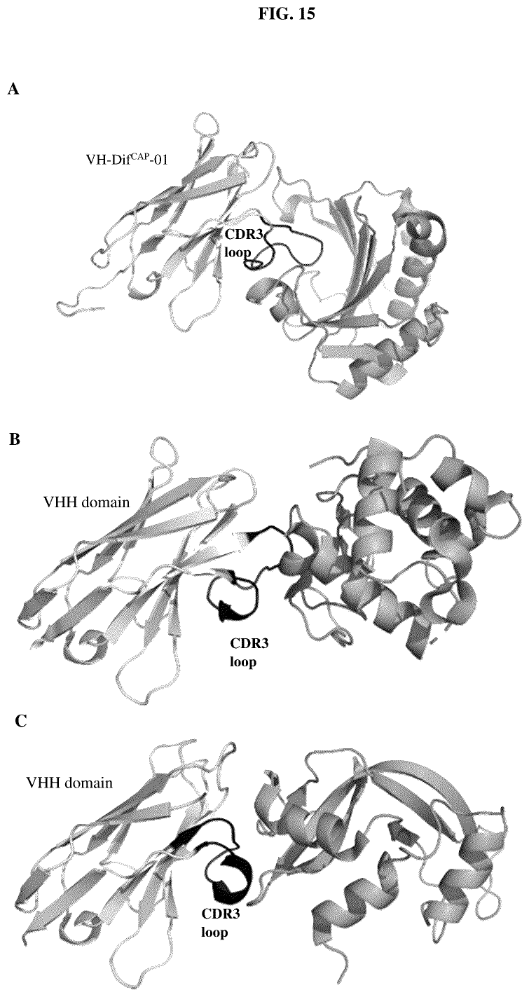

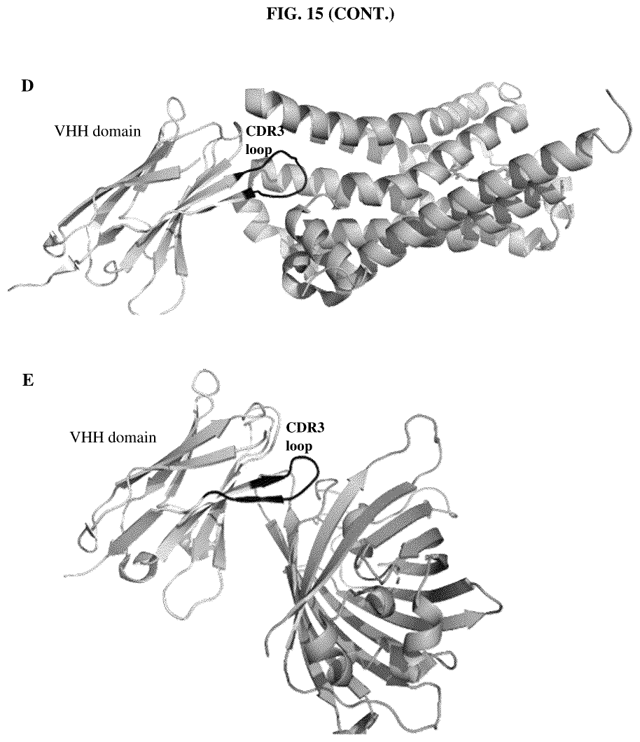

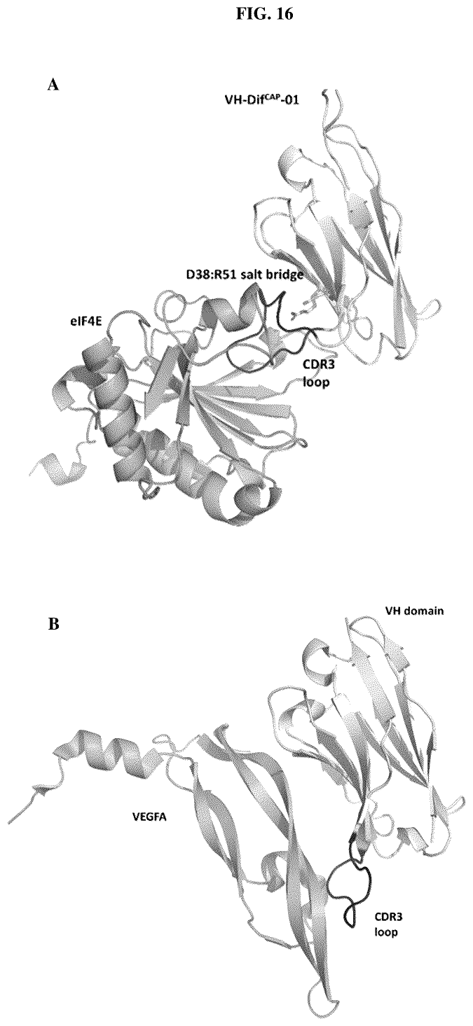

However, the most remarkable feature is the presence of two buried structure water molecules that allow the R51: D36 salt bridge to stabilize the fold of the CDR3 loop, when bound to eIF4E (FIG. 5J). These waters enable R51 and D36 to indirectly stabilize the polypeptide backbone of residue Ile110 and Q109 (FIG. 5J). Additionally, R51 also forms direct interactions with the carbonyl of F107 and the hydroxyl of the T108 sidechain, further rigidifying the presentation of the cap-site interaction motif (FIG. 5J). MD simulations also demonstrated that the unbound CDR3 loop of the VH-DiF^{CAP-01} domain undergoes a distinct structural change in comparison to the bound form (FIGS. 9C and 9D). The CDR3 loop undergoes a structural relaxation, whereby the β-hairpin structure associated with the ‘EMGFF’ motif is lost and instead there is a general movement of the CDR3 loop away from the body of the VH domain scaffold (FIG. 7). Interestingly, this movement is underpinned by significant structural changes in the hydrophobic core of the CDR3 loop structure with the L110 sidechain rotating out and the M113 sidechain rotating in to replace it. In association with these changes, the two buried structured waters observed in the bound form also adopt new position within the CDR3 loop, which help to stabilize the new conformation by forming 2 water mediated interactions between the amide backbones of Q109 and G98 with the D36 sidechain, respectively (FIG. 13). In both simulations of the bound and unbound forms of VH-DiF^{CAP-01} (FIG. 14), the two water positions remained predominately solvated indicating that water molecules found here exchanged slowly with the external solvent and formed enthalpically favorable interactions.

VH-DIF^{CAP-01} Inhibits eiF4F Mediated Cap Dependent Translation by Disrupting the Interplay Between eIF4E and Capped-mRNA

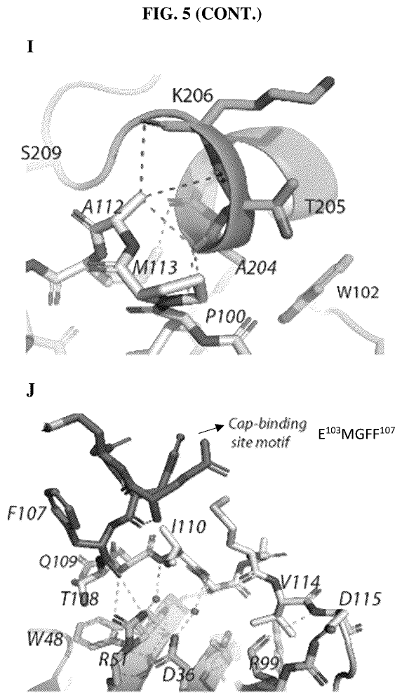

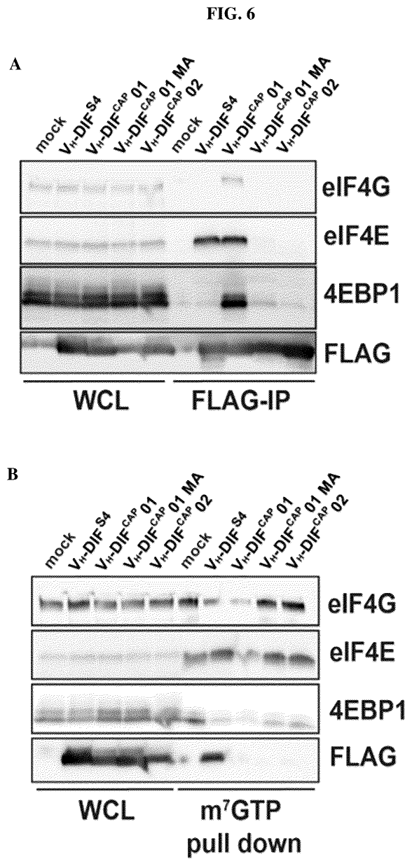

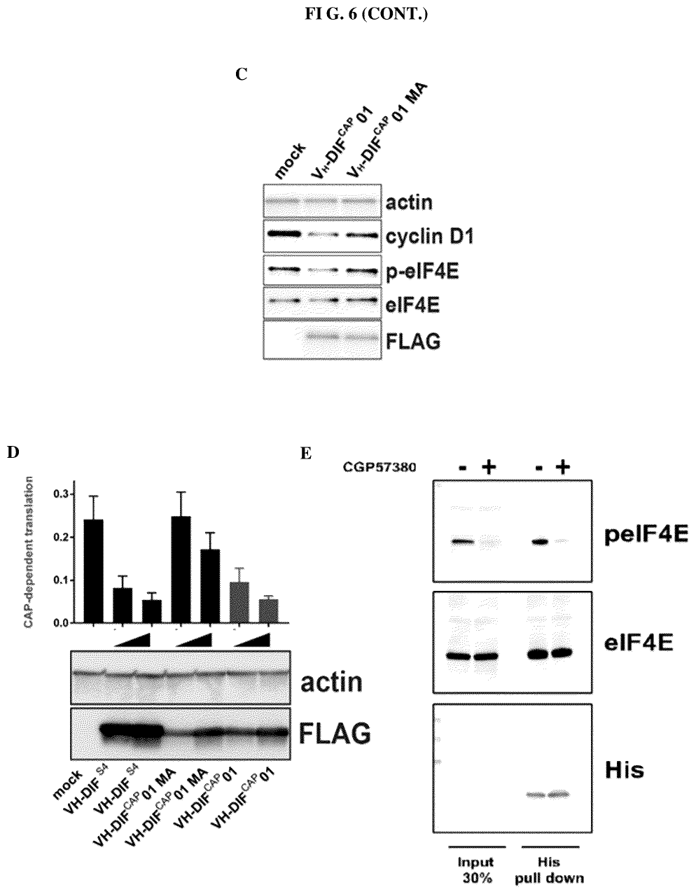

As predicted from in vitro studies, FLAG-tagged VH-DiF^{CAP-01} immuno-precipitated cellular eIF4E more efficiently than VH-DIF^{CAP-02} (FIG. 6A), a peptide aptamer that demonstrate little binding with eIF4E in vitro (table 3). Consistent with the ability of VH-DIF^{CAP-01} to interact with eIF4E at the cap-binding site, eIF4E also co-immunoprecipitated with endogenous eIF4G and 4EBP1 (FIG. 6A) This result correlated with the reduction of eIF4F complex formation on m⁷GTP beads when VH-DIF^{CAP-01} is over-expressed (FIG. 6B). Concomitantly with VH-DIF^{CAP-01} expression, a reduction in the levels of eIF4E phosphorylation at S209 was observed (FIG. 6C). This decrease in eIF4E phosphorylation implies that the VH-DIF^{CAP-01} interaction with eIF4E interfered with the eIF4G mediated recruitment of the MNK1/2 kinase to the eIF4F complex and in turn targeting of the S209 residue. 31 Mutation of the methionine residue (M104) to alanine, critical for the interaction of the cyclic peptide motif with eIF4E, abrogated the ability of VH-DIF^{CAP-01} to immuno-precipitate eIF4E and confirmed its specific effects on eIF4E phosphorylation. These results infer that either displacement of mRNA from the cap-binding site or steric occlusion caused by VH-DIF^{CAP-01} binding prevents MNK1/2 mediated phosphorylation of eIF4E. The effects of VH-DIF^{CAP-01} upon mRNA translation were assessed using a bi-cistronic luciferase reporter32, which demonstrated that the peptide aptamer specifically inhibited cap-dependent translation versus cap-independent translation (FIG. 6D). Additionally, cellular expression of VH-DIF^{CAP-01} also down-regulated Cyclin D1 protein levels (FIG. 6C). A protein that is considered to a hallmark to eIF4F signalling inhibition in cells. In contrast, the VH-DIF^{CAP-01} mutant (M104A, “VH-DIF^{CAP-01} MA”) constructs exhibited negligible activity in the bicistronic assay or on cyclin D1 protein expression (FIGS. 6C and 6D). Purified VH-DIF^{CAP-01} was also able to efficiently interact with both phosphorylated and unphosphorylated forms of eIF4E (FIG. 6E) in pull downs from cell lysate. Examination of the crystal structure demonstrates that phosphorylation of 209 would not impede the eIF4E:VH-DIF^{CAP-01} interaction (FIG. 5I).

Development of a Novel Live Cell Protein-Protein Interaction (PPI) Assay to Measure Antagonism of the eIF4E Cap-Binding Site.

Currently, there is no live cell-based assay that can evaluate engagement of the cap-binding site by small molecules or other modalities. A site that has been the target of multiple studies to develop cell permeable small molecules for therapeutic development. Fortunately, there is a plethora of suitable technologies that can be used to develop an appropriate assay e.g., split luciferase, BRET and FRET (bio and fluorescence resonance excitation transfer), and cellular localization technologies. The VH-DIF^{CAP-01} peptide aptamer in combination with the NanoLuc-based protein complementation system (NanoBit, PCA, Promega) was exploited to develop a PPI assay that can assess antagonism of the m⁷GTP cap-binding site in eIF4E in cells. The NanoLuc complementation protein system consists of two components termed LgBiT (18-kDa protein fragment) and SmBiT (11-amino-acid peptide fragment), which have been optimised for minimal self-association and stability. When LgBiT and SmBiT are optimally fused to two interacting proteins, both the fused proteins will be brought into proximity to each other by the resulting interaction, resulting in the formation of the active luciferase.

The LgBiT-eIF4E and SmBiT-VH-DIF^{CAP-01} were identified as the transfection pair that reconstituted the highest luciferase signal without exhibiting background activity in the negative controls, thus confirming that the NanoBit reporter fragments were not spontaneously assembling under the experimental conditions (FIGS. 7A and 7B). To further confirm the specificity of this assay for binding at the cap site, the NanoBit assay was re-performed with both the VH-DIF^{CAP}-01M104A (termed VH-DIF^{CAP-01} “MA”; SEQ ID NO: 102) and VH-DIF^{CAP-01} E103A (termed VH-DIF^{CAP-01} “EA; SEQ ID NO: 103) binding controls fused to smBIT, which resulted in the abrogation of the luciferase signal above background (FIG. 7B). Additionally, the ability of the assay to measure and differentiate between interactors that bound at the either the cap-binding or eIF4G binding sites of eIF4E was demonstrated by co-transfection of the NanoBit assay (termed NanoBIT eIF4ECAP) with either untagged VH-M4 (SEQ ID NO: 100), a VH-domain designed to interact with eIF4E at the eIF4G binding site, or VH-DIF^{CAP-01} (FIG. 7C).

To validate that the NanoBIT eIF4ECAP system can measure small molecule mediated inhibition of the eIF4E cap-binding site, the system was also used to screen two known cap-analogue antagonists (m⁷GTP and m⁷GDP) and an established cell permeable inhibitor of the eIF4E:4G interface (4EGI1) as a negative control. Unfortunately, both cap-analogue molecules are cell impermeable. Therefore, to circumvent this issue, a sub-CMC (critical micelle concentration) treatment of digitonin was used to permeabilize and enable cellular entry of the cap-analogues into HEK293 cells transfected with the NanoBIT^{CAP} system. In the permeabilized cells, both m⁷GTP and M7GDP disrupted the interaction of LgBiT-eIF4E with SmBiT-VH-DIF^{CAP-01} with IC50s of 12.8 μM and 34.5 μM respectively, whilst 4EGI1 had a negligible effect on the NanoBIT signal, demonstrating that the assay system can measure cap-binding site antagonists (FIG. 7D). To highlight the specificity of the NanoBIT^{CAP} system further, it was also shown that only 4EIG1 and neither of the two cap-analogues were able to inhibit the luciferase signal in an alternative NanoBIT system (eIF4E:eIF4G604-646) that measures disruption of the eIF4E:4G interface (FIG. 7E). Both sets of described experiments were then repeated in non-permeabilized cell, where as expected the impermeable cap-analogues elicited no effects, and the cell permeable only 4EGI1 inhibited the NanoBIT signal in the NanoBit eIF4E:eIF4G604-646 system.

the Peptide Aptamers 4AM3, 4AM14 and 4AM20 Interact with eIF4a.

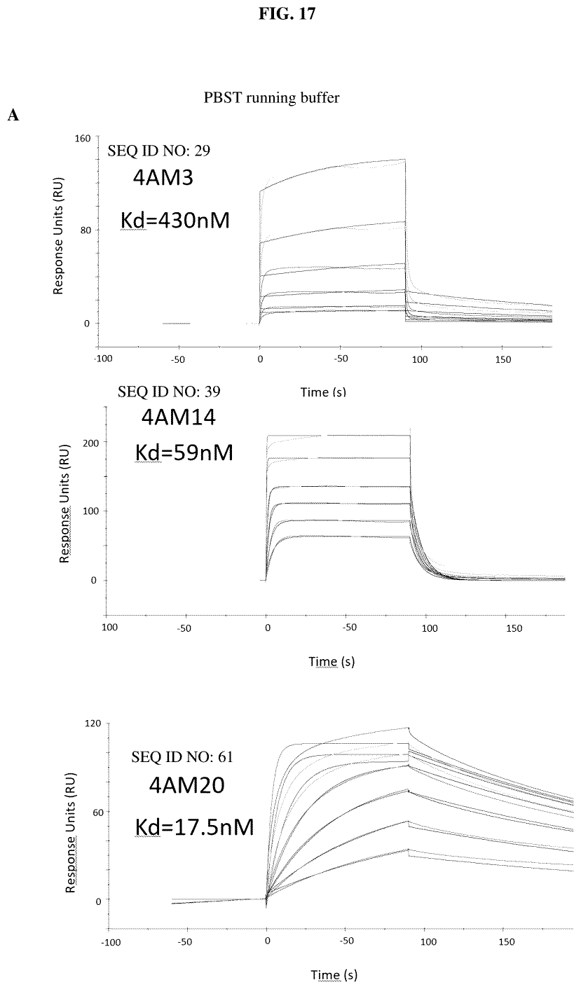

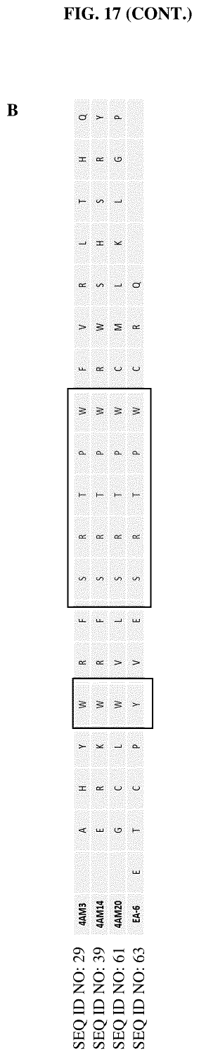

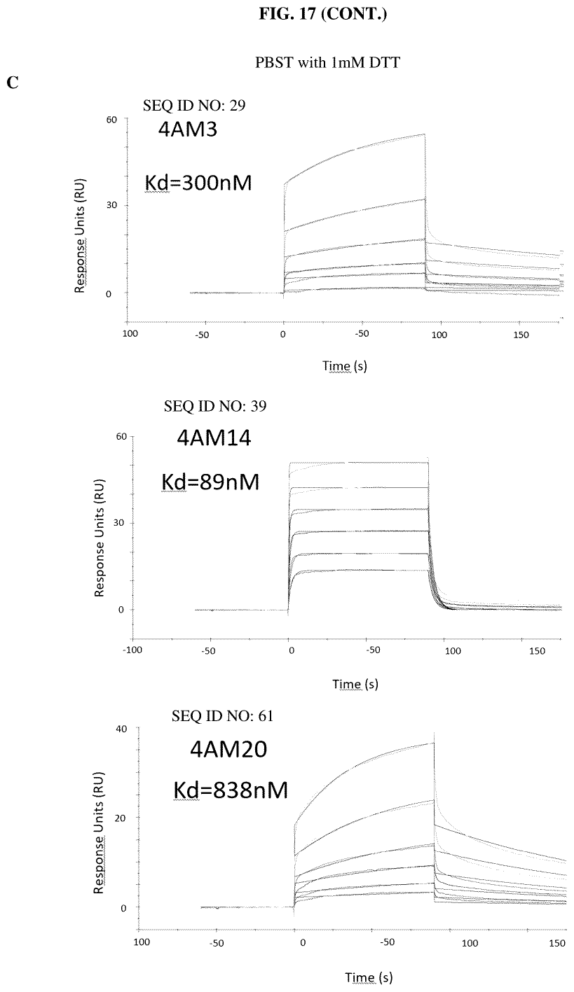

The interaction between the peptide aptamers (4AM3 (SEQ ID NO: 29), 4AM14 (SEQ ID NO: 39) and 4AM20 (SEQ ID NO: 61)) was measured by Surface Plasmon Resonance in PBST running buffer and PBST with 1 mM DTT. Amongst the three peptide aptamers, 4AM3 exhibited the highest K_{d} of 430 nM in PBS running buffer only (FIG. 17A) and 4AM20 displayed the highest K_{d} of 838 nM in PBST running buffer with 1 nM DTT (FIG. 17B) (see Table 4).

TABLE 4
Dissociation constants were determined by Surface Plasmon
Resonance in PBST running buffer with and without 1 mM DTT.

Dissociation Constant (K_{d})

| | PBST Running | | PBST with | |
|---|---|---|---|---|
| Solutions | Buffer | | 1 mM DTT | |
| 4AM3 (SEQ ID NO: 29) | 430 | nM | 300 | nM |
| 4AM14 (SEQ ID NO: 39) | 59 | nM | 89 | nM |
| 4AM20 (SEQ ID NO: 61) | 17.5 | nM | 838 | nM |

the Peptide Aptamers 4AM14 and 4AM20 Bind to eIF4a.

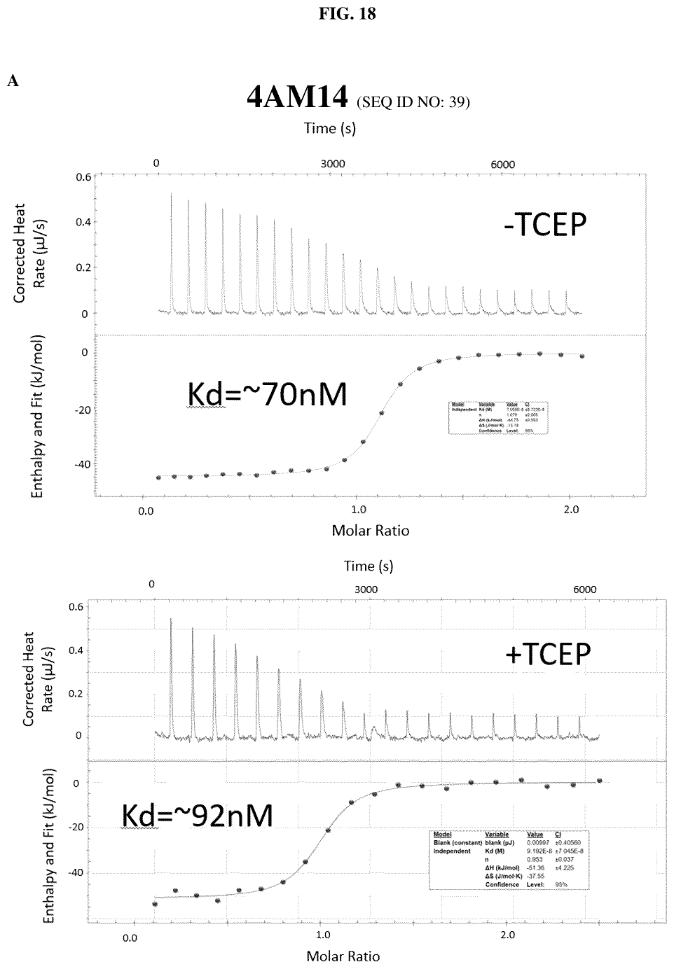

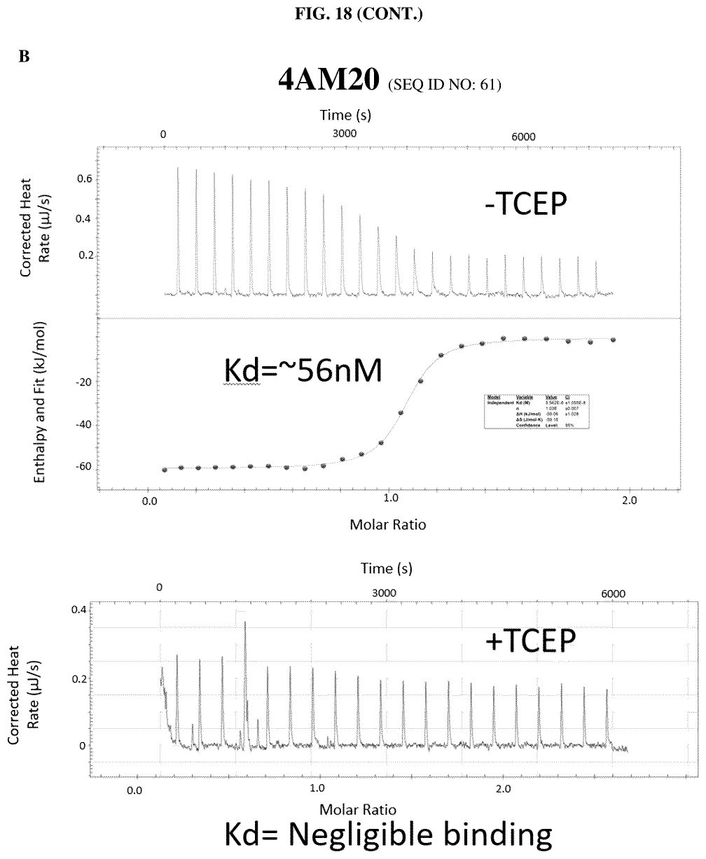

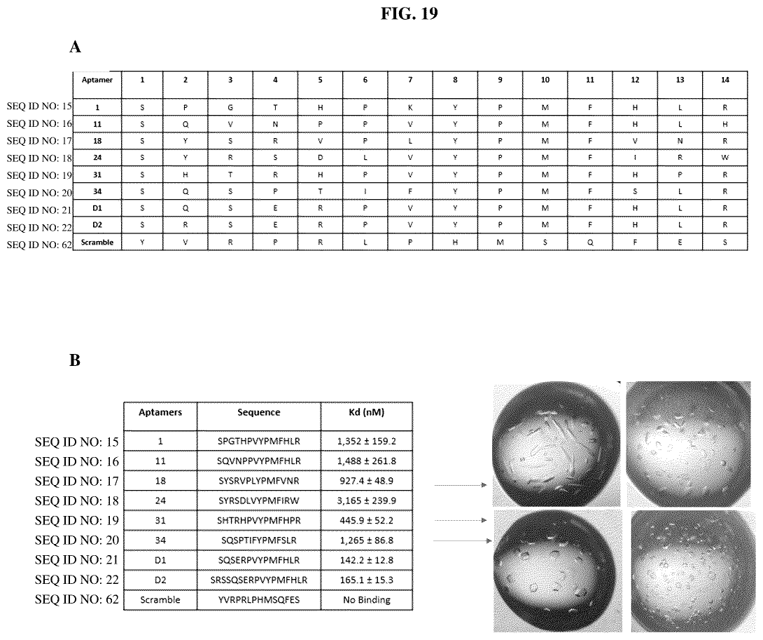

The binding of the peptide aptamers 4AM14 and 4AM20 to eIF4A is measured by Isothermal Titration calorimetry (ITC). 4AM14 displayed the K_{d} of approximately 70 nM without TCEP and approximately 92 nM in the presence of TCEP (FIG. 18A). For 4AM20, the binding to eIF4A without TCEP is approximately 56 nM and in the presence of TCEP is negligible (FIG. 18B).

### the Peptide Aptamers VH-D1 and VH-31 Inhibit Cell Proliferation by Binding to PCNA.

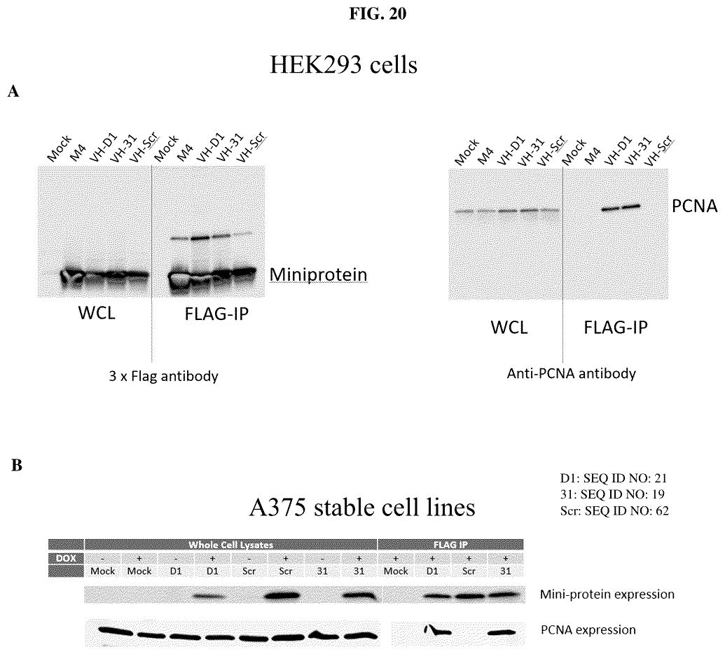

To investigate whether the peptide aptamers VH-D1 and VH-31 interact with PCNA, an anti-FLAG immunoprecipitation pull down assay was performed. FIG. 20A demonstrates that the specific interaction of FLAG tagged PCNA interacting peptide aptamers (VH-D1 and VH-31) can be pulled down by anti-FLAG conjugated beads in complex with PCNA, whilst the negative control M4 and scrambled do not. FIG. 20B shows that peptide aptamer expression in stably transfected A375 cells can be inducibly controlled with doxycycline. Additionally, the peptide aptamers (VH-D1 and VH-31) can be pulled down with PCNA in anti-FLAG IP experiments.

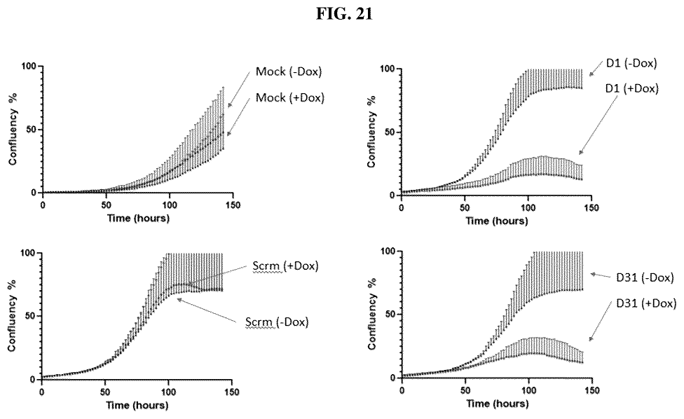

Furthermore, the effect of these peptide aptamers on the confluency of the cells was investigated. Induction of PCNA interacting aptamers (VH-D1 and VH-31) but not their controls reduces the proliferation of human melanoma cancer cells (A375 cells) (FIG. 21).

### Conclusion

The present invention shows the development of new methods to engineer and identify peptide aptamers without disulphide bonds that display improved cell permeability and stability whilst retaining high binding affinity to the target protein and peptide aptamers with disulphide bonds which are useful in diagnostic and purification applications. These peptide aptamers are compatible with RNA and DNA delivery technology. These results demonstrate that the PELE process can enable the development of mini-proteins that interact at desired target interaction sites, that can model and assess the potential effects of drug inhibition and allow the construction of critical target engagement assays that can accelerate the identification of lead compounds for drug development.

TABLE 4
Summary of sequence listing.

| Description | Polypeptide sequence |
|---|---|
| Amino acid sequence template 1 of peptide | XXXXXEMGFFXXXXX |
| aptamer (eIF4AE) (SEQ ID NO: 1) | |
| Amino acid sequence template 2 of peptide | XXXEMGFFXXXXXXX |
| aptamer (eIF4AE) (SEQ ID NO: 2) | |
| Amino acid sequence template 3 of peptide | XXXXXXXEMGFFXXX |
| aptamer (eIF4AE) (SEQ ID NO: 3) | |
| Amino acid sequence of peptide aptamer 1 | PLPEMGFFTNIPAMV |
| (eIF4AE) (VH-DiF^{CAP-01}) (SEQ ID NO: 4) | |
| Amino acid sequence of peptide aptamer 2 | PLYAPEMGFFHVHHL |
| (eIF4AE) (VH-DiF^{CAP-02}) (SEQ ID NO: 5) | |
| Amino acid sequence of peptide aptamer 3 | VNYLPEMGFFHTHSL |
| (eIF4AE) (VH-DiF^{CAP-03}) (SEQ ID NO: 6) | |
| Amino acid sequence of peptide aptamer 4 | FASEMGFFLVMTMGF |
| (eIF4AE) (VH-DiF^{CAP-04}) (SEQ ID NO: 7) | |
| Amino acid sequence of peptide aptamer 5 | MHPSAICEMGFFQDC |
| (eIF4AE) (VH-DiF^{CAP-05}) (SEQ ID NO: 8) | |
| Amino acid sequence of peptide aptamer 6 | PGSEMGFFSCRSLPL |
| (eIF4AE) (VH-DiF^{CAP-06}) (SEQ ID NO: 9) | |
| Amino acid sequence of peptide aptamer 7 | RLLPPEMGFFKPYWW |
| (eIF4AE) (VH-DiF^{CAP-07}) (SEQ ID NO: 10) | |
| Amino acid sequence of peptide aptamer 8 | PGSEMGFFACLWFYS |
| (eIF4AE) (VH-DiF^{CAP-08}) (SEQ ID NO: 11) | |
| Amino acid sequence of peptide aptamer 9 | MVPEMGFFEPGLPSP |
| (eIF4AE) (VH-DiF^{CAP-09}) (SEQ ID NO: 12) | |
| Amino acid sequence of peptide aptamer 10 | SEPEMGFFVLCAILQ |
| (eIF4AE) (VH-DiF^{CAP-10}) (SEQ ID NO: 13) | |
| Amino acid sequence template of peptide | XXXXXXXYPMFXXX |
| aptamer (PCNA) (SEQ ID NO: 14) | |
| Amino acid sequence of peptide aptamer 1 | SPGTHPKYPMFHLR |
| (PCNA) (VH-1)(SEQ ID NO: 15) | |
| Amino acid sequence of peptide aptamer 2 | SQVNPPVYPMFHLH |
| (PCNA) (VH-11) (SEQ ID NO: 16) | |
| Amino acid sequence of peptide aptamer 3 | SYSRVPLYPMFVNR |
| (PCNA) (VH-18) (SEQ ID NO: 17) | |
| Amino acid sequence of peptide aptamer 4 | SYRSDLVYPMFIRW |
| (PCNA) (VH-24) (SEQ ID NO: 18) | |
| Amino acid sequence of peptide aptamer 5 | SHTRHPVYPMFHPR |
| (PCNA) (VH-31) (SEQ ID NO: 19) | |
| Amino acid sequence of peptide aptamer 6 | SQSPTIFYPMFSLR |
| (PCNA) (VH-34) (SEQ ID NO: 20) | |
| Amino acid sequence of peptide aptamer 7 | SQSERPVYPMFHLR |
| (PCNA) (VH-D1) (SEQ ID NO: 21) | |
| Amino acid sequence of peptide aptamer 8 | SRSERPVYPMRFHLR |
| (PCNA) (VH-D2) (SEQ ID NO: 22) | |
| Amino acid sequence of peptide aptamer 9 | MSEVQLVESGGGLVQP |
| (PCNA) (SEQ ID NO: 23) | GGSLRLSSAISGFSIS |
| | STSIDWVRQAPGKGLE |
| | WVARISPSSGSTSYAD |
| | SVKGRFTISADTSKNT |
| | VYLQMNSLRAEDTAVY |
| | YTGRSHTRHPVYPMFH |
| | PRDYRGQGTLVTVSSG |
| | AAEQKLIFEEDL |
| Amino acid template sequence of peptide | XXXXXWXXSRTPWXXXXX |
| aptamer 1 (eIF4A) (SEQ ID NO: 24) | |
| Amino acid template sequence of peptide | XXXXXXXWXXSRTPWXXX |
| aptamer 2 (eIF4A) (SEQ ID NO: 25) | |
| Amino acid template sequence of peptide | XXXWXXSRTPWXXXXXXX |
| aptamer 3 (eIF4A) (SEQ ID NO: 26) | |
| Amino acid sequence of peptide aptamer 1 | LTWTRWVRSRTPWNVMGF |
| (eIF4A) (SEQ ID NO: 27) | |
| Amino acid sequence of peptide aptamer 2 | LVQRLTRWRFSRTPWHFW |
| (eIF4A) (SEQ ID NO: 28) | |
| Amino acid sequence of peptide aptamer 3 | AHYWRFSRTPWFVRLTHQ |
| (eIF4A) (4AM3) (SEQ ID NO: 29) | |
| Amino acid sequence of peptide aptamer 4 | IQRWRLSRTPWSRSFHRI |
| (eIF4A) (SEQ ID NO: 30) | |
| Amino acid sequence of peptide aptamer 5 | SLRWINSRTPWWIVRRML |
| (eIF4A) (SEQ ID NO: 31) | |
| Amino acid sequence of peptide aptamer 6 | TERWHDSRTPWANLYRHF |
| (eIF4A) (SEQ ID NO: 32) | |
| Amino acid sequence of peptide aptamer 7 | SVKWLNSRTPWTRMMYTL |
| (eIF4A) (SEQ ID NO: 33) | |
| Amino acid sequence of peptide aptamer 8 | RSAGLWRYSRTPWYLLRT |
| (eIF4A) (SEQ ID NO: 34) | |
| Amino acid sequence of peptide aptamer 9 | QLRWSTSRTPWAKLAVKP |
| (eIF4A) (SEQ ID NO: 35) | |
| Amino acid sequence of peptide aptamer 10 | VSSVRWFRSRTPWKMLHH |
| (eIF4A) (SEQ ID NO: 36) | |
| Amino acid sequence of peptide aptamer 11 | NFRWVWSRTPWHMRADVR |
| (eIF4A) (SEQ ID NO: 37) | |
| Amino acid sequence of peptide aptamer 12 | CYKWASSRTPWARLWATT |
| (eIF4A) (SEQ ID NO: 38) | |
| Amino acid sequence of peptide aptamer 13 | ERKWRFSRTPWRWSHSRY |
| (eIF4A) (4AM14) (SEQ ID NO: 39) | |
| Amino acid sequence of peptide aptamer 14 | SHHWFGSRTPWSWSNTLR |
| (eIF4A) (SEQ ID NO: 40) | |
| Amino acid sequence of peptide aptamer 15 | STKWSESRTPWALLRLSP |
| (eIF4A) (SEQ ID NO: 41) | |
| Amino acid sequence of peptide aptamer 16 | YPWLRWRLSRTPWSLRWS |
| (eIF4A) (SEQ ID NO: 42) | |
| Amino acid sequence of peptide aptamer 17 | GRAYPWRWSRTPWYRTRL |
| (eIF4A) (SEQ ID NO: 43) | |
| Amino acid sequence of peptide aptamer 18 | NLRAPYRWLKSRTPWANM |
| (eIF4A) (SEQ ID NO: 44) | |
| Amino acid sequence of peptide aptamer 19 | AIGRWWGWSRTPWRFPLP |
| (eIF4A) (SEQ ID NO: 45) | |
| Amino acid sequence of peptide aptamer 20 | SSRWTNSRTPWTLISLSD |
| (eIF4A) (SEQ ID NO: 46) | |
| Amino acid sequence of peptide aptamer 21 | VISWRWSRTPWSLRAKNR |
| (eIF4A) (SEQ ID NO: 47) | |
| Amino acid sequence of peptide aptamer 22 | VISWRWSRTPWSLRAKNR |
| (eIF4A) (SEQ ID NO: 48) | |
| Amino acid sequence of peptide aptamer 23 | AARWTNSRTPWSTYRWLK |
| (eIF4A) (SEQ ID NO: 49) | |
| Amino acid sequence of peptide aptamer 24 | KYVWKNSRTPWRRLTHNT |
| (eIF4A) (SEQ ID NO: 50) | |
| Amino acid sequence of peptide aptamer 25 | TATERWRNSRTPWTLIKV |
| (eIF4A) (SEQ ID NO: 51) | |
| Amino acid sequence of peptide aptamer 26 | QLRWLKSRTPWARVMLVL |
| (eIF4A) (SEQ ID NO: 52) | |
| Amino acid sequence of peptide aptamer 27 | VARRRWLGSRTPWVGKYS |
| (eIF4A) (SEQ ID NO: 53) | |
| Amino acid sequence of peptide aptamer 28 | GLRWRLSRTPWAAHLARG |
| (eIF4A) (SEQ ID NO: 54) | |
| Amino acid sequence of peptide aptamer 29 | SLRWRASRTPWNKLFVIL |
| (eIF4A) (SEQ ID NO: 55) | |
| Amino acid sequence of peptide aptamer 30 | FLRWTLSRTPWTVRAKNP |
| (eIF4A) (SEQ ID NO: 56) | |
| Amino acid sequence of peptide aptamer 31 | QWRWTWSRTPWRRYRRLS |
| (eIF4A) (SEQ ID NO: 57) | |
| Amino acid sequence of peptide aptamer 32 | MWRWVFSRTPWRRIQRDT |
| (eIF4A) (SEQ ID NO: 58) | |
| Amino acid sequence of peptide aptamer 33 | ITRWVNSRTPWHALLLLA |
| (eIF4A) (SEQ ID NO: 59) | |
| Amino acid sequence of peptide aptamer 34 | NFRWLNSRTPWTRANP |
| (eIF4A) (SEQ ID NO: 60) | |
| Amino acid sequence of peptide aptamer 35 | GCLWVLSRTPWCMLKLGP |
| (eIF4A) (4AM20) (SEQ ID NO: 61) | |
| Amino acid scramble sequence of peptide | YVRPRLPHMSQFES |
| aptamer (PCNA) (SEQ ID NO: 62) | |
| Amino acid sequence of peptide aptamer 36 | ETCPYVESRTPWCRQ |
| (eIF4A) (EA-6) (SEQ ID NO: 63) | |
| Amino acid sequence of peptide motif 1 for | CETGFFTGC |
| eIF4AE (EE-01) (SEQ ID NO: 64) | |
| Amino acid sequence of peptide motif 2 for | CELGYYNDC |
| eIF4AE (EE-02) (SEQ ID NO: 65) | |
| Amino acid sequence of peptide motif 3 for | CELGYYNDC |
| eIF4AE (EE-03) (SEQ ID NO: 66) | |
| Amino acid sequence of peptide motif 4 for | CETGFFLKC |
| eIF4AE (EE-04) (SEQ ID NO: 67) | |
| Amino acid sequence of peptide motif 5 for | CELGFYRLC |
| eIF4AE (EE-05) (SEQ ID NO: 68) | |
| Amino acid sequence of peptide motif 6 for | CETGFFLRC |
| eIF4AE (EE-06) (SEQ ID NO: 69) | |
| Amino acid sequence of peptide motif 7 for | CETGYFSQC |
| eIF4AE (EE-07) (SEQ ID NO: 70) | |
| Amino acid sequence of peptide motif 8 for | CIHSPTSLC |
| eIF4AE (SEQ ID NO: 71) | |
| Amino acid sequence of peptide motif 9 for | CETGFYKTC |
| eIF4AE (EE-08) (SEQ ID NO: 72) | |
| Amino acid sequence of peptide motif 10 for | CEMGYFGNC |
| eIF4AE (EE-09) (SEQ ID NO: 73) | |
| Amino acid sequence of peptide motif 11 for | CAMGFFQDC |
| eIF4AE (EE-02 E3A) (SEQ ID NO: 74) | |
| Amino acid sequence of peptide motif 12 for | CEAGFFQDC |
| eIF4AE (EE-02 M4A) (SEQ ID NO: 75) | |
| Amino acid sequence of peptide motif 13 for | CEMAFFQDC |
| eIF4AE (EE-02 G5A) (SEQ ID NO: 76) | |
| Amino acid sequence of peptide motif 14 for | CEMGAFQDC |
| eIF4AE (EE-02 F6A) (SEQ ID NO: 77) | |
| Amino acid sequence of peptide motif 15 for | CEMGFAQDC |
| eIF4AE (EE-02 F7A) (SEQ ID NO: 78) | |
| Amino acid sequence of peptide motif 16 for | CEMGFFADC |
| eIF4AE (EE-02 Q8A) (SEQ ID NO: 79) | |
| Amino acid sequence of peptide motif 17 for | CEMGFFQAC |
| eIF4AE (EE-02 D9A) (SEQ ID NO: 80) | |
| Amino acid sequence of peptide motif 18 for | CEMGFFQDCG |
| eIF4AE (EE-02 N-Del) (SEQ ID NO: 81) | |
| Amino acid sequence of peptide motif 19 for | ACEMGFFQDC |
| eIF4AE (EE-02 C-Del) (SEQ ID NO: 82) | |
| Amino acid sequence of peptide motif 20 for | CEMGFFQDC |
| eIF4AE (EE-02 NC-Del) (SEQ ID NO: 83) | |
| Amino acid sequence of peptide aptamer for | PLPEAGFFTNIPAMV |
| eIF4AE (Control) (VH-DiF^{CAP-01}Cntrl) (SEQ ID | |
| NO: 84) | |
| Amino acid sequence of peptide 1 with peptide | SLTYTREFLRSL |
| motif for eIF4AE (SEQ ID NO: 85) | |
| Amino acid sequence of peptide 2 with peptide | AQSYTRMELLRL |
| motif for eIF4AE (SEQ ID NO: 86) | |
| Amino acid sequence of peptide 3 with peptide | HYSRTDLLAYRW |
| motif for eIF4AE (SEQ ID NO: 87) | |
| Amino acid sequence of peptide 4 with peptide | SGIQQRMIVTWP |
| motif for eIF4AE (SEQ ID NO: 88) | |
| Amino acid sequence of peptide 5 with peptide | KLWSIPTNFLLP |
| motif for eIF4AE (SEQ ID NO: 89) | |
| 6Amino acid sequence of peptide 6 with | VEGQNLEALVTD |
| peptide motif for eIF4AE (SEQ ID NO: 90) | |
| Amino acid sequence of peptide 7 with peptide | TISYDRSTLFLF |
| motif for eIF4AE (SEQ ID NO: 91) | |
| Amino acid sequence of peptide 8 with peptide | DSAKERLAVSGT |
| motif for eIF4AE (SEQ ID NO: 92) | |
| Amino acid sequence of peptide 9 with peptide | LPRMMSDGEAGD |
| motif for eIF4AE (SEQ ID NO: 93) | |
| Amino acid sequence of peptide 10 with | YLMQTNQWQIAS |
| peptide motif for eIF4AE (SEQ ID NO: 94) | |
| Amino acid sequence of rational design of | GGSEMGFFGGSGG |
| peptide aptamer 1 for eIF4AE (SEQ ID NO: 95) | |
| Amino acid sequence of rational design of | GGSGGEMGFFGGS |
| peptide aptamer 2 for eIF4AE (SEQ ID NO: 96) | |
| Amino acid sequence of rational design of | GGSGEMGFFGGSG |
| peptide aptamer 3 for eIF4AE (SEQ ID NO: 97) | |
| Amino acid sequence of VH domain of peptide | MSEVQLVESGGGLVQPGGS |
| | LRLSSAISGFSISSTSIDWVRQ |
| aptamer 1 (eIF4AE) (VH-DiF^{CAP-01}) (SEQ ID NO: | APGKGLEWVARISPSSGSTS |
| 98) | YADSVKGRFTISADTSKNTV |
| | YLQMNSLRAEDTAVYYTGR |
| | PLPEMGFFTNIPAMVDYRGQ |
| | GTLVTVSSGAA |
| Amino acid sequence of VH domain (positive | MSEVQLVESGGGLVQPGGSLRL |
| control) (eIF4AE) (VH-1C5) (SEQ ID NO: 99) | SSAISGFSISSTSIDWVRQAPGK |
| | GLEWVARISPSSGSTSYADSVK |
| | GRFTISADTSKNTVYLQMNSLR |
| | AEDTAVYYTGRVAKADLNSSSPS |
| | FVVNTYSSFGFDYRGQGTLVTVS |
| | SGAA |
| Amino acid sequence of VH domain that binds | MSEVQLVESGGGLVQPGGSLRLS |
| at the eIF4AG interaction site of eIF4AE (VH-M4) | SAISGFSISSTSIDWVRQAPGKGLE |
| (SEQ ID NO: 100) | WVARISPSSGSTSYADSVKGRFTIS |
| | ADTSKNTVYLQMNSLRAEDTAVY |
| | YTGRVAKALNSSSPSFVVNTYSSF |
| | GFDYRGQGTLVTVSSGAA |
| Amino acid sequence of VH domain that | MSEVQLVESGGGLVQPGGSLRLS |
| interacts with eIF4AE at the eIF4AG binding | SAISGFSISSTSIDWVRQAPGKGLE |
| interface (eIF4AE) (VH-S4) (SEQ ID NO: 101) | WVARISPSSGSTSYADSVKGRFTIS |
| | ADTSKNTVYLQMNSLRAEDTAVY |
| | YTGRVAKALNSRSPSFVVNTYSSIG |
| | FDYRGQGTLVTVSSGAA |
| Amino acid sequence of VH domain of peptide | MSEVQLVESGGGLVQPGGSLRLSS |
| aptamer 1 (eIF4AE) with point mutation of | AISGFSISSTSIDWVRQAPGKGLEW |
| M104A (VH-DIF^{CAP-01} MA) (SEQ ID NO: 102) | VARISPSSGSTSYADSVKGRFTISAD |
| | TSKNTVYLQMNSLRAEDTAVYYTG |
| | RPLPEAGFFTNIPAMVDYRGQGTLV |
| | TVSSGAA |
| Amino acid sequence of VH domain of peptide | MSEVQLVESGGGLVQPGGSLRLSSA |
| aptamer 1 (eIF4AE) with point mutation of | ISGFSISSTSIDWVRQAPGKGLEWV |
| E103A (VH-DIF^{CAP-01} EA) (SEQ ID NO: 103) | ARISPSSGSTSYADSVKGRFTISADT |
| | SKNTVYLQMNSLRAEDTAVYYTGR |
| | PLPAMGFFTNIPAMVDYRGQGTLV |
| | TVSSGAA |
| Amino acid sequence of scrambled VH | MSEVQLVESGGGLVQPGGSLRLSSA |
| domain (negative control) (VH-1C5^{Scrambled}) | ISGFSISSTSIDWVRQAPGKGLEWVA |
| (SEQ ID NO: 104) | RISPSSGSTSYADSVKGRFTISADTS |
| | KNTVYLQMNSLRAEDTAVYYTGRGS |
| | VLSVAFKPSVDSFSSFYNTNDYRGQ |
| | GTLVTVSSGAA |

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1.-23. (canceled)

24. A peptide aptamer comprising an amino acid sequence selected from the group consisting of
i)
(SEQ ID NO: 24)
X₁X₂X₃X₄X₅WX₆X₇SRTPWX₈X₉X₁₀X₁₁X₁₂,
ii)
(SEQ ID NO: 25)
X₁₃X₁₄X₁₅X₁₆X₁₇X₁₈X₁₉WX₂₀X₂₁SRTPWX₂₂X₂₃X₂₄,
and
iii)
(SEQ ID NO: 26)
X₂₅X₂₆X₂₇WX₂₈X₂₉SRTPWX₃₀X₃₁X₃₂X₃₃X₃₄X₃₅X₃₆;
wherein X₁-X₃₆ is any amino acid;
wherein X₁₋₅, X₁₃₋₁₉ and X₂₅₋₂₉ comprise amino acid sequences for a first linker;
wherein X₈₋₁₂, X₂₂₋₂₄ and X₃₀₋₃₆ comprise amino acid sequences for a second linker;
optionally wherein X₁ and X₁₅ is an amino acid selected from the group consisting of leucine (L), glutamine (Q), arginine (R), valine (V), tyrosine (Y), glycine (G), alanine (A) and threonine (T);
optionally wherein X₂ and X₁₆ is an amino acid selected from the group consisting of threonine (T), arginine (R), serine (S), proline (P), alanine (A) and isoleucine (I);
optionally wherein X₃, X₁₇ and X₂₅ is an amino acid selected from the group consisting of tryptophan (W), leucine (L), alanine (A), isoleucine (I), serine (S), threonine (T), glutamine (Q), asparagine (N), glutamate (E), cysteine (C), proline (P), glycine (G), valine (V), arginine (K), arginine (R), phenylalanine (F), and methionine (M);
optionally wherein X₄, X₁₈ and X₂₆ is an amino acid selected from the group consisting of threonine (T), histidine (H), glutamine (Q), leucine (L), glutamate (E), valine (V), glycine (G), arginine (R), phenylalanine (F), tyrosine (Y), serine (S), isoleucine (I), alanine (A), tryptophan (W) and cysteine (C);
optionally wherein X₅, X₁₉ and X₂₇ is an amino acid selected from the group consisting of arginine (R), tyrosine (Y), lysine (K), leucine (L), histidine (H), proline (P), serine (S), tryptophan (W) and valine (V);
optionally wherein X₆, X₂₀ and X₂₈ is an amino acid selected from the group consisting of valine (V), arginine (R), isoleucine (I), histidine (H), leucine (L), serine (S), phenylalanine (F), alanine (A), glycine (G), threonine (T) and lysine (K);
optionally wherein X₇, X₂₁ and X₂₉ is an amino acid selected from the group consisting of asparagine (N), phenylalanine (F), tryptophan (W), leucine (L), alanine (A), glycine (G), lysine (K), glutamate (E), serine (S), arginine (R), threonine (T), tyrosine (Y) and aspartate (D);
optionally wherein X₈, X₂₂ and X₃₀ is an amino acid selected from the group consisting of asparagine (N), histidine (H), phenylalanine (F), serine (S), tryptophan (W), alanine (A), threonine (T), tyrosine (Y), lysine (K), arginine (R), valine (V) and cysteine (C);
optionally wherein X₉, X₂₃ and X₃₁ is an amino acid selected from the group consisting of valine (V), phenylalanine (F), arginine (R), isoleucine (I), asparagine (N), leucine (L), lysine (K), methionine (M), tryptophan (W), threonine (T), glycine (G) and alanine (A);
optionally wherein X₁₀, X₂₄ and X₃₂ is an amino acid selected from the group consisting of isoleucine (I), arginine (R), tyrosine (Y), leucine (L), valine (V), lysine (K), histidine (H), alanine (A), methionine (M), tryptophan (W), serine (S), threonine (T) and proline (P);
optionally wherein X₁₁ and X₃₃ is an amino acid selected from the group consisting of glycine (G), leucine (L), phenylalanine (F), arginine (R), tyrosine (Y), methionine (M), alanine (A), histidine (H), tryptophan (W), asparagine (N), lysine (K), serine (S), threonine (T) and glutamine (Q);
optionally wherein X₁₂ and X₃₄ is an amino acid selected from the group consisting of phenylalanine (F), threonine (T), histidine (H), arginine (R), tyrosine (Y), valine (V), aspartate (D), alanine (A), serine (S), leucine (L), proline (P), lysine (K) and tryptophan (W);
optionally wherein X₁₃ is an amino acid selected from the group consisting of asparagine (N) and leucine (L);
optionally wherein X₁₄ is an amino acid selected from the group consisting of valine (V), and leucine (L);
optionally wherein X₃₅ is an amino acid selected from the group consisting of (H), arginine (R), methionine (M), threonine (T), lysine (K), valine (V), leucine (L), serine (S), asparagine (N), isoleucine (I) aspartate (D) and glycine (G); and
optionally wherein X₃₆ is an amino acid selected from the group consisting of glutamine (Q), isoleucine (I), leucine (L), phenylalanine (F), proline (P), arginine (R), threonine (T), tyrosine (Y), aspartate (D), lysine (K), glycine (G), serine (S) and alanine (A);
wherein the peptide aptamer binds to eIF4A.

25. The peptide aptamer according to claim 24, wherein the peptide aptamer comprises the amino acid sequences selected from the group consisting of SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO:36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60 and SEQ ID NO: 61.

26. The peptide aptamer according to claim 24, wherein the peptide aptamer is not constrained by a disulphide bond.

27. The peptide aptamer according to claim 24, wherein the peptide aptamer is constrained by a disulphide bond.

28. The peptide aptamer according to claim 24, wherein the peptide aptamer comprises a peptide motif and/or one or more linker sequences located in a hypervariable region of a scaffold protein.

29. The peptide aptamer according to claim 28, wherein the scaffold protein is a VH domain.

30. (canceled)

31. A method of treating a condition associated with dysregulated cap-dependent translation, dysregulated DNA replication, dysregulated DNA repair and/or dysregulated mRNA translation, comprising administering the peptide aptamer according to claim 24 to a subject in need thereof, optionally comprising administering the peptide aptamer as a combinatorial treatment with immunotherapy.

32. The method according to claim 31, wherein the condition is selected from the group consisting of cancer, a disease associated with a viral infection and obesity.

33. The method according to claim 32, wherein the cancer is melanoma.

34.-40. (canceled)
